(19) United States
(12) Patent Application Publication
(10) Pub. No.: US 2025/0195552 A1
(43) Pub. Date: Jun. 19, 2025

(54) METHODS OF USING AVERMECTIN COMPOSITIONS FOR THE TREATMENT OF INFLAMMATORY DISORDERS AND DOSING REGIMENS

(71) Applicant: L4 Bio LLC, New York, NY (US)

(72) Inventors: Samuel D. Waksal, New York, NY (US); Rui Wu, Wilmington, DE (US); Alexandra Zanin-Zhorov, Staten Island, NY (US); Wei Chen, Beijing (CN); Julien Moretti, New York, NY (US)

(21) Appl. No.: 18/849,394

(22) PCT Filed: Mar. 22, 2023
(86) PCT No.: PCT/IB23/52816

§ 371 Date: Sep. 20, 2024

Related U.S. Application Data
(60) Provisional application No. 63/322,255, filed on Mar. 22, 2022.
(60) Provisional application No. 63/480,811, filed on Jan. 20, 2023.

(51) Int. Cl. A61K 31/7048; A61K 31/365; A61K 31/42; A61K 31/519; A61K 31/52; A61K 31/675; A61K 47/14; A61K 47/22

(52) U.S. Cl. CPC A61K 31/7048; A61K 31/365; A61K 31/42; A61K 31/519; A61K 31/52; A61K 31/675; A61K 47/14; A61K 47/22

(57) ABSTRACT

Disclosed are formulations and dosage forms of avermectins, and particularly of ivermectin. The disclosed compositions may be used in methods for the prevention, treatment, and control of various inflammatory disorders in humans.

### FIELD

The present disclosure provides compositions and dosage forms of avermectins, and particularly of ivermectin. The compositions may be used for the treatment and control of various inflammatory disorders in humans.

### BACKGROUND

The avermectins are a family of 16-membered macrocyclic lactone derivatives with potent anthelmintic and insecticidal properties and are used as active agents for the treatment or prevention of infection by parasitic worms and other parasitic infections. Avermectins are a series of macrolides, each of which is substituted thereon at the 13-position with a 4-(α-L-oleandrosyl)-α-L-oleandrose group. Avermectins are produced by cultures of the bacterium Streptomyces avermitilis or by synthetic or semi-synthetic means. The members of the avermectin family bind selectively and with high affinity to glutamate-gated chloride ion channels, which occur in invertebrate nerve and muscle cells. This leads to an increase in the permeability of the cell membrane to chloride ions with hyperpolarization of the nerve or muscle cell, resulting in paralysis and death of the parasite. All avermectin family of compounds show a similar spectrum of activity in different level of potency.

Ivermectin, an avermectin family member, is a highly potent anti-parasitic agent. Ivermectin is a mixture of 5-O-demethyl-22,23-dihydroavermectin Ala (also called 22,23-dihydroavermectin B1a) and 5-O-demethyl-25-de(1-methylpropyl)-22,23-dihydro-25-(1-methylethyl)avermectin A1a (also called 22,23-dihydroavermectin B1b). Ivermectin has been used historically as a broad-spectrum anti-parasitic medicinal product for human and veterinary use.

Ivermectin is commercially available for animal use as Cardomec (for felines), Eqvalane (for equines) and Ivomec (for bovines) by Merial; as Zimecterin (for equines) by Famam Companies, Inc. The medicine is available in tablets and chewables for heartworm prevention, topical solution for ear mite treatment, and injectable solution, oral paste or solution for other parasites in veterinary use. Ivermectin is also available for human use for treating parasitic infestations. For example, Stromectol, containing 3 mg ivermectin/tablet and marketed by Merck & Co., is approved by the U.S. Food and Drug Administration to treat onchoceriasis (river blindness) and strongyloidiasis (non-disseminated intestinal threadworm). Ivermectin may exert its antiparasitic activity via activation of a chloride ion-gated glutamate channel present in the invertebrate nervous system. Binding to the chloride ion-gated glutamate channel may result in hyperpolarization of nerves and muscle fiber. Such hyperpolarization may lead to paralysis and death of the organism (parasite). The chloride ion-gated glutamate channels are specific for invertebrates and are not expressed in the mammalian hosts, allowing for a specific action of ivermectin to be directed at the parasites.

The currently used medications for inflammatory disorders, including neuroinflammatory disorders such as Alzheimer's disease (AD), Parkinson's disease (PD), stroke, seizure disorders, epilepsy, traumatic brain injury, and amyotrophic lateral sclerosis (ALS), as well as autoimmune pathologies, such as limbic encephalitis, rheumatoid arthritis (RA), multiple sclerosis (MS), systemic lupus erythematosus (SLE), psoriasis, Crohn's disease and development of graft-versus-host disease (GVHID), show significant side effects such as behavioral changes, lethargy, insomnia, clinical depression, psychotic behavior, respiratory depression, coma and death, particularly when taken in overdoses or for long periods of time. Thus, there is an unmet need for development of alternative drugs that can be used to treat or prevent such inflammatory disorders that cause little or no adverse health risks.

### SUMMARY

The present disclosure provides pharmaceutical compositions and dosage forms of avermectins, and particularly of ivermectin. The present disclosure also provides methods for the treatment of various inflammatory disorders by administering to a patient in need thereof a composition comprising one or more avermectins, and particularly ivermectin. The compositions of the avermectins may be liquid or semi-solid at room temperature and comprise an avermectin, and particularly ivermectin, a first surfactant and a second surfactant. Also provided are dosing regimens for using the disclosed compositions and dosage forms for the treatment and prevention of inflammatory disorders. The methods, compositions and dosing regimens provided by this disclosure may address the need for avermectin formulations with improved pharmacokinetic properties, including, but not limited to bioavailability.

The disclosure provides a method of treating or preventing an inflammatory disorder, the method comprising administering to a subject in need thereof a pharmaceutical composition comprising:
(i) about 1% to about 15% of a compound of Formula I:

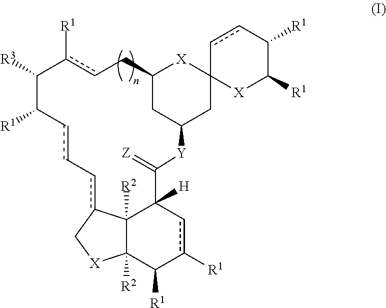

or a pharmaceutically acceptable salt, stereoisomer, polymorph, solvate, or isotopically labeled compound thereof, wherein
each occurrence of X is independently selected from —CH₂—, —NH—, —O—, —S—, —SO— and —SO₂—;
Y is selected from —CH₂—, —O—, —NH—, and —S—;
Z is selected from O and S;
each occurrence of [symbol] is a single bond or a double bond;
n is an integer 0-6; and [symbol]
each occurrence of R¹ is independently selected from halogen, —R, —OR, —NO₂, —NCS, —CN, —CF₃, —OCF₃, —NHR, —N(R)₂, —OC(O)R, —C(O)OR, —SR, —C(O)R, —C(O)C(O)R, —C(O)CH₂C(O)R, —C(S)R, —C(S)OR, —C(O)C(O)OR, —C(O)C(O)N(R)₂, —C(O)N(R)₂, —OC(O)N(R)₂, —C(S)N(R)₂, —N(R)C(O)OR, —N(R)C(O)R, —N(R)C(S)R, —N(R)C(O)N(R)₂, —N(R)C(S)N(R)₂, —N(COR)COR, —N(OR)R, —C(═NH)N(R)₂, —C(O)N(OR)R, —C(═NOR)R, —OP(O)(OR)₂, —P(O)(R)₂, —P(O)(OR)₂, —P(O)(H)(OR), —CH₂—OR, and —CH₂—O—CH₂—R;
each occurrence of R² is independently selected from independently selected from H, OH, O—C₁₋₄alkyl, —OC(O)C₁₋₄alkyl, —OC(O)NH₂, and —OC(O)NHC₁₋₄alkyl;
each occurrence of R³ is mono, di, or triglycoside, or OC(O)—(C₃-C₅)alkenyl;
each R is independently selected from H, —(C₁-C₁₂)alkyl, —(C₃-C₁₀)-cycloalkyl, (C₃-C₁₀)-cycloalkenyl, —[(C₃-C₁₀)cycloalkyl]-(C₁-C₁₂)alkyl, —[(C₃-C₁₀)cycloalkenyl]-(C₁-C₁₂)alkyl, —[(C₃-C₁₀)cycloalkenyl]-(C₁-C₁₂)alkyl, —[(C₃-C₁₀)cycloalkyl]-O—(C₁-C₁₂)alkyl, —[(C₃-C₁₀)cycloalkenyl]-O—(C₁-C₁₂)alkyl, —(C₆-C₁₀)aryl, (C₆-C₁₀)aryl-(C₁-C₁₂)alkyl, —(C₆-C₁₀)aryl-O—(C₁-C₁₂)alkyl, (C₆-C₁₀)aryl-N(R″)—(C₁-C₁₂)alkyl, 3- to 10-membered heterocyclyl, (3- to 10-membered heterocyclyl)-(C₁-C₁₂)alkyl, (3- to 10-membered heterocyclyl)-O—(C₁-C₁₂)alkyl, (3- to 10-membered heterocyclyl)-N(R″)—(C₁-C₁₂)alkyl, 5- to 10-membered heteroaryl, (5- to 10-membered heteroaryl)-(C₁-C₁₂)-alkyl, (5- to 10-membered heteroaryl)-O—(C₁-C₁₂)-alkyl and (5- to 10-membered heteroaryl)-N(R″)—(C₁-C₁₂)-alkyl;
   each heterocyclyl has 1-4 heteroatoms independently selected from N, NH, O, S, SO, and SO₂, and
   heteroaryl has 1-4 heteroatoms independently selected from N, NH, O, and S;
each occurrence of R is independently unsubstituted or is substituted with 1 to 5 R′; each occurrence of R′ is halo, OH, oxo, —CH₂OR″, —CH₂N(R″)₂, —C(O)N(R″)₂, —C(O)OR″, —NO₂, —NCS, —CN, —CF₃, —OCF₃ and —N(R″)₂; and each occurrence of R″ is independently H, C₁₋₆alkyl, C₂₋₆alkenyl, C₃₋₆cycloalkyl, C₃₋₆cycloalkenyl, 3- to 6-membered heterocyclyl, 5- to 10-membered heteroaryl, and (C₆-C₁₀)-aryl;
(ii) about 20% to about 40% of a first surfactant comprising one or more of:
   (a) mono-, di-, and/or tri-fatty acid esters of glycerol;
   (b) mono- and/or di-fatty acid esters of 1,2-propylene glycol; and
   (c) mono- and/or di-fatty acid esters of polyethylene glycol wherein the fatty acids are selected from C₆ to C₁₀ fatty acids; and
(iii) about 15% to about 70% of a second surfactant selected from one or more of a polysorbate surfactant and/or a fatty acid ester of sorbitan.

In some embodiments, the compound of Formula I is a compound of Formula II:

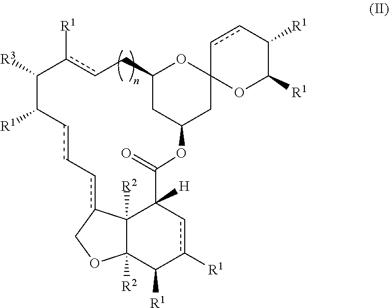

or a pharmaceutically acceptable salt, stereoisomer, polymorph, solvate or isotopically labeled compound thereof, wherein n, R¹, R², and R³ are each as defined in Formula I.

In some embodiments, the compound of Formula II is a compound of Formula III:

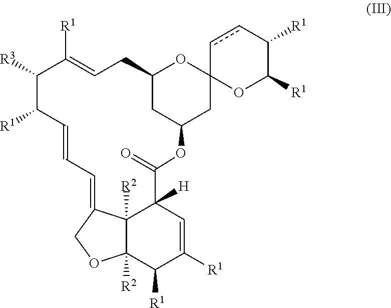

or a pharmaceutically acceptable salt, stereoisomer, polymorph, solvate or isotopically labeled compound thereof, wherein R¹, R², and R³ are each as defined in Formula I.

In some embodiments, the compound of Formula III is a compound of Formula IV:

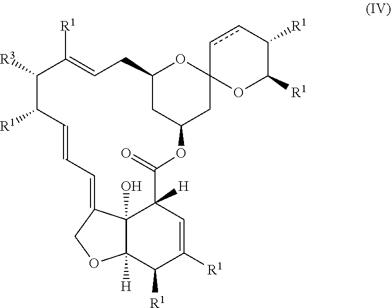

or a pharmaceutically acceptable salt, stereoisomer, polymorph, solvate or isotopically labeled compound thereof, wherein
each occurrence of R¹ is independently selected from halogen, —R, —OR, —NO₂, —NCS, —CN, —CF₃, —OCF₃, —NHR, —N(R)₂, —OC(O)R, —C(O)OR, —C(O)N(R)₂, —OC(O)N(R)₂, —N(R)C(O)OR, —N(R)C(O)R, —N(R)C(O)N(R)₂, —CH₂—OR, and —CH₂—O—CH₂—R;
each occurrence of R³ is mono, di, or triglycoside, or OC(O)—(C₃-C₅) alkenyl; and
R, R′ and R″ are each as defined in Formula I.

In some embodiments, the compound of Formula IV is a compound of Formula V:

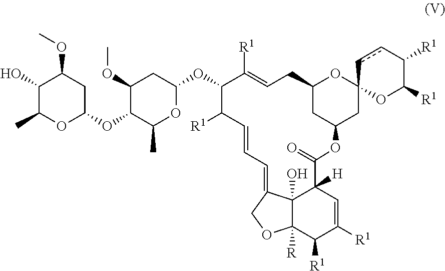

or a pharmaceutically acceptable salt, stereoisomer, polymorph, solvate or isotopically labeled compound thereof, wherein
each occurrence of R¹ is independently selected from halogen, —R, —OR, —NO₂, —NCS, —CN, —CF₃, —OCF₃, —NHR, —N(R)₂, —OC(O)R, —C(O)OR, —C(O)N(R)₂, —OC(O)N(R)₂, —N(R)C(O)OR, —N(R)C(O)R, —N(R)C(O)N(R)₂, —CH₂—OR, and —CH₂—O—CH₂—R; and
R, R′ and R″ are each as defined in Formula I.

In embodiments, the pharmaceutical composition comprises about 1% to about 15% of a compound of any one of Formulas I-VII. In some embodiments, the pharmaceutical composition comprises about 1% to about 15% of one or more of a compound of Formulas I-VII. In some embodiments, the pharmaceutical composition comprises about 1% to about 15% of ivermectin comprising a compound of Formula VI (22,23-dihydroavermectin B1a) and a compound of Formula VII (22,23-dihydroavermectin B1b). In some embodiments, the pharmaceutical composition comprises ivermectin comprising at least about 70% of 22,23-dihydroavermectin B1a and less than about 30% of 22,23-dihydroavermectin Bib. In some embodiments, the pharmaceutical composition comprises ivermectin comprising at least about 90% of 22,23-dihydroavermectin B1a and less than about 10% of 22,23-dihydroavermectin B1b.

In some embodiments, the pharmaceutical composition comprises about 3% to about 12% of a compound of any one of Formulas I-VII. In some embodiments, the pharmaceutical composition comprises about 5% to about 10% of a compound of any one of Formulas I-VII. In some embodiments, the pharmaceutical composition comprises about 1%, about 2%, about 3%, about 4%, about 5%, about 6%, about 7%, about 8%, about 9%, about 10%, about 11%, or about 12% of a compound of any one of Formulas I-VII.

In some embodiments, the pharmaceutical composition comprises about 3% to about 12% of one or more of a compound of Formulas I-VII. In some embodiments, the pharmaceutical composition comprises about 5% to about 10% of one or more of a of Formulas I-VII. In some embodiments, the pharmaceutical composition comprises about 1%, about 2%, about 3%, about 4%, about 5%, about 6%, about 7%, about 8%, about 9%, about 10%, about 11%, or about 12% of one or more of a of Formulas I-VII.

In some embodiments, the pharmaceutical composition comprises about 3% to about 12% of ivermectin comprising a compound of Formula VI and a compound of Formula VII. In some embodiments, the pharmaceutical composition comprises about 5% to about 10% ivermectin comprising a compound of Formula VI and a compound of Formula VII. In some embodiments, the pharmaceutical composition comprises about 1%, about 2%, about 3%, about 4%, about 5%, about 6%, about 7%, about 8%, about 9%, about 10%, about 11%, or about 12% ivermectin comprising a compound of Formula VI and a compound of Formula VII.

In embodiments, the pharmaceutical composition includes a first surfactant, wherein the fatty acids are selected from C₈ to C₁₀ fatty acids. In some embodiments, the first surfactant comprises mono- and di-fatty acid esters of glycerol. In some embodiments, the first surfactant is selected from Masester M8120, Capryol 90, Labrasol ALF, and combinations thereof. In some embodiments, the pharmaceutical composition includes about 25% to about 30% of the first surfactant.

In embodiments, the pharmaceutical composition includes a second surfactant selected from polysorbate 80 (Tween 80), sorbitan monolaurate (Span 20), and combinations thereof. In some embodiments, the pharmaceutical composition includes about 15% to about 20% of the second surfactant. In some embodiments, the pharmaceutical composition includes about 30% to about 35% of the second surfactant. In some embodiments, the pharmaceutical composition includes about 60% to about 65% of the second surfactant.

In some embodiments, the pharmaceutical composition comprises about 5% to about 55% D-α-Tocopherol polyethylene glycol 1000 succinate (vitamin E TPGS); or about 30% to about 50% vitamin E TPGS. In an embodiment, the pharmaceutical composition is in a dosage form comprising a gelatin capsule.

In embodiments, the pharmaceutical composition comprises: (i) about 5% to about 10% ivermectin; (ii) about 25% to about 30% mono- and di- C₈ to C₁₀ fatty acid esters of glycerol; (iii) about 15% to about 30% of a polysorbate surfactant; and (iv) about 30% to about 50% vitamin E TPGS.

In an embodiment, the pharmaceutical composition comprises: (i) about 8% ivermectin; (ii) about 27.6% mono- and di- C₈ to C₁₀ fatty acid esters of glycerol; (iii) about 32.2% polysorbate 80; and (iv) about 32.2% vitamin E TPGS.

In an embodiment, the pharmaceutical composition comprises: (i) about 8% ivermectin; (ii) about 27.6% Masester E8120; (iii) about 32.2% polysorbate 80; and (iv) about 32.2% vitamin E TPGS.

In an embodiment, the pharmaceutical composition comprises: (i) about 8% ivermectin; (ii) about 27.6% mono- and di- C₈ to C₁₀ fatty acid esters of glycerol; (iii) about 18.4% polysorbate 80; and (iv) about 46% vitamin E TPGS.

In an embodiment, the pharmaceutical composition comprises: (i) about 8% ivermectin; (ii) about 27.6% Masester E8120; (iii) about 18.4% polysorbate 80; and (iv) about 46% vitamin E TPGS.

In an embodiment, the pharmaceutical composition comprises: (i) about 8% ivermectin; (ii) about 27.6% mono- and di- C₈ to C₁₀ fatty acid esters of glycerol; and (iii) about 64.4% polysorbate 80.

In an embodiment, the pharmaceutical composition comprises: (i) about 8% ivermectin; (ii) about 27.6% Masester E8120; and (iii) about 64.4% polysorbate 80.

The disclosure provides a method of treating or preventing an inflammatory disorder comprising administering to a subject in need thereof a pharmaceutical composition disclosed herein. In an embodiment, the inflammatory disorder is an autoimmune disorder. In some embodiments, the autoimmune disorder is limbic encephalitis, rheumatoid arthritis (RA), systemic lupus erythematosus (SLE), Psoriasis, Crohn's disease or graft-versus-host disease (GVHD). In an embodiment, the inflammatory disorder is a neuroinflammatory disorder. In some embodiments, the neuroinflammatory disorder is Alzheimer's disease (AD), Parkinson's disease (PD), stroke, seizure disorders, epilepsy, infantile spasms, or amyotrophic lateral sclerosis (ALS). In one embodiment, the neuroinflammatory disorder is epilepsy. In some embodiments, the subject has focal seizures. In some embodiments, the subject has generalized tonic-clonic seizures.

In an embodiment, the subject is a mammal, particularly a human.

Provided is a method of treating an inflammatory disorder, the method comprising administering about 10 mg to about 120 mg of a compound disclosed herein to the subject. In some embodiments, about 10 mg to about 80 mg of the compound is administered to the subject. In some embodiments, about 20 mg to about 40 mg of the compound is administered to the subject. In some embodiments, about 10 mg, about 20 mg, about 40 mg, about 60 mg, about 80 mg, or about 120 mg of the compound is administered to the subject. In some embodiments, about 20 mg of the compound is administered to the subject. In some embodiments, about 60 mg of the compound is administered to the subject. In some embodiments, the pharmaceutical composition comprising a compound disclosed herein is administered once a day, every other day, or every three days. In an embodiment, the pharmaceutical composition comprising a compound disclosed herein is administered once a day. In some embodiments, the pharmaceutical composition is administered for at least 14 days. In some embodiments, the pharmaceutical composition is administered for about 14 days, for about 30 days, for about 60 days, for about 84 days, for about 90 days, or continuously. In an embodiment, the pharmaceutical composition is administered as a single dose on each day the pharmaceutical composition is administered. In an embodiment, the pharmaceutical composition is administered in the form of several divided doses on each day the pharmaceutical composition is administered.

In an embodiment, the method further comprises administering to the subject another therapeutic agent.

Provided herein is a method of assessing the efficacy of treating an inflammatory disorder with a composition described herein, the method comprising:
(a) obtaining a sample from a patient that has received treatment with the composition described herein;
(b) measuring the level of one or more inflammatory cytokines in the sample; and
(c) if the level of the one or more inflammatory cytokines in the sample is lower than a control level for the one or more inflammatory cytokines, determining that the patient is responsive to the treatment.

In some embodiments, the one or more inflammatory cytokines are selected from IL-1β, IL-6, IL-10, IL-12p70, IL-17, IL-21, IL-23, IFN-α, IFN-γ, TNF-α, and CXCL13. In some embodiments, the control level for the one or more inflammatory cytokines is the level for the one or more inflammatory cytokines obtained from the same patient before the start of treatment. In an embodiment, the inflammatory disorder is an autoimmune disorder. In some embodiments, the autoimmune disorder is limbic encephalitis, rheumatoid arthritis (RA), systemic lupus erythematosus (SLE), Psoriasis, Crohn's disease or graft-versus-host disease (GVHD). In an embodiment, the inflammatory disorder is a neuroinflammatory disorder. In some embodiments, the neuroinflammatory disorder is Alzheimer's disease (AD), Parkinson's disease (PD), stroke, seizure disorders, epilepsy, infantile spasms, or amyotrophic lateral sclerosis (ALS). In one embodiment, the neuroinflammatory disorder is epilepsy. In some embodiments, the subject has focal seizures. In some embodiments, the subject has generalized tonic-clonic seizures.

### BRIEF DESCRIPTION OF THE FIGURES

FIG. 1. Graph showing fiberoptic dispersion of ivermectin formulation prototypes in FaSSIF (1E-7E).

FIG. 2. The mean concentration-time curve of Ivermectin B1a in SD rat plasma after a single fasting intravenous injection with 2 mg/kg Ivermectin API.

FIG. 3. The mean concentration-time curve of Ivermectin B1a in SD rat plasma after a single fasting oral administration with 2 mg/kg Stromectol.

FIG. 4. The mean concentration-time curve of Ivermectin B1a in SD rat plasma after a single fasting oral administration with 2 mg/kg Ivomec.

FIG. 5. The mean concentration-time curve of Ivermectin B1a in SD rat plasma after a single fasting oral administration with 2 mg/kg formulation 7E.

FIGS. 6A and 6B. illustrate inhibition by ivermectin of IL-17, IL-21, IFN-γ, and CXCL13 secretion in human Th17-skewed CD4⁺ T cells. Human CD4⁺ T cells were stimulated with antibodies against CD3 and CD28 (anti-CD3/CD28) in combination with IL-1β and TGF-β (Th17-skewing conditions). FIG. 6A. Ivermectin inhibited IL-17, IL-21, and IFN-γ secretion Th17-skewed human CD4⁺ T cells in a dose-dependent manner with EC50s of 450 nM, 364 nM and 541 nM, respectively. FIG. 6B. Additional experiment showing results for CXCL13 (EC of 378 nm).

FIG. 7. Reduction of Th17-induced STAT3 and IkBα phosphorylation by ivermectin. Peripheral blood CD4⁺ T cells were treated with indicated doses of ivermectin and then stimulated by anti-CD3/28 mAbs for 2 hours. The cell lysates were prepared and analyzed by Western Blot. One representative of three different experiments is shown.

FIGS. 8A and 8B. Upregulation of FoxP3 mRNA in Th17-skewed peripheral blood CD4⁺ T cells and increase in the percentage of FoxP3⁺ T cells by ivermectin. CD4⁺ T cells were either stimulated by anti-CD3/28 mAbs, IL-1β and TGF-β for 5 days (Th17 act) or first treated with 1.5 mM of ivermectin and then stimulated by anti-CD3/28 mAbs, IL-1β and TGF-β for 5 days (Th17 act+ivermectin). FIG. 8A. FoxP3, IL-17A and RORc mRNA expression levels were determined by RT-PCR. FIG. 8B. The percentage of FoxP3⁺ T cells was determined by staining the FoxP3⁺ T cells using a PE-conjugated anti-FoxP3 antibody and counting the PE positive cells by Flow Cytometry.

FIG. 9. Inhibition by ivermectin of IL-17 and IFN-γ secretion in human PBMCs stimulated in vitro. PBMCs were treated with indicated doses of ivermectin and then stimulated by anti-CD3/CD28 mAbs, IL-1β and TGF-β for 48 hours. The supernatants were analyzed for IL-17 and IFN-γ by ELISA. One representative of three different experiments is shown.

FIG. 10. Inhibition of TNF-α, IL-6, and IL-23 secretion by ivermectin in LPS-induced human PBMCs. PBMCs were treated with indicated doses of ivermectin and then stimulated by LPS (1 mg/mL) for 48 hours. The supernatants were analyzed for TNF-α, IL-6, and IL-23 by ELISA. One representative of three different experiments is shown.

FIG. 11. Inhibition by ivermectin of IL-1β in TLR-induced human monocytes. Treatment of purified human monocytes with ivermectin reduced IL-1β in a dose-dependent manner. Human monocytes were isolated from peripheral blood and treated with indicated doses of ivermectin and then stimulated by LPS, Pam2CSK4 and Pam3CSK4 for 24 hours. The supernatants were analyzed for IL-1β by ELISA. Bar graphs represent pooled data from 2-3 experiments. Error bars, mean±S.D., *P≤0.05, **P≤0.01, ***P≤0.001, ****P≤0.0001.

FIG. 12. Ivermectin inhibits TNF-α and IL-6 secretion in dendritic cells. Treatment of monocytes-derived dendritic cells with ivermectin resulted in a dose dependent decrease in TNF-α and IL-6. Monocytes-derived dendritic cells were treated with indicated doses of ivermectin and then stimulated by Pam2CSK4 and Pam3CSK4 for 24 hours. The supernatants were analyzed for IL-6 and TNF-α by ELISA. Bar graphs represent pooled data from 2-3 experiments. Error bars, mean±S.D., *P≤0.05, **P≤0.01.

FIGS. 13A, 13B, 13C, 13D, 13E, 13F, and 13G illustrate the pharmacokinetic data (PK) obtained from the phase 1 clinical trial (single ascending dose (SAD) portion). Patients were administered the indicated dose of formulation 7E. FIG. 13G provides a summary of the data presented in FIGS. 13A, 13B, 13C, 13D, 13E, and 13F.

FIG. 14. Ivermectin down-regulates IL-17 secretion in PBMCs of human subjects stimulated ex vivo. PBMCs were purified from healthy human subjects before and after (24 hours after the last dosing) oral administration of ivermectin at doses 10, 20, 40, 80, 120 mg or placebo, and stimulated for 48 hours by immobilized monoclonal antibodies (mAbs) against CD3 and CD28 (anti-CD3/CD28). The supernatants were analyzed for IL-17 by ELISA. Statistical significance: treatment effect vs placebo *p<0.05, **p<0.01, ***p<0.001, **** p<0.0001 by Two-way RM ANOVA, Dunnett's multiple comparisons test.

FIGS. 15A and 15B illustrate efficacy of 7E to reduced seizures. FIG. 15A. Dose response trend of formulation 7E in reducing monthly focal seizure frequency. FIG. 15B. Responder analysis, which compared the proportion of study subjects treated with formulation 7E who achieved a ≥50% reduction in monthly focal seizures versus placebo.

FIGS. 16A, 16B, 16C, 16D, 116E, and 16F illustrate that oral administration of formulation 7E significantly down-regulates pro-inflammatory cytokine secretion in PBMCs after 12 weeks of treatment. FIG. 16A. IL-17, IL-21, IFN-γ, and TNF-α secretion in ex vivo stimulated PBMCs from patients with epilepsy on Day 1 before dosing begins (Pre-dose) and after 2, 4, 8, and 12 weeks of formulation 7E treatment (60 mg) or placebo (marked by *). The supernatants were collected 48 hours after stimulation by immobilized mAbs against CD3/CD28 and analyzed by ELISA. The numbers are indicative of average from duplicates for the same sample. Statistical significance: *p<0.05; **p<0.01 Two-way ANOVA; Fisher's LSD test. FIG. 16B illustrates cytokine secretion in ex vivo stimulated PBMCs from formulation 7E (60 mg) vs placebo treated patients with epilepsy. Out of the two bars for each time point, the left bar represents the placebo group and the right bar represents the treatment group. Percent change was calculated as (after-dosing value/pre-dosing −1)×100. Data is represented as Mean±SEM. Placebo n=2; formulation 7E n=4; at week 12 Placebo n=1; formulation 7E n=3. FIGS. 16C, 16D, 16E, and 16F show a summary of the plasma analysis results obtained for pro-inflammatory cytokine IL-17. Plasma samples: Placebo n=6/5; formulation 7E (60 mg) n=8/7. Statistical significance: *p<0.05; **p<0.01 Two-way ANOVA; Fisher's LSD test. FIG. 16C shows IL-17 release in plasma samples in response to treatment with 60 mg (once daily) formulation 7E. FIG. 16D shows % change in IL-17 secretion as compared to placebo for indicated treatment conditions. Out of the two bars for each time point, the left bar represents the placebo group and the right bar represents the treatment group. FIG. 16E shows % change in IL-17 secretion as compared to placebo for indicated treatment conditions. Bars correspond to the following groups from left to right (for each time point): Placebo, treatment (60 mg), treatment (40 mg), treatment (20 mg). FIG. 16F shows % patients with reduced IL-17 secretion in response to treatment with 60 mg (once daily) formulation 7E. Out of the two bars for each time point, the left bar represents the placebo group and the right bar represents the treatment group.

FIGS. 17A, 17B, 17C, 17D, and 17E illustrate secretion of pro-inflammatory cytokine in various patient groups. FIG. 17A illustrates pro-inflammatory cytokine secretion by PBMCs in healthy vs epileptic patients. n=12 healthy (NYBC); n=64 healthy (study described in Example 14); n=13 epileptic patients (study described in Example 16). Statistical significance: *p<0.05, **p<0.01, ***p<0.001, ****p<0.0001 Ordinary one-way ANOVA, Tukey's multiple comparisons test. FIGS. 17B, 17C, 17D, and 17E show a summary of the plasma analysis results obtained for pro-inflammatory cytokine IL-1β. Plasma samples: Placebo n=7/6; formulation 7E (60 mg) n=8/7. Statistical significance: *p<0.05; **p<0.01 Two-way ANOVA; Fisher's LSD test. FIG. 17B shows IL-1β release in plasma samples in response to treatment with 60 mg (once daily) formulation 7E. FIG. 17C shows % change in IL-1β secretion as compared to placebo for indicated treatment conditions (treatment once daily with formulation 7E). Out of the two bars for each time point, the left bar represents the placebo group and the right bar represents the treatment group. FIG. 17D shows % change in IL-1β secretion as compared to placebo for indicated treatment conditions (treatment once daily with formulation 7E). Bars correspond to the following groups from left to right (for each time point): Placebo, treatment (60 mg), treatment (40 mg), treatment (20 mg), treatment (10 mg). FIG. 17E shows % patients with reduced in IL-1β secretion in response to treatment with 60 mg (once daily) formulation 7E. Out of the two bars for each time point, the left bar represents the placebo group and the right bar represents the treatment group.

FIG. 18 illustrates changes (compared to pre-dose) in pro-inflammatory cytokine secretion in unstimulated PBMCs from formulation 7E (60 mg) vs placebo treated patients with epilepsy. Out of the two bars for each time point, the left bar represents the placebo group and the right bar represents the treatment group. % change was calculated as (after-dosing value/pre-dosing−1)×100. Data is represented as Mean±SEM. Placebo n=2; formulation 7E n=4; at week 12 Placebo n=1; formulation 7E n=3.

FIGS. 19A and 19B illustrate a correlation between changes in seizures frequency and T-cell cytokines in individual patients. FIG. 19A. Percent change over time. FIG. 19B. Correlation at eight weeks.

FIGS. 20A and 20B illustrate the percentage of Foxp3⁺/CD4⁺ in PBMCs isolated from patients receiving 7E treatment. FIG. 20A illustrates the percentage of Foxp3⁺/CD4⁺ in PBMCs purified from Cohort 4 patients with epilepsy on Day 1 before dosing begins (Pre-dose) and after 2, 4, 8, and 12 weeks of 7E treatment at 60 mg. Statistical significance: * p<0.05; **p<0.01, ***p<0.005 Two-way ANOVA; Fisher's LSD test. FIG. 20B illustrates the percentage of Foxp3⁺/CD4⁺ in PBMCs purified from Cohort 3 patients with epilepsy on Day 1 before dosing begins (Pre-dose) and after 2, 4, 8, and 12 weeks of 7E treatment at 40 mg. Statistical significance: *p<0.05; Paired t test.

FIGS. 21A, 21B, and 21C illustrate that ivermectin significantly downregulates limb paralysis in an experimental autoimmune encephalomyelitis (EAE) model of MS. FIG. 21A. Clinical scores were assessed as follows: 1=limp tail, 2=partial hind leg paralysis, 3=complete hind leg paralysis, 4=complete hindleg and partial front leg paralysis; 5=moribund. 1, 2, 4 refer to mg/kg of ivermectin. FIG. 21B. Bars from left to right: Vehicle, 4 mg/kg ivermectin, FTY720 (fingolimod). FIG. 21C. Bars from left to right: Vehicle, 4 mg/kg ivermectin, FTY720.

FIGS. 22A, FIGS. 22B, and 22C show data from an experiment using ivermectin in a Traumatic Brain Injury (TBI) model. FIG. 22A illustrates the experimental setup. FIG. 22B shows the latency and path length for the mouse to find the platform. Individual change as compared to the performance level at day −4 (last day of pre-testing). Data are presented as mean+SEM, n=11-15 per group. Bars from left to right: Sham; TBI; TBI+ivermectin. FIG. 22C shows secretion of inflammatory cytokines after TBI (left bars) or TBI+ivermectin treatment (right bars).

FIGS. 23A and 23B show data from an experiment using ivermectin for the treatment for infantile spasms. FIG. 23A shows the experimental setup. G=gestational day. P=postnatal day. Spasms were induced by i.p. injection of N-methyl-D-aspartic acid (NMDA). FIG. 23B. Effects of ivermectin in repeated administration (P10-P15) on the number of spasms triggered on P15. Groups treated with 2 and 4 mg/kg of ivermectin had significantly fewer spasms than the control group injected with vehicle (*p<0.05). 1, 2, 4 refer to mg/kg ivermectin.

### DETAILED DESCRIPTION

The present disclosure provides pharmaceutical compositions and dosage forms of avermectins, and particularly of ivermectin. The compositions (also referred herein interchangeably as formulations) of the avermectins are liquid or semi-solid at room temperature and comprise an avermectin, and particularly ivermectin, a first surfactant and a second surfactant. The compositions may be used for the treatment and control of various inflammatory disorders in humans, including neuroinflammatory and autoimmune disorders.

### Avermectins and Ivermectin

Any of the avermectin compounds, which includes derivatives and analogs disclosed herein, may be used in the compositions and methods provided by this disclosure. Avermectins include a family of four closely related major components, A1a, A2a, B1a and B2a and four minor components A1b, A2b, B1b, B2b which are lower homologs of the corresponding major components. Eight different avermectins were isolated in four pairs of homologue compounds, with a major and minor component usually in ratios of about 80:20 to about 90:10. Anthelmintics derived from the avermectins include ivermectin, selamectin, doramectin, eprinomectin, moxidectin, avermectin, and abamectin. The family members show anthelmintic and insecticidal/acaricidal activity in different degree of potencies.

Provided herein are compositions comprising about 1% to about 15% of an avermectin compound, wherein the avermectin compound may be a macrocyclic compound according to Formula I:

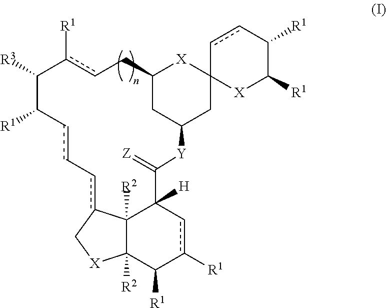

or a pharmaceutically acceptable salt, stereoisomer, polymorph, solvate, or isotopically labeled compound thereof, wherein:
each occurrence of X is independently selected from —CH₂—, —NH—, —O—, —S—, —SO— and —SO₂—;
Y is selected from —CH₂—, —O—, —NH—, and —S—;
Z is selected from O and S;
each occurrence of [symbol] is a single bond or a double bond;
n is an integer 0-6; and
each occurrence of R¹ is independently selected from halogen, —R, —OR, —NO₂, —NCS, —CN, —CF₃, —OCF₃, —NHR, —N(R)₂, —OC(O)R, —C(O)OR, —SR, —C(O)R, —C(O)C(O)R, —C(O)CH₂C(O)R, —C(S)R, —C(S)OR, —C(O)C(O)OR, —C(O)C(O)N(R)₂, —C(O)N(R)₂, —OC(O)N(R)₂, —C(S)N(R)₂, —N(R)C(O)OR, —N(R)C(O)R, —N(R)C(S)R, —N(R)C(O)N(R)₂, —N(R)C(S)N(R)₂, —N(COR)COR, —N(OR)R, —C(═NH)N(R)₂, —C(O)N(OR)R, —C(═NOR)R, —OP(O)(OR)₂, —P(O)(R)₂, —P(O)(OR)₂, —P(O)(H)(OR), —CH₂—OR, and —CH₂—O—CH₂—R;
each occurrence of R² is independently selected from independently selected from H, OH, O—C₁₋₄alkyl, —OC(O)C₁₋₄alkyl, —OC(O)NH₂, and —OC(O)NHC₁₋₄alkyl;
each occurrence of R³ is mono, di, or triglycoside, or OC(O)—(C₃-C₅)alkenyl;
each R is independently selected from H, —(C₁-C₁₂)alkyl, —(C₃-C₁₀)-cycloalkyl, (C₃-C₁₀)-cycloalkenyl, —[(C₃-C₁₀)cycloalkyl]-(C₁-C₁₂)alkyl, —[(C₃-C₁₀)cycloalkenyl]-(C₁-C₁₂)alkyl, —[(C₃-C₁₀)cycloalkenyl]-(C₁-C₁₂)alkyl, —[(C₃-C₁₀)cycloalkyl]-O—(C₁-C₁₂)alkyl, —[(C₃-C₁₀)cycloalkenyl]-O—(C₁-C₁₂)alkyl, —(C₆-C₁₀)aryl, (C₆-C₁₀)aryl-(C₁-C₁₂)alkyl, —(C₆-C₁₀)aryl-O—(C₁-C₁₂)alkyl, (C₆-C₁₀)aryl-N(R″)—(C₁-C₁₂)alkyl, 3- to 10-membered heterocyclyl, (3- to 10-membered heterocyclyl)-(C₁-C₁₂)alkyl, (3- to 10-membered heterocyclyl)-O—(C₁-C₁₂)alkyl, (3- to 10-membered heterocyclyl)-N(R″)—(C₁-C₁₂)alkyl, 5- to 10-membered heteroaryl, (5- to 10-membered heteroaryl)-(C₁-C₁₂)-alkyl, (5- to 10-membered heteroaryl)-O—(C₁-C₁₂)-alkyl and (5- to 10-membered heteroaryl)-N(R″)—(C₁-C₁₂)-alkyl;
   each heterocyclyl has 1-4 heteroatoms independently selected from N, NH, O, S, SO, and SO₂, and
   heteroaryl has 1-4 heteroatoms independently selected from N, NH, O, and S;
each occurrence of R is independently unsubstituted or is substituted with 1 to 5 R′;
each occurrence of R′ is halo, OH, oxo, —CH₂OR″, —CH₂N(R″)₂, —C(O)N(R″)₂, —C(O)OR″, —NO₂, —NCS, —CN, —CF₃, —OCF₃ and —N(R″)₂; and
each occurrence of R″ is independently H, C₁₋₆alkyl, C₂₋₆alkenyl, C₃₋₆cycloalkyl, C₃₋₆ cycloalkenyl, 3- to 6-membered heterocyclyl, 5- to 10-membered heteroaryl, and (C₆-C₁₀)-aryl.

In some embodiments, the compound of Formula I is a compound of Formula II:

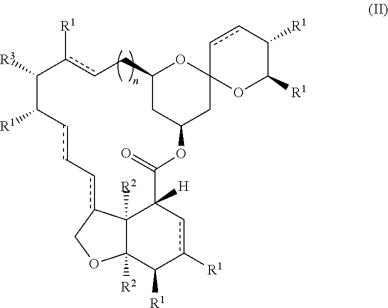

or a pharmaceutically acceptable salt, stereoisomer, polymorph, solvate or isotopically labeled compound thereof, wherein n, R¹, R², and R³ are each as defined in Formula I.

In some embodiments, the compound of Formula II is a compound of Formula III:

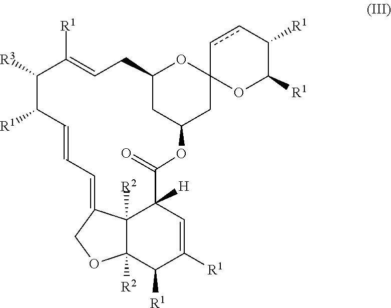

or a pharmaceutically acceptable salt, stereoisomer, polymorph, solvate or isotopically labeled compound thereof, wherein R¹, R², and R³ are each as defined in Formula I.

In some embodiments, the compound of Formula III is a compound of Formula IV:

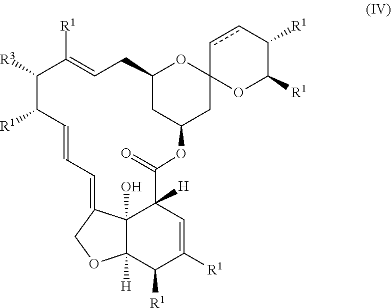

or a pharmaceutically acceptable salt, stereoisomer, polymorph, solvate or isotopically labeled compound thereof, wherein:
each occurrence of R¹ is independently selected from halogen, —R, —OR, —NO₂, —NCS, —CN, —CF₃, —OCF₃, —NHR, —N(R)₂, —OC(O)R, —C(O)OR, —C(O)N(R)₂, —OC(O)N(R)₂, —N(R)C(O)OR, —N(R)C(O)R, —N(R)C(O)N(R)₂, —CH₂—OR, and —CH₂—O—CH₂—R;
each occurrence of R³ is mono, di, or triglycoside, or OC(O)—(C₃-C₅) alkenyl; R, R′ and R″ are each as defined in Formula I.

In some embodiments, the compound of Formula IV is a compound of Formula V:

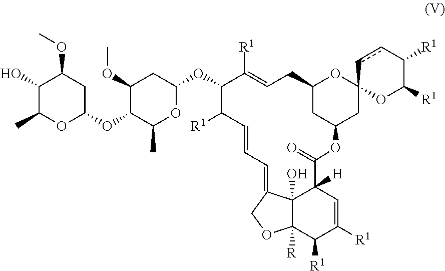

or a pharmaceutically acceptable salt, stereoisomer, polymorph, solvate or isotopically labeled compound thereof, wherein
each occurrence of R¹ is independently selected from halogen, —R, —OR, —NO₂, —NCS, —CN, —CF₃, —OCF₃, —NHR, —N(R)₂, —OC(O)R, —C(O)OR, —C(O)N(R)₂, —OC(O)N(R)₂, —N(R)C(O)OR, —N(R)C(O)R, —N(R)C(O)N(R)₂, —CH₂—OR, and —CH₂—O—CH₂—R;
R, R′ and R″ are each as defined in Formula I.

In embodiments, the macrocyclic compound is an avermectin compound or derivative, a pharmaceutically acceptable salt, ester, stereoisomer, polymorph, solvate, N-oxide, or isotopically labeled compound thereof. In an embodiment, the macrocyclic compound is avermectin. In some embodiments, the macrocyclic compound is selected from the group consisting of ivermectin, selamectin, doramectin, eprinomectin, moxidectin, avermectin, and abamectin. In some embodiments, the macrocyclic compound is ivermectin or a derivative, a pharmaceutically acceptable salt, ester, stereoisomer, polymorph, solvate, N-oxide, or isotopically labeled compound thereof.

Provided herein is a composition comprising about 1% to about 15% of a compound of any one of Formulas I-VII. In some embodiments, the composition comprises about 1% to about 15% of one or more of a compound of any one of Formulas I-VII. In some embodiments, the composition comprises about 1% to about 15% ivermectin comprising 22,23-dihydroavermectin B1a (Formula VI) and 22,23-dihydroavermectin Bib (Formula VII). In some embodiments, the composition comprises ivermectin comprising from at least about 70% to at least about 90% of a compound of Formula VI and from less than about 30% to less than about 10% of a compound of Formula VII:

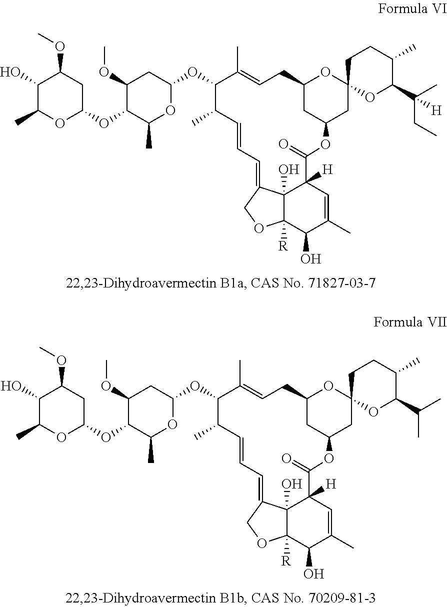

In some embodiments, the composition comprises ivermectin comprising from at least about 70% to at least about 90% of a compound of Formula VI and from less than about 30% to less than about 10% of a compound of Formula VII. In some embodiments, ivermectin comprises at least about 70% of 22,23-dihydroavermectin B1a and less than about 30% of 22,23-dihydroavermectin B1b. In some embodiments, ivermectin comprises at least about 90% of 22,23-dihydroavermectin B1a and less than about 10% of 22,23-dihydroavermectin B1b.

Derivatives of ivermectin, including abamectin and doramectin, and prodrugs of ivermectin, may have properties and uses similar to those of ivermectin. Abamectin and doramectin both have a double bond at positions C22-C23 in the structural formula of ivermectin. Additionally, in doramectin, position C25 is substituted at the side chain of a cyclohexyl ring.

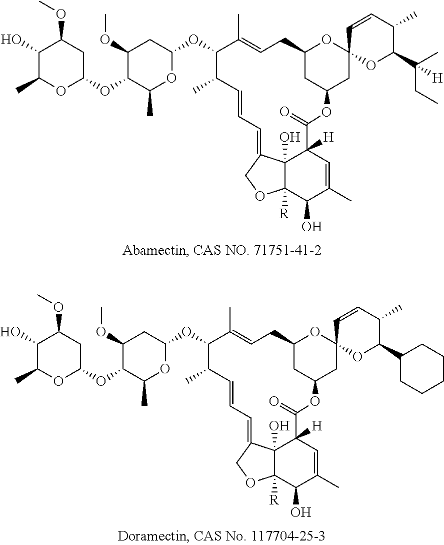

As used herein, “derivative” to a compound that retains the biological activity of the parent avermectin from which it is derived or is a prodrug for the parent avermectin. Derivatives may include esters, amides, ethers or the like that are derived from the avermectin.

As used herein, the term “alkyl” refers to a hydrocarbon chain that is a straight chain or a branched chain, containing the indicated number of carbon atoms. For example, C₁₋₆ indicates that the group has from 1 to 6 (inclusive) carbon atoms in it. In the absence of any numerical designation, “alkyl” is a chain (straight or branched) having 1 to 20 (inclusive) carbon atoms in it. The term “unsaturated alkyl” refers to a hydrocarbon having no unsaturated bonds (e.g., a carbon-carbon double bond or a carbon-carbon triple bond). The term “unsaturated alkyl” refers to a hydrocarbon having one or more unsaturated bonds (e.g., a carbon-carbon double bond or a carbon-carbon triple bond).

As used herein, the term “alkenyl” refers to a hydrocarbon chain that is a straight chain or branched chain having one or more carbon-carbon double bonds. The alkenyl moiety contains the indicated number of carbon atoms. For example, C₂₋₆ indicates that the group has from 2 to 6 (inclusive) carbon atoms in it. In the absence of any numerical designation, “alkenyl” is a chain (straight or branched) having 2 to 20 (inclusive) carbon atoms in it.

The term “cycloalkyl” as employed herein includes saturated and partially unsaturated cyclic hydrocarbon groups. Unless indicated otherwise, a cycloalkyl has 3 to 12 carbons, or 3 to 8 carbons, or 3 to 6 carbons, wherein the cycloalkyl group additionally is optionally substituted. Some cycloalkyl groups include, without limitation, cyclopropyl, cyclobutyl, cyclopentyl, cyclopentenyl, cyclohexyl, cyclohexenyl, cycloheptyl, and cyclooctyl.

As used herein, the term “aryl” refers to a 6 to 10 carbon monocyclic or bicyclic aromatic ring system wherein 0, 1, 2, 3, or 4 atoms of each ring are substituted by a substituent. Examples of aryl groups include phenyl, naphthyl and the like.

As used herein, the term “heterocyclyl group” or “heterocyclyl” refers to aromatic and non-aromatic ring compounds containing 3 or more ring members, of which, one or more is a heteroatom such as, but not limited to, N, O, and S. A heterocyclyl can be a cycloheteroalkyl, or a heteroaryl, or if polycyclic, any combination thereof. Heterocyclyl groups may comprise from 3 to about 20 ring members, whereas other such groups may comprise from 3 to about 15 ring members. A heterocyclyl group designated as a C₂-heterocyclyl can be a 5-membered ring with two carbon atoms and three heteroatoms, a 6-membered ring with two carbon atoms and four heteroatoms, and so forth. Likewise, a C₄-heterocyclyl can be a 5-membered ring with one heteroatom, a 6-membered ring with two heteroatoms, and so forth. The number of carbon atoms and the number of heteroatoms, when summed up, equal the total number of ring atoms. A heterocyclyl ring can also include one or more double bonds. A heteroaryl ring is an embodiment of a heterocyclyl group. In some embodiment, the “heterocyclyl” refers to a nonaromatic 5-8 membered monocyclic, 8-12 membered bicyclic, or 11-14 membered tricyclic ring system having 1-3 heteroatoms if monocyclic, 1-6 heteroatoms if bicyclic, or 1-9 heteroatoms if tricyclic, said heteroatoms selected from O, N, or S (e.g., carbon atoms and 1-3, 1-6, or 1-9 heteroatoms of O, N, or S if monocyclic, bicyclic, or tricyclic, respectively), wherein 0, 1, 2 or 3 atoms of each ring are substituted by a substituent. Examples of heterocyclyl groups include piperazinyl, pyrrolidinyl, dioxanyl, morpholinyl, tetrahydrofuranyl, and the like.

As used herein, the term “heteroaryl” refers to an aromatic 5-8 membered monocyclic, 8-12 membered bicyclic, or 11-14 membered tricyclic ring system having 1-3 heteroatoms if monocyclic, 1-6 heteroatoms if bicyclic, or 1-9 heteroatoms if tricyclic, said heteroatoms selected from O, N, or S (e.g., carbon atoms and 1-3, 1-6, or 1-9 heteroatoms of O, N, or S if monocyclic, bicyclic, or tricyclic, respectively), wherein 0, 1, 2, 3, or 4 atoms of each ring are substituted by a substituent. Examples of heteroaryl groups include pyridyl, furyl or furanyl, imidazolyl, benzimidazolyl, pyrimidinyl, thiophenyl or thienyl, quinolinyl, indolyl, thiazolyl, and the like.

As used herein, the term “glycoside” refers to any material with a chemical structure comprising a glycosidic bond between a carbohydrate (sugar) molecule and another carbohydrate or a non-carbohydrate (non-sugar) moiety. A glycosidic bond or glycosidic linkage is a type of covalent bond that joins a carbohydrate (sugar) molecule, for example, via its hemiacetal or hemiketal group, to another molecule. The other molecule may or may not be a carbohydrate. The sugar moiety is generally known as the glycone part of a glycoside. The glycone can consist of a single sugar group (monosaccharide), two sugar groups (disaccharide) or several sugar groups (oligosaccharide).

As used herein, the term “substituent” refers to a group replacing a second atom or group such as a hydrogen atom on any molecule, compound or moiety. Suitable substituents include, without limitation, halo, hydroxy, mercapto, oxo, nitro, haloalkyl, alkyl, alkaryl, aryl, aralkyl, alkoxy, thioalkoxy, aryloxy, amino, alkoxycarbonyl, amido, carboxy, alkanesulfonyl, alkylcarbonyl, and cyano groups.

In embodiments, the avermectin used in the formulations and methods provided herein will be in the neutral form. However, when the avermectin comprises an ionizable group, the avermectin may be present as a pharmaceutically acceptable salt. As used herein, the term “pharmaceutically acceptable salt” refers to those salts which are suitable for pharmaceutical use, such as, for example, for use in humans and animals. Pharmaceutically acceptable salts of amines, carboxylic acids, and other types of compounds, are well known in the art. For example, S. M. Berge, et al., describe pharmaceutically acceptable salts in detail in J Pharmaceutical Sciences, 66: 1-19 (1977), incorporated herein by reference. The salts can be prepared in situ during the final isolation and purification of the avermectin, or separately by reacting a free base or free acid function with a suitable reagent. For example, a free base function can be reacted with a suitable acid. Suitable pharmaceutically acceptable salts can, include metal salts such as alkali metal salts, e. g. sodium, potassium, and lithium salts; and alkaline earth metal salts, e. g. calcium or magnesium salts. Examples of pharmaceutically acceptable, nontoxic acid addition salts are salts of an amino group formed with inorganic acids such as hydrochloric acid, hydrobromic acid, hydroiodic acid, phosphoric acid, sulfuric acid and perchloric acid or with organic acids such as acetic acid, oxalic acid, maleic acid, tartaric acid, citric acid, succinic acid or malonic acid or by using other methods used in the art such as ion exchange. Other pharmaceutically acceptable salts include adipate, alginate, ascorbate, aspartate, benzoate, bisulfate, borate, butyrate, camphorate, camphorsulfonate, citrate, cyclopentanepropionate, digluconate, dodecylsulfate, formate, fumarate, glucoheptonate, glycerophosphate, gluconate, hernisulfate, heptanoate, hexanoate, hydroiodide, 2-hydroxy-ethanesulfonate, lactobionate, lactate, laurate, lauryl sulfate, malate, maleate, malonate, methanesulfonate, nicotinate, nitrate, oleate, oxalate, palmitate, pectinate, persulfate, 3-phenylpropionate, phosphate, picrate, pivalate, propionate, stearate, succinate, sulfate, tartrate, thiocyanate, p-toluenesulfonate, undecanoate, valerate salts, and the like.

### Avermectin Compositions

The compositions for use in the methods and dosages provided herein are liquid or semi-solid at room temperature and comprise an avermectin, and particularly ivermectin, a first surfactant and a second surfactant.

In embodiments, the composition comprises:
(i) about 1% to about 15% of a compound of any one of Formulas I-VII;
(ii) about 20% to about 40% of a first surfactant comprising one or more of:
   (a) mono-, di-, and/or tri-fatty acid esters of glycerol;
   (b) mono- and/or di-fatty acid esters of 1,2-propylene glycol; and
   (c) mono- and/or di-fatty acid esters of polyethylene glycol;
   wherein the fatty acids are selected from C₆ to C₁₀ fatty acids; and
(iii) about 15% to about 70% of a second surfactant selected from one or more of a polysorbate surfactant and/or a fatty acid ester of sorbitan.

In some embodiments, the composition comprises about 3% to about 12% of a compound of any one of Formulas I-VII. In some embodiments, the composition comprises about 5% to about 10% of a compound of any one of Formulas I-VII. In some embodiments, the composition comprises about 1%, about 2%, about 3%, about 4%, about 5%, about 6%, about 7%, about 8%, about 9%, about 10%, about 11%, or about 12% of a compound of any one of Formulas I-VII.

In embodiments, the composition comprises:
(i) about 1% to about 15% of one or more of a compound of any one of Formulas I-VII;
(ii) about 20% to about 40% of a first surfactant comprising one or more of:
   (a) mono-, di-, and/or tri-fatty acid esters of glycerol;
   (b) mono- and/or di-fatty acid esters of 1,2-propylene glycol; and
   (c) mono- and/or di-fatty acid esters of polyethylene glycol;
   wherein the fatty acids are selected from C₆ to C₁₀ fatty acids; and
(iii) about 15% to about 70% of a second surfactant selected from one or more of a polysorbate surfactant and/or a fatty acid ester of sorbitan.

In some embodiments, the composition comprises about 3% to about 12% of one or more of a compound of Formulas I-VII. In some embodiments, the composition comprises about 5% to about 10% of one or more of a of Formulas I-VII. In some embodiments, the composition comprises about 1%, about 2%, about 3%, about 4%, about 5%, about 6%, about 7%, about 8%, about 9%, about 10%, about 11%, or about 12% of one or more of a of Formulas I-VII.

In embodiments, the composition comprises:
(i) about 1% to about 15% of ivermectin comprising a compound of Formula VI and a compound of Formula VII;
(ii) about 20% to about 40% of a first surfactant comprising one or more of:
   (a) mono-, di-, and/or tri-fatty acid esters of glycerol;
   (b) mono- and/or di-fatty acid esters of 1,2-propylene glycol; and
   (c) mono- and/or di-fatty acid esters of polyethylene glycol;
   wherein the fatty acids are selected from C₆ to C₁₀ fatty acids; and
(iii) about 15% to about 70% of a second surfactant selected from one or more of a polysorbate surfactant and/or a fatty acid ester of sorbitan.

In some embodiments, the composition comprises about 3% to about 12% of ivermectin comprising a compound of Formula VI and a compound of Formula VII. In some embodiments, the composition comprises about 5% to about 10% ivermectin comprising a compound of Formula VI and a compound of Formula VII. In some embodiments, the composition comprises about 1%, about 2%, about 3%, about 4%, about 5%, about 6%, about 7%, about 8%, about 9%, about 10%, about 11%, or about 12% ivermectin comprising a compound of Formula VI and a compound of Formula VII.

The composition provided herein comprises a first surfactant, which may be a mixture of surfactants, and comprise C₆ to C₁₀ fatty acid esters of glycerol, of 1,2-propylene glycol, and/or of polyethylene glycol. The C₆ to C₁₀ fatty acid(s) may be saturated or unsaturated, but is preferably saturated. More preferably, the fatty acid has from 8 to 10 carbons, and particularly may be caprate, caprylate and mixtures thereof. The avermectin composition comprises about 20% to about 40% of the first surfactant, or about 25% to about 30% of the first surfactant.

The first surfactant may comprise mono-, di-, and/or tri-fatty acid esters of glycerol. The fatty acid ester of glycerol may be a mixture of mono- and di-esters of glycerol. The fatty acid component is one or more C₆ to C₁₀ fatty acid(s) and may be saturated or unsaturated, but is preferable saturated. More preferably, the fatty acid has from 8 to 10 carbons, and particularly may be caprate, caprylate and mixtures thereof. The fatty acid esters of glycerol are commercially available and include Capmul 808G EP/NF (glyceryl monocaprylate), Capmul MCM C8 EP/NF (glyceryl monocaprylate), Capmul MCM (glyceryl caprylate/caprate), Masester E8120 (glycerol mono- and di-caprylate/caprate).

Additionally, or alternatively, the first surfactant may comprise mono- and/or di-fatty acid esters of 1,2-propylene glycol. The fatty acid esters of propylene glycol may be a mixture of mono- and di-esters of propylene glycol, and preferably the fatty acid ester of propylene glycol may be a mono-ester of propylene glycol. The fatty acid component is one or more C₆ to C₁₀ fatty acid(s) and may be saturated or unsaturated but is preferable saturated. More preferably, the fatty acid has from 8 to 10 carbons, and particularly may be caprate, caprylate and mixtures thereof. The fatty acid esters of propylene glycol are commercially available and include Capmul PG-8 (propylene glycol monocaprylate), Capryol 90 (propylene glycol monocaprylate), Capryol PGMC (propylene glycol mono- and di-caprylate).

The first surfactant may additionally or alternatively comprise mono- and/or di-fatty acid esters of polyethylene glycol. The fatty acid ester of polyethylene glycol may be a mixture of mono- and di-esters of polyethylene glycol. The fatty acid component is one or more C₆ to C₁₀ fatty acid(s) and may be saturated or unsaturated but is preferable saturated. More preferably, the fatty acid has from 8 to 10 carbons, and particularly may be caprate, caprylate and mixtures thereof. The polyethylene glycol component may have an average molecular weight of from about 200 to about 800, or from about 300 to about 500. The fatty acid esters of propylene glycol are commercially available and include Labrasol ALF (small fraction of mono-, di- and triglycerides and mainly PEG-8 (MW 400) mono- and diesters of caprylic (C₈) and capric (C₁₀) acids).

The compositions provided herein also comprise a second surfactant, which may be a mixture of surfactants, and is selected from polysorbate surfactants, fatty acid esters of sorbitan, and mixtures thereof. The avermectin composition comprises about 15% to about 70% of a second surfactant, about 15% to about 40% of a second surfactant, or about 15% to about 20% of the second surfactant.

The second surfactant may comprise polysorbate surfactants. The polysorbate surfactants are polyethoxylated sorbitan esterified with fatty acids having the following general structure:

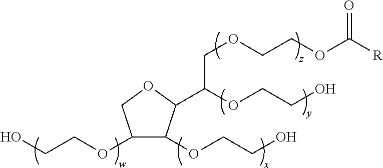

in which R is the carbon chain of a medium to long chain fatty acid, and the sum of w, x, y, and z is the number of oxyethylene —(CH₂CH₂O)— groups found in the molecule. The fatty acid may be saturated or unsaturated. In embodiments, the fatty acid has from 12 to 18 carbons. The number of oxyethylene groups (i.e., w+x+y+z) is in embodiments about 20. Polysorbate surfactants are commercially available and include polysorbate 80 (polyoxyethylene 20 sorbitan monooleate) such as Tween 80, Montanox 80, Alkest TW 80; polysorbate 60 (polyoxyethylene 20 sorbitan monostearate) such as Tween 60; polysorbate 40 (polyoxyethylene 20 sorbitan monopalmitate) such as Tween 40; and polysorbate 20 (polyoxyethylene 20 sorbitan monolaurate) such as Tween 20 and Alkest TW 20.

The second surfactant may additionally or alternatively comprise fatty acid esters of sorbitan (also known as Spans). The fatty acid component may be saturated or unsaturated. In embodiments, the fatty acid has from 12 to 18 carbons. The sorbitan ester may be mono-, di-or tri-esters of sorbitan, and particularly are mono-esters of sorbitan, such as sorbitan monolaurate. Sorbitan esters are commercially available and include Span 20 (sorbitan monolaurate), Span 40 (sorbitan monopalmitate), Span 60 (sorbitan monostearate), Span 65 (sorbitan tristearate) and Span 80 (sorbitan monooleate).

Optionally, the avermectin composition may additionally comprise vitamin E TPGS, also known as D-α-Tocopherol polyethylene glycol 1000 succinate. The composition may comprise from about 5% to about 55% vitamin E TPGS, or from about 30% to about 50% vitamin E TPGS.

In embodiments, the pharmaceutical composition comprises: (i) about 5% to about 10% ivermectin; (ii) about 25% to about 30% mono- and di- C₈ to C₁₀ fatty acid esters of glycerol; (iii) about 15% to about 30% of a polysorbate surfactant; and (iv) about 30% to about 50% vitamin E TPGS.

In an embodiment, the pharmaceutical composition comprises: (i) about 8% ivermectin; (ii) about 27.6% mono- and di- C₈ to C₁₀ fatty acid esters of glycerol; (iii) about 32.2% polysorbate 80; and (iv) about 32.2% vitamin E TPGS.

In an embodiment, the pharmaceutical composition comprises: (i) about 8% ivermectin; (ii) about 27.6% Masester E8120; (iii) about 32.2% polysorbate 80; and (iv) about 32.2% vitamin E TPGs.

In an embodiment, the pharmaceutical composition comprises: (i) about 8% ivermectin; (ii) about 27.6% mono- and di- C₈ to C₁₀ fatty acid esters of glycerol; (iii) about 18.4% polysorbate 80; and (iv) about 46% vitamin E TPGS.

In an embodiment, the pharmaceutical composition comprises: (i) about 8% ivermectin; (ii) about 27.6% Masester E8120; (iii) about 18.4% polysorbate 80; and (iv) about 46% vitamin E TPGS.

In an embodiment, the pharmaceutical composition comprises: (i) about 8% ivermectin; (ii) about 27.6% mono- and di-C₈ to C₁₀ fatty acid esters of glycerol; and (iii) about 64.4% polysorbate 80.

In embodiments, the pharmaceutical composition comprises: (i) about 8% ivermectin; (ii) about 27.6% Masester E8120; and (iii) about 64.4% polysorbate 80.

The pharmaceutical composition may additionally comprise additional excipients such as additional surfactants, solvents, solubilizing agents, preservatives, anti-oxidants, bulking agents, dissolution enhancers, wetting agents, emulsifiers, suspending agents, antibacterial agents, pH buffering agents, sweeteners, flavoring agents, and combinations thereof.

### Dosage Form

The disclosure provides a composition comprising the avermectin is a semi-solid or liquid-based when formulated as, including, but not limited to, an emulsion, suspension, solution, elixirs, or syrup in which the avermectin is dissolved and/or suspended.

The dosage form comprising the avermectin can take the form of solutions, suspensions, emulsion, aerosols, capsules, soft elastic or hard gelatin capsules, dermal patch, suspensions, and the like preferably in unit dosage forms suitable for simple administration of precise dosages. The composition may take the forms of liquid- or semi-solid filled capsules, sublingual spray, or nasal spray.

Capsule dosage forms may include soft capsules and hard capsules. Capsules may be used as an oral dosage form for the administration of many different types of active pharmaceuticals. The capsules may be filled with an active ingredient in the form of a liquid, or a powder suspended in liquid. Hard capsules can be made of unplasticized or low-plasticized gelatin and water to form a stiff capsule that can be filled with either powder or liquid. Soft capsules can be made of highly plasticized soft elastic gelatin and can contain a liquid or semisolid ingredient. These capsules are often referred to as “softgel” or “gelcap” capsules.

As used herein, the term “capsule” refers to any suitable capsular container or case adapted for oral ingestion, e.g., those adapted for use in conjunction with liquid fill compositions. The term “capsule” may include capsules having a shell composed of soft and/or hard materials, such as gelatin, starches, celluloses, cellulose derivatives (e.g., hydroxypropyl methyl cellulose), hydrocolloids, gums, carrageenans, or any other natural or synthetic material which can be used to encapsulate the liquid composition and be ingested by an animal. Optionally, the shell material can be gelatin and/or hydroxypropyl methyl cellulose. In an embodiment, the shell material is gelatin. The term “capsule” also includes a variety of capsule shapes and sizes. The instant disclosure does not limit the dosage form to a specific type or shape. Any commercially available capsule shells or shell materials can be used.

In an embodiment, the dosage form of the instant disclosure is a soft capsule. In an embodiment, the dosage form of the instant disclosure is a coated liquid-filled soft capsule. The coated capsule can include a liquid fill encapsulated with a soft capsule shell. The exterior surface of the soft capsule shell can be coated with one or more layers of coating.

Suitable materials for encapsulating the liquid fill may include heat sealable polymers and gelatin. Examples of heat sealable polymers may include, but are not limited to, modified starches, cellulosic polymers and carrageenans. In an embodiment, the material is gelatin. The gelatin can be natural gelatin, chemically modified gelatin, enzymatically modified gelatin, or combinations thereof.

The material that forms the capsule shell can further includes water. Water can be present in the original material mass before the capsules are made, in an amount sufficient to allow the processing of the material on the encapsulation machine. After the capsules are formed the majority of the moisture can be removed during the drying process.

The water can have a plasticizing effect on the material. In addition, a non-volatile plasticizer or blend of plasticizers can be added to the material which forms the capsule shell. The non-volatile plasticizer can be any plasticizer compatible with the material of the capsule shell. For example, the non-volatile plasticizer can be glycerin, maltitol, sorbitan, sorbitol or similar low molecular weight polyhydric alcohols, and mixtures thereof. In embodiments, the ratio of plasticizer to material determines how hard or soft the shell can be.

The ratio of plasticizer to material in the shell may be sufficient to provide capsules that are not too hard, such that the capsules are brittle and crack if stressed during shipping and handling, and are not too soft, such that the capsules become deformed during shipping and handling. The non-volatile plasticizer can be present in the capsule shell from about 8% to 65% by total weight of the capsule shell, from about 10% to 35% by total weight of the capsule shell.

The material which forms the capsule shell can further contain extenders. The extender can be any extender which is compatible with the material. Examples of extenders may include natural or modified natural biopolymers and synthetic polymers. Natural biopolymers may include, for instance, cellulose, starch, starch derivatives, bacterial polysaccharides such as xanthan gum and gellan gum and vegetable gums such as guar gum, locust bean gum, gum tragacanth and gum Arabic and animal derived polymers such as chondroitin sulfate, hyaluronic acid, heparin, collagen and chitosan. An example of a modified natural biopolymer may be modified cellulose. Examples of synthetic polymers may include carbon chain polymers of the vinyl and acrylic types as well as heterochains of the polyoxide and polyamine types.

A coating can be applied on the exterior surface of the soft capsule shell. The coating can contain one or more layers. Any coating suitable for a soft capsule can be applied to the capsule. The coating can provide, for example, waterproofing and sealing, smoothing, polishing, enteric protection and/or delayed release properties to the liquid-filled capsule. The delayed release can be affected by, for example, temperature or pH. In an embodiment, the coating is an enteric coating.

The coating can be made by any standard coating ingredient known to those skilled in the art. Coating ingredients may include, but are not limited to, fats, fatty acids, waxes, shellac, ammoniated shellac, cellulose acetate phyhalates, celluosics, vinyls, glycols, acrylics and carbohydrate polymers, polymers and co-polymers containing methacrylic acid and methacrylic acid alkyl esters, hydroxypropylmethyl cellulose (HPMC) and combinations thereof.

The coated capsule can further comprise a finishing coating. In an embodiment, the finishing layer is applied to the coated-capsule. Examples of substance suitable for use in a finishing coating may include, but are not limited to cellulosics, vinyls, glycols, acrylics and carbohydrate polymers and/or combinations thereof.

The liquid fill or semi-solid fill can be encapsulated with a soft capsule shell by any method known in the art. For example, a soft capsule can be made using a standard rotary die soft gelatin capsule machine as described in The Theory and Practice of Industrial Pharmacy, ed. Lachman, et al., 2nd Ed., Pt. II, 404-420, Lea & Febiger, 1976. Additional methods include using a plate process (see The Theory and Practice of Industrial Pharmacy, ed. Lachman, et al., 2nd Ed., Pt. II, 405, Lea & Febiger, 1976), as well as Globex type seamless capsule machines, which makes large microcapsules (see U.S. Pat. No. 5,254,294), non-standard rotary die machines, which uses extrusion technology to make gel ribbons (see U.S. Pat. Nos. 6,183,845 and 6,340,473), and other methods for making capsules which use high frequency, ultrasonic, or induction welding to seal the capsules (see U.S. Pat. No. 6,352,719). The above-listed U.S. patents and book are hereby incorporated by reference.

As used herein, the phrase “liquid hard-shell” refers to a hard capsule encapsulating a liquid or semi-solid formulation. Hard capsules can be single unit dosage forms and may comprise a cap and a body, which can be manufactured separately, and which can be supplied empty for filling with the liquid or semi-solid composition. In some embodiments, hard capsules are made from a polymer such as gelatin. An additional component can be water, which acts as a plasticizer. Another hard capsule may be manufactured from hydroxypropylmethyl cellulose (HPMC). Liquid-fill hard capsule can be filled on a filling machine, such as, for example, a high-speed filling machine.

In one example, disclosed herein is a method of prepare the filled hard capsule. Empty capsules are supplied to the filling machine in a prelocked condition, wherein the capsule body has a cap which is loosely attached thereto. A series of rings or protrusions are provided in the mating surfaces of the cap or body. These rings are configured to enable the cap to be loosely attached to the body so that the cap and body are held together during storage but would enable the cap to be removed prior to filling of the capsule. Once the capsule has been filled, the cap can be replaced and be forced beyond the prelocked position into a fully locked position. Alternatively, other types of capsule filling machines can be used to accept separate supplies of capsule bodies and caps.

The capsules may be closed at high speed after filling with the formulated composition. During closure of the capsule, the cap is fitted over the body and the body is pushed up until it locks on the cap. The cap can be close fitting and can be approximately half the length of the body, so the cap can travel for a considerable distance down the capsule body before locking. This may have the effect of a piston in trapping and pressurizing the capsule. The excess gas can escape through the gap between the cap and the body, and vents may be provided in this region so as to facilitate the escape of excess pressure. Alternatively, the capsule may utilize a particularly tight locking mechanism rather than vents.

In an embodiment, the capsule is banded by applying a band of polymer solution around the junction between the cap and body. The polymer solution can be a solution of the same polymer as the capsule cap and/or body in a solvent therefor. Banding can provide a smooth capsule surface for coating, which may prevent movement between the cap and body of the capsule.

When preparing the filled capsule that is filled with the composition comprising an avermectin, it is preferred that the composition is in a liquid form at least during the encapsulation process. In an embodiment, the final capsule contains the composition in the liquid form. In an embodiment, the final capsule contains the composition is semi-solid form at room temperature.

### Methods of Administration

The administration of the pharmaceutical composition comprising the avermectin can be carried out via oral, nasal, intraocular, intravenous, intramuscular, subcutaneous, transdermal, subdermal, sublingual or rectal route of administration.

In embodiments, the route of administration is oral and the pharmaceutical composition is provided in the form of capsules, such as soft elastic or hard gelatin capsules.

In an embodiment, the route of administration is nasal. The composition can be a solution, an aerosol, a liquid suspension, or a liquid dispersion, in the form of a nasal spray, a nasal douche, an inhaler, a nasal drop, and/or a diffuser.

In embodiments, the route of administration is dermal including but not limited to topical, subcutaneous, subdermal, transdermal, intradermal or dermal patch.

### Methods of Treatment and Prevention

Provided herein are methods of treating or preventing inflammatory or other disorders by administering to a subject in need thereof a therapeutically or a prophylactically effective amount of a composition disclosed herein.

A “therapeutically effective amount” of a composition comprising (i) a compound of any one of Formulas I-VII, (ii) one or more of a compound of any one of Formulas I-VII, or (iii) ivermectin comprising a compound of Formula VI and a compound of Formula VII is an amount sufficient to confer a therapeutic benefit in a patient after administration, for example to improve in the subject one or more symptoms of the disease. The “therapeutically effective amount” may result in a desired beneficial change of physiology in the subject or to cause an improvement in a clinically significant condition in the subject, for example, by delaying, reducing, minimizing or mitigating one or more symptoms associated with the disease or disorder. Generally, a therapeutically effective amount is also one in which any toxic or detrimental effects of a composition are outweighed by the therapeutically beneficial effects. The effective amount may vary depending on the species, age, weight, sex, health of the subject and the nature or severity of the disease. Depending on the mode of administration, the effective amount may vary as well. In some cases, multiple doses of the composition are administered to achieve the effective amount for the therapeutic benefit intended. In some cases, the therapeutic amount may be used for treating refractory or resistant disorders, and in combination therapies. For example, the effective amount may be administered simultaneously, sequentially, and in the same or different dosage form as an adjunct therapy.

As used herein, the terms “treating”, “treat”, “treatment” refer to reducing, relieving, ameliorating, or alleviating at least one of the symptoms of the disease or disorder. The term includes, for example, administering a formulation as provided herein prevent the onset of inflammation, to reduce or alleviate its severity, and/or to prevent its reoccurrence.

As used herein, the terms “prevent”, “prevention”, and the like refer to acting prior to overt disease or disorder onset, to prevent the disease or disorder from developing or to minimize the extent of the disease or disorder or slow its course of development.

A “prophylactically effective amount” of a composition comprising an (i) a compound of any one of Formulas I-VII, (ii) one or more of a compound of any one of Formulas I-VII, or (iii) ivermectin comprising a compound of Formula VI and a compound of Formula VII refers to an amount of a composition required to achieve a desired prophylactic result. In an embodiment, the prophylactically effective amount is less than the therapeutically effective amount, as a prophylactic dose is used in subjects prior to or at an earlier stage of disease.

A subject may be a mammal, including, but not limited to, a human or non- human mammal. The mammal may be a commercially farmed animal (such as a horse, a cow, a sheep or a pig), a laboratory animal (such as a mouse or a rat), or a pet (such as a cat, a dog, a rabbit or a guinea pig). The subject is preferably a human. The subject may be male or female. Individuals and patients are also subjects herein.

### Inflammatory Disorders

The disclosure provides a method of treating or preventing an inflammatory disorder by administering to a subject in need thereof a therapeutically or a prophylactically effective amount of (i) a compound of any one of Formulas I-VII, (ii) one or more of a compound of any one of Formulas I-VII, or (iii) ivermectin comprising a compound of Formula VI and a compound of Formula VII.

Ivermectin can be used as an anti-inflammatory agent. The anti-inflammatory effect of ivermectin can be achieved via its reduction of pro-inflammatory cytokine production.

Inflammatory Disorders Associated with the Autoimmune Response

Th17 cells are subset of helper CD4⁺ T cells that secrete pro-inflammatory cytokines interleukin (IL)-17 and IL-21. The pro-inflammatory activity of Th17 cells can be beneficial to the host during infection, but uncontrolled Th17 function has been linked and actively involved in several autoimmune pathologies, such as limbic encephalitis, rheumatoid arthritis (RA), multiple sclerosis (MS), systemic lupus erythematosus (SLE), psoriasis, Crohn's disease and development of graft-versus-host disease (GVHD). For example, it has been shown that Th17 cells coordinate joint inflammation and mediate bone destruction in autoimmune arthritis with IL-17 being upregulated in joint tissues in the early phase of RA. Additionally, in experimental autoimmune encephalomyelitis (EAE), the preclinical model of MS, brain autoimmunity is driven by a proinflammatory T cell response that promotes infiltration of immune cells into the central nervous system (CNS), Th17 cells representing the first wave of pathogenic T cells infiltrating the CNS. The development and function of Th17 cells depends on activation of specific intracellular signaling pathways. The steroid receptor-type nuclear receptor RORγt is selectively expressed in Th17 cells and appears to be required for IL-17 production. The induction of RORγt is mediated by IL-6, IL-21, and IL-23 via a STAT3-dependent mechanism.

In addition to T cells, B cells and innate immune cells also secrete pro-inflammatory cytokines in response to tissue stress, injury, and infection via Toll-like receptor (TLRs) signaling activation, which plays a major role in acute inflammatory response. Activation of TLR4 by lipopolysaccharides (LPS) initiates a signaling cascade leading to robust secretion of tumor necrosis factor (TNF), IL-1, and TL-6 through activation of nuclear factor kappa B (NF-kB) transcription factor. These cytokines are pleiotropic proteins that regulate the cell death of inflammatory tissues, modify vascular endothelial permeability, recruit blood cells to inflamed tissues, and induce the production of acute-phase proteins. In addition, both IL-1 and TL-6 play central role in Th17 differentiation and stimulation of IL-17 secretion in T cells demonstrating a dynamic crosstalk between pro-inflammatory adaptive and innate arms of the immune response.

The disclosure provides a method of treating, preventing and/or reducing the severity or extent of inflammatory disorders associated with the autoimmune response by administering to a subject in need thereof a or a prophylactically effective amount of (i) a compound of any one of Formulas I-VII, (ii) one or more of a compound of any one of Formulas I-VII, or (iii) ivermectin comprising a compound of Formula VI and a compound of Formula VII.

The disclosure provides a method for treating, preventing and/or reducing the severity or extent of inflammatory disorders associated with the autoimmune response associated with a disease including but not limited to limbic encephalitis, rheumatoid arthritis (RA), systemic lupus erythematosus (SLE), psoriasis, Crohn's disease and development of graft-versus-host disease (GVHD).

The disclosure provides a method for treating, preventing and/or reducing the severity or extent of multiple sclerosis (MS). Most patients experience a relapsing-remitting (RRMS) course at the initial stage, characterized by unpredictable relapses followed by periods of partial or complete recovery (remission). This phase is characterized by inflammation. At some point, MS becomes progressive (secondary progressive MS (SPMS), characterized by neurodegeneration). In embodiments, provided is a method for treating, preventing and/or reducing the severity or extent of SPMS. In embodiments, provided is a method for treating, preventing and/or reducing the severity or extent of RRMS.

Inflammatory Disorders Associated with Neuroinflammation

Recent studies have demonstrated that the pro-inflammatory cytokines, including IL-17, TNF-α, IL-6, etc., are key players in driving neuroinflammation and have been involved in pathogenesis of plethora of neurological diseases including Alzheimer's disease (AD), Parkinson's disease (PD), stroke, seizure disorders, epilepsy, brain injury, and amyotrophic lateral sclerosis (ALS), etc. TLRs are present in the CNS, specifically expressed on microglia, neurons, astrocytes. It has been shown that TLR4 activation by LPS drives pro-inflammatory cytokine secretion from microglia that subsequently suppresses gamma-aminobutyric acid (GABA) receptor activities at the postsynaptic site and results in physiological as well as behavior changes such as retardation of motor activity. Accordingly, provided herein are methods of targeting pro-inflammatory cytokines as a valuable therapeutic approach for treatment of a broad range of both inflammatory and neurological disorders.

The disclosure provides a method of treating, preventing and/or reducing the severity or extent of inflammatory disorders associated with neuroinflammation by administering to a subject in need thereof a or a prophylactically effective amount of (i) a compound of any one of Formulas I-VII, (ii) one or more of a compound of any one of Formulas I-VII, or (iii) ivermectin comprising a compound of Formula VI and a compound of Formula VII.

The disclosure provides a method for treating, preventing and/or reducing the severity or extent of inflammatory disorders such as several neuroinflammation associated pathologies including but not limited to Alzheimer's disease (AD), Parkinson's disease (PD), stroke, seizure disorders, epilepsy, , and amyotrophic lateral sclerosis (ALS), by administering to a subject in need thereof a therapeutically effective amount of a composition, or compositions, comprising one or more avermectins, and particularly ivermectin or an ivermectin derivative. The disclosure provides a method for treating, preventing and/or reducing the severity or extent of neuroinflammation associated with brain injury, In one embodiment, the brain injury is traumatic brain injury. In embodiments, the inflammatory disorder is epilepsy. In embodiments, provided is a method of reducing seizures in a subject with epilepsy comprising administering to a subject in need thereof a therapeutically effective or a prophylactically effective amount of a pharmaceutical composition disclosed herein. In embodiments, the seizures are focal seizures. In embodiments, the seizures are generalized tonic-clonic seizures. In embodiments, provided is a method of increasing the quality of life in a subject with epilepsy comprising administering to a subject in need thereof a therapeutically effective or a prophylactically effective amount of a pharmaceutical composition disclosed herein. Provided herein is a method of reducing plasma levels of inflammatory cytokines in a subject with epilepsy comprising administering to a subject in need thereof a therapeutically effective or a prophylactically effective amount of a pharmaceutical composition disclosed herein. In embodiments, the inflammatory cytokines are one or more of IL-1β, IL-6, IL-10, IL-12p70, IL-17, IL-21, IL-23, IFN-α, IFN-γ, TNF-α, and CXCL13.

Provided herein is a method of treating, preventing and/or reducing the severity or extent of infantile spasms. The onset of infantile spasms occurs usually between 4 and 7 months. Patients experience hundreds of seizures per day, with the disease leading to developmental regression. The disease has neuroinflammatory cause and children with infantile spasms have been shown to exhibit a degree of inflammation.

Certain inflammatory disorders have been associated with pain, neurological disorders, and spasticity. Recent studies by the inventors have shown that ivermectin can be useful for the treatment of pain, neurological disorders, and spasticity (see U.S. Provisional Application entitled “Methods of Using Avermectin Compositions for the Treatment of Pain and Dosing Regimens”; U.S. Provisional Application, entitled “Methods of Using Avermectin Compositions for the Treatment of Neurological Disorders and Dosing Regimens”; and U.S. Provisional Application entitled “Methods of Using Avermectin Compositions for the Treatment of Spasticity and Dosing Regimens”, all filed on filed Mar. 22, 2022 by the same Applicant, all of which are incorporated herein in their entirety). Accordingly, the disclosure provides a method to treating or preventing pain, neurological disorders, and/or spasticity associated with inflammation.

### Methods of Assessing Efficacy

Provided herein are methods of determining whether a patient is responsive to treatment with a composition disclosed herein. Provided herein is a method of assessing the efficacy of treating an inflammatory disorder disclosed herein with a composition disclosed herein, the method comprising: (a) obtaining a sample from a patient that has received treatment with a composition disclosed herein; (b) measuring the level of one or more inflammatory cytokines in the sample; (c) if the level of the one or more inflammatory cytokines in the sample subceeds (i.e., is lower than) a control level for the one or more inflammatory cytokines, determining that the patient is responsive to the treatment. In some embodiments, the one or more inflammatory cytokines are selected from interleukin (IL)-1β, IL-6, IL-10, IL-12p70, IL-17, IL-21, IL-23, interferon (IFN)-α, IFN-γ, TNF-α, and C-X-C Motif Chemokine Ligand (CXCL)13.

In some embodiments, the inflammatory disorder is an autoimmune disorder. In some embodiments, the autoimmune disorder is limbic encephalitis, rheumatoid arthritis (RA), systemic lupus erythematosus (SLE), Psoriasis, Crohn's disease or graft-versus-host disease (GVHD). In some embodiments, the inflammatory disorder is a neuroinflammatory disorder. In some embodiments, the neuroinflammatory disorder is Alzheimer's disease (AD), Parkinson's disease (PD), stroke, seizure disorders, epilepsy, infantile spasms, or amyotrophic lateral sclerosis (ALS).

In some embodiments, the control level for the one or more inflammatory cytokines is the level that is present in an individual or a population of healthy individuals having the inflammatory disorder that the patient is treated for. In some embodiments, the control level for the one or more inflammatory cytokines is the level for the one or more inflammatory cytokines obtained from the same patient at a different point in time. In some embodiments, the control level for the one or more inflammatory cytokines is the level for the one or more inflammatory cytokines obtained from the same patient before the start of treatment. Provided herein is a method of assessing the efficacy of treating an inflammatory disorder disclosed herein with a composition disclosed herein, the method comprising: (a) obtaining a sample from a patient that has received treatment with a composition disclosed herein; (b) measuring the level of one or more inflammatory cytokines in the sample; (c) if the level of the one or more inflammatory cytokines in the sample subceeds (i.e., is lower than) a control level for the one or more inflammatory cytokines, determining that the patient is responsive to the treatment, wherein the control level for the one or more inflammatory cytokines was determined for the same patient before start of the treatment.

In embodiments, the person skilled in the art may make an assessment as to the relative degree of efficacy of two treatments, wherein the treatment that leads to a larger reduction in the levels of the one or more inflammatory cytokines as compared to a control is considered to be the more effective treatment.

Methods of measuring the levels of cytokines both on the nucleic acid level and the protein level are known in the art. For example, cytokines can be detected using, for example, cytokine bioassays, immunoassays, microfluidic platforms, and enzyme-linked immunosorbent assays (ELISA). The messenger RNA (mRNA) levels of cytokines can be determined, for example, using reverse transcription polymerase chain reaction (RT-PCR).

### Dosing Regimens

Provided herein are methods of treating or preventing an inflammatory disorder including, but not limited to an autoimmune disorder or a neuroinflammatory disorders, by administering to a subject in need thereof a therapeutically or a prophylactically effective amount of a composition, the method comprising administering one of the compositions disclosed herein at a dose of about 10 mg to about 120 mg of (i) a compound of any one of Formulas I-VII, (ii) one or more of a compound of any one of Formulas I-VII, or (iii) ivermectin comprising a compound of Formula VI and a compound of Formula VII. In some embodiments, the method of treatment or prevention includes administering a composition disclosed herein at a dose of about 10 mg to about 80 mg of (i) a compound of any one of Formulas I-VII, (ii) one or more of a compound of any one of Formulas I-VII, or (iii) ivermectin comprising a compound of Formula VI and a compound of Formula VII. In some embodiments, the method of treatment or prevention includes administering a composition disclosed herein at a dose of about 10 mg to about 60 mg of (i) a compound of any one of Formulas I-VII, (ii) one or more of a compound of any one of Formulas I-VII, or (iii) ivermectin comprising a compound of Formula VI and a compound of Formula VII. In some embodiments, the method of treatment or prevention includes administering a composition disclosed herein at a dose of about 20 mg to about 40 mg of (i) a compound of any one of Formulas I-VII, (ii) one or more of a compound of any one of Formulas I-VII, or (iii) ivermectin comprising a compound of Formula VI and a compound of Formula VII. In some embodiments, the method of treatment or prevention includes administering a composition disclosed herein at a dose of about 1 mg, about 3 mg, about 5 mg, about 10 mg, about 20 mg, about 40 mg, about 60 mg, about 80 mg, or about 120 mg of (i) a compound of any one of Formulas I-VII, (ii) one or more of a compound of any one of Formulas I-VII, or (iii) ivermectin comprising a compound of Formula VI and a compound of Formula VII. In preferred embodiments, the composition comprises ivermectin comprising a compound of Formula VI and a compound of Formula VII. In embodiments, a dose of about 10 mg to about 120 mg of the compound is administered to the subject. In embodiments, a dose of about 10 mg to about 80 mg of the compound is administered to the subject. In embodiments, a dose of about 20 mg to about 40 mg of the compound is administered to the subject. In embodiments, a dose of about 10 mg to about 30 mg of the compound is administered to the subject. In embodiments, a dose of about 15 mg to about 25 mg of the compound is administered to the subject. In embodiments, a dose of about 50 mg to about 70 mg of the compound is administered to the subject. In embodiments, a dose of about 55 mg to about 65 mg of the compound is administered to the subject. In embodiments, about 10 mg, about 20 mg, about 30 mg, about 40 mg, about 50 mg, about 60 mg, about 70 mg, about 80 mg, about 90 mg, about 100 mg, about 110 mg, or about 120 mg of the compound is administered to the subject. The dose may be daily.

The dosage regimens for the therapy may be adjusted to provide the optimum desired response (e.g., a therapeutic or prophylactic response). For example, a single dose may be administered, several divided doses may be administered over time or the dose may be proportionally reduced or increased depending on the subject's responsiveness to the therapy. In some embodiments, the composition is administered twice per day (BD), once per day (QD), once every other day, once every three days. In some embodiments, the dosage schedule is once a day, twice a day, every other day or once every three days for about 14 days, for about 30 days, for about 60 days, for about 84 days, for about 90 days, or continuously. In some embodiments, the dosage schedule is daily, every other day, 2-4 times a week, 3-5 times a week, weekly, biweekly, monthly, or bimonthly for about 90 days, for about 6 months, for about 1 year or continuously. In an embodiment, the dosage schedule is once a day for about 14 days. In an embodiment, the dosage schedule is once a day for about 84 days.

In an embodiment, the pharmaceutical composition comprises: (i) about 8% ivermectin; (ii) about 27.6% mono- and di- C₈ to C₁₀ fatty acid esters of glycerol; (iii) about 32.2% polysorbate 80; and (iv) about 32.2% vitamin E TPGS.

In an embodiment, the pharmaceutical composition comprises: (i) about 8% ivermectin; (ii) about 27.6% Masester E8120; (iii) about 32.2% polysorbate 80; and (iv) about 32.2% vitamin E TPGS.

In an embodiment, the pharmaceutical composition comprises: (i) about 8% ivermectin; (ii) about 27.6% mono- and di-C₈ to C₁₀ fatty acid esters of glycerol; (iii) about 18.4% polysorbate 80; and (iv) about 46% vitamin E TPGS.

In an embodiment, the pharmaceutical composition comprises: (i) about 8% ivermectin; (ii) about 27.6% Masester E8120; (iii) about 18.4% polysorbate 80; and (iv) about 46% vitamin E TPGS.

In an embodiment, the pharmaceutical composition comprises: (i) about 8% ivermectin; (ii) about 27.6% mono- and di- C₈ to C₁₀ fatty acid esters of glycerol; and (iii) about 64.4% polysorbate 80.

In an embodiment, the pharmaceutical composition comprises: (i) about 8% ivermectin; (ii) about 27.6% Masester E8120; and (iii) about 64.4% polysorbate 80.

In embodiments, the ivermectin comprises a compound of Formula VI (22,23-dihydroavermectin B1a) and a compound of Formula VII (22,23-dihydroavermectin B1b). In some embodiments, ivermectin comprises at least about 70% of 22,23-dihydroavermectin B1a and less than about 30% of 22,23-dihydroavermectin Bib. In some embodiments, ivermectin comprises at least about 90% of 22,23-dihydroavermectin B1a and less than about 10% of 22,23-dihydroavermectin B1b.

In an embodiment, a pharmaceutical composition comprising (i) about 8% ivermectin; (ii) about 27.6% Masester E8120; (iii) about 32.2% polysorbate 80; and (iv) about 32.2% vitamin E TPGS is administered daily and continuously at a dose of about 10 mg to 120 mg. In an embodiment, a pharmaceutical composition comprising (i) about 8% ivermectin; (ii) about 27.6% Masester E8120; (iii) about 32.2% polysorbate 80; and (iv) about 32.2% vitamin E TPGS is administered daily and continuously at a dose of about 10 mg to 80 mg. In an embodiment, a pharmaceutical composition comprising (i) about 8% ivermectin; (ii) about 27.6% Masester E8120; (iii) about 32.2% polysorbate 80; and (iv) about 32.2% vitamin E TPGS is administered daily and continuously at a dose of about 10 mg to 60 mg. In an embodiment, a pharmaceutical composition comprising (i) about 8% ivermectin; (ii) about 27.6% Masester E8120; (iii) about 32.2% polysorbate 80; and (iv) about 32.2% vitamin E TPGS is administered daily and continuously at a dose of about 20 mg to 40 mg.

In an embodiment, a pharmaceutical composition comprising (i) about 8% ivermectin; (ii) about 27.6% Masester E8120; (iii) about 32.2% polysorbate 80; and (iv) about 32.2% vitamin E TPGS is administered daily. In an embodiment, a pharmaceutical composition comprising (i) about 8% ivermectin; (ii) about 27.6% Masester E8120; (iii) about 32.2% polysorbate 80; and (iv) about 32.2% vitamin E TPGS is administered daily and continuously. In an embodiment, a pharmaceutical composition comprising (i) about 8% ivermectin; (ii) about 27.6% Masester E8120; (iii) about 32.2% polysorbate 80; and (iv) about 32.2% vitamin E TPGS is administered at a dose of about 10 mg daily and continuously. In an embodiment, a pharmaceutical composition comprising (i) about 8% ivermectin; (ii) about 27.6% Masester E8120; (iii) about 32.2% polysorbate 80; and (iv) about 32.2% vitamin E TPGS is administered at a dose of about 20 mg daily and continuously. In an embodiment, a pharmaceutical composition comprising (i) about 8% ivermectin; (ii) about 27.6% Masester E8120; (iii) about 32.2% polysorbate 80; and (iv) about 32.2% vitamin E TPGS is administered at a dose of about 40 mg daily and continuously. In an embodiment, a pharmaceutical composition comprising (i) about 8% ivermectin; (ii) about 27.6% Masester E8120; (iii) about 32.2% polysorbate 80; and (iv) about 32.2% vitamin E TPGS is administered at a dose of about 60 mg daily and continuously. In an embodiment, a pharmaceutical composition comprising (i) about 8% ivermectin; (ii) about 27.6% Masester E8120; (iii) about 32.2% polysorbate 80; and (iv) about 32.2% vitamin E TPGS is administered at a dose of about 80 mg daily and continuously. In an embodiment, a pharmaceutical composition comprising (i) about 8% ivermectin; (ii) about 27.6% Masester E8120; (iii) about 32.2% polysorbate 80; and (iv) about 32.2% vitamin E TPGS is administered at a dose of about 120 mg daily and continuously.

In an embodiment, the inflammatory disorder is an autoimmune disorder. In some embodiments, the autoimmune disorder is limbic encephalitis, rheumatoid arthritis (RA), multiple sclerosis (MS), systemic lupus erythematosus (SLE), psoriasis, Crohn's disease or graft-versus-host disease (GVHD).

In an embodiment, the inflammatory disorder is a neuroinflammatory disorder. In some embodiments, the neuroinflammatory disorder is Alzheimer's disease (AD), Parkinson's disease (PD), stroke, seizure disorders, epilepsy, traumatic brain injury, infantile spams, or amyotrophic lateral sclerosis (ALS).

In some embodiments, the method comprises administering to the subject a pharmaceutical composition disclosed herein, with one or more adjunct therapies, including but not limited to immune suppressive agents such as azathioprine, calcineurin inhibitors, corticosteroids, cyclophosphamide, leflunomide, methotrexate, mycophenolate, and combinations thereof.

### EXAMPLES

### Example 1—Solubility Determination

The active pharmaceutical ingredient (API) used in the following examples, is ivermectin comprising greater than or equal to 90% of 22,23-dihydroavermectin B1a and less than 10% of 22,23-dihydroavermectin B1b. The solubility assessments were performed by continually adding a known amount of the API into each individual excipient and letting the excipient reach solubility equilibrium via API saturation (visible solid observed). The experiments were conducted at ambient conditions (25° C.) and were left to mix on a stir plate at 500 RPM for at least 72 hours. Subsequently, samples were centrifuged and filtered via a 0.45 μm PVDF membrane filter (Millipore Durapore) and filtrate was assayed per United States Pharmacopeia (USP) ivermectin monograph. Quantitative composition of placebo vehicle and the corresponding ivermectin initial solubility results are reported in Table 1. Due to the melting point of Vitamin E TPGS (VE TPGS) at 38° C., the solubility study in VE TPGS was conducted at 40° C. on a hot plate following same procedure as above.

TABLE 1
Ivermectin Solubility Determination at 25° C.

| | Solubility | |
|---|---|---|
| Excipient | (mg/g) | Rank |
| Maisine CC | 60.17 | 10 |
| Capryol 90 | 150.92 | 2 |
| LauroglycolTM FCC | 71.96 | 9 |
| PEG 400 | 112.92 | 4 |
| Masester E8120 | 164.06 | 1 |
| Labrafil M 2125 CS | 35.32 | 14 |
| Tween 80 | 88.26 | 7 |
| Labrasol ALF | 126.36 | 3 |
| Kolliphor EL | 95.85 | 6 |
| Vitamin E TPGS | 82.98 | 8 |
| Soybean oil | 6.72 | 18 |
| Olive oil | 4.71 | 19 |
| Fractionated Coconut oil | 16.55 | 16 |
| Triacetin | 58.75 | 11 |
| Oleic acid | 48.58 | 13 |
| Medium Chain Triglyceride | 16.49 | 17 |
| Span 20 | 97.66 | 5 |
| Span 80 | 54.04 | 12 |
| Glycerin | <0.1 | 20 |
| Propylene glycol | 25.61 | 15 |

### Example 2—Excipient Compatibility Study

An excipient compatibility study was initiated with selected excipients ranging from oils to surfactants based on equilibrium solubility results (Table 2). The study was designed to provide an ivermectin solution at approximately 5% in selected excipients as in Table 2 with the exception of excipients that the equilibrium solubility are below 5%, namely fractionated coconut oil and oleic acid at approximately 1%. After solutions were prepared using a hand-held homogenizer (Model IKAT10 Ultra-Turrax), each excipient sample was aliquoted in amber vials, approximately 10% water was added in each excipient aliquot, and bench scale gel stripe OET-004037 was also made and added in a separate set of aliquot samples at approximately 1:1 w/w.

All samples were stored at 40° C./75% relative humidity (RH) condition (Model: Lunaire environmental chamber) and tested by developmental reversed phase HPLC method for Assay/Related Substances at T=0, 2 weeks, and 1 month. Results are summarized in Table 3 and Table 4.

Oily vehicles including Maisine CC, fractionated coconut oil, and oleic acid had good chemical compatibility with the API, among which Maisine CC has the highest equilibrium solubility, therefore Maisine CC was chosen to be studied further as an oily vehicle. Tween 80, Labrasol ALF and Vitamin E TPGS categorized as surfactant were compared and Vitamin E TPGS showed the best chemical compatibility with the API with minimum increase in impurities over time, followed by Tween 80 having less stability over time. Both Vitamin E TPGS and Tween 80 were selected as emulsifiers to be used in formulation development. Due to the high solubility in Masester E8120, Masester E8120 was selected for further evaluation for formulation development as solubilizer and emulsifer. PEG 400, as a co-solvent was not considered for further formulation development, due to its poor chemical compatibility.

TABLE 2
Excipients list for Compatibility Study and Actual API w/w %

| | |
|---|---|
| | % w/w |
| Excipient Name | (Corrected for API purity) |

Oily Vehicle

| | |
|---|---|
| Maisine CC | 4.54% |
| Fractionated coconut oil | 0.91% |
| Oleic acid | 0.95% |

Surfactant

| | |
|---|---|
| Tween 80 | 4.62% |
| Labrasol ALF | 4.70% |
| Vitamin E TPGS | 4.61% |

Mixed Glycerides

| | |
|---|---|
| Masester E8120 | 4.63% |

Co-solvent

| | |
|---|---|
| PEG 400 | 4.61% |

TABLE 3
Summary of Excipient Compatibility Study (Assay)

| | Chromatographic purity by HPLC (% area) | | |
|---|---|---|---|
| Excipients | T = 0 | T = 2 weeks | T = 1 months |

Sample: Control

| | | | |
|---|---|---|---|
| Maisine CC | 97.13 | 96.00 | 96.82 |
| Tween 80 | 97.53 | 91.87 | 88.45 |
| Labrasol ALF | 97.74 | 89.16 | 81.35 |
| Vitamin E TPGS | 97.74 | 97.43 | 97.37 |
| Fractionated coconut oil | 95.61 | 97.94 | N/A |
| Oleic acid | 99.60 | 83.55 | N/A |
| Masester E8120 | 97.58 | 96.81 | 97.06 |
| PEG 400 | 97.58 | 89.36 | N/A |

Sample: 10% water

| | | | |
|---|---|---|---|
| Maisine CC | N/A | 95.90 | 96.83 |
| Tween 80 | N/A | 93.02 | 91.18 |
| Labrasol ALF | N/A | 93.91 | 89.91 |
| Vitamin E TPGS | N/A | 96.99 | 97.46 |
| Fractionated coconut oil | N/A | 97.83 | N/A |
| Oleic acid | N/A | 86.02 | N/A |
| Masester E8120 | N/A | 95.11 | 91.68 |
| PEG 400 | N/A | 94.35 | N/A |

Sample: Gel Stripe (1:1 w/w)

| | | | |
|---|---|---|---|
| Maisine CC | N/A | 96.15 | 96.94 |
| Tween 80 | N/A | 93.97 | 93.51 |
| Labrasol ALF | N/A | 97.11 | 96.49 |
| Vitamin E TPGS | N/A | 97.50 | 97.74 |
| Fractionated coconut oil | N/A | 94.42 | N/A |
| Oleic acid | N/A | 83.73 | N/A |
| Masester E8120 | N/A | 97.35 | 97.31 |
| PEG 400 | N/A | 96.92 | N/A |

TABLE 4
Results Summary for Compatibility Study
of Excipient Samples (Total impurities).
% adjusted area = Total Impurities %
excluding API impurities %

| | Total Impurities % (% adjusted area) | | |
|---|---|---|---|
| Excipients | T = 0 | T = 2 weeks | T = 1 months |

Sample: Control

| | | | |
|---|---|---|---|
| Maisine CC | 0.58 | 0.35 | 0.71 |
| Tween 80 | 0.16 | 4.86 | 10.55 |
| Labrasol ALF | <LOQ | 8.67 | 20.49 |
| Vitamin E TPGS | <LOQ | <LOQ | 0.11 |
| Fractionated coconut oil | <LOQ | <LOQ | N/A |
| Oleic acid | <LOQ | 17.27 | N/A |
| Masester E8120 | 0.17 | 0.58 | 0.45 |
| PEG 400 | 0.10 | 8.54 | N/A |

Sample: 10% water

| | | | |
|---|---|---|---|
| Maisine CC | N/A | 0.45 | 0.69 |
| Tween 80 | N/A | 3.53 | 6.94 |
| Labrasol ALF | N/A | 3.46 | 8.76 |
| Vitamin E TPGS | N/A | 0.42 | <LOQ |
| Fractionated coconut oil | N/A | <LOQ | N/A |
| Oleic acid | N/A | 9.89 | N/A |
| Masester E8120 | N/A | 2.27 | 6.60 |
| PEG 400 | N/A | 3.15 | N/A |

Sample: Gel Stripe (1:1 w/w)

| | | | |
|---|---|---|---|
| Maisine CC | N/A | 0.33 | 0.58 |
| Tween 80 | N/A | 2.43 | 4.22 |
| Labrasol ALF | N/A | 0.30 | 1.06 |
| Vitamin E TPGS | N/A | <LOQ | <LOQ |
| Fractionated coconut oil | N/A | 3.28 | N/A |
| Oleic acid | N/A | 11.82 | N/A |
| Masester E8120 | N/A | 0.11 | 0.18 |
| PEG 400 | N/A | 0.50 | N/A |

### Example 3—Fiber Optic Dispersion Study

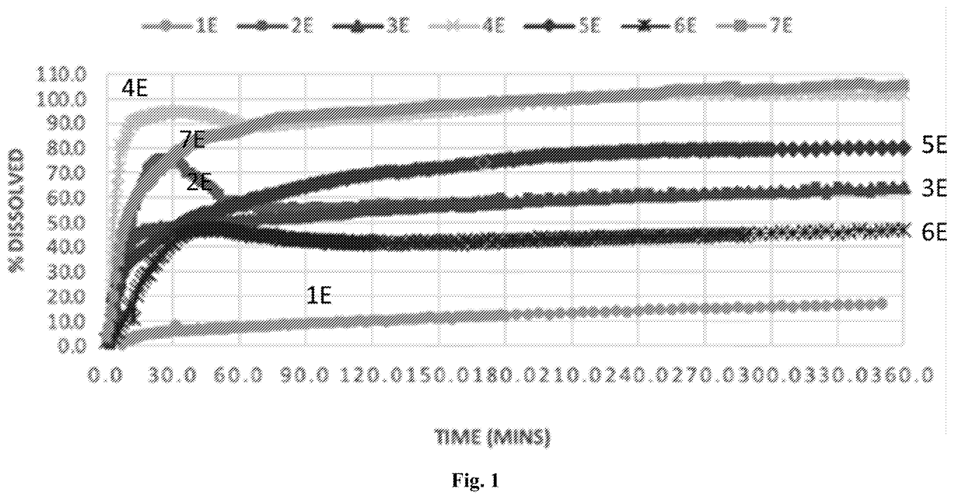

Fiberoptic Dispersion in fasted state simulated intestinal fluid (FaSSIF) and FaSSIF-V2 were performed using a Distec dissolution system 2500 and PIONfiber optic dissolution system to collect real-time dissolution data and data processing. With a target of 20 mg/capsule and based on the compatibility studies, a range of formulations (Table 5) were designed for dispersibility evaluations. FaSSIF medium was chosen to mimic intestinal environment after ivermectin is ingested orally. Dispersion analysis was conducted firstly in 500 mL Fasted State Simulated Intestinal Fluid (pH˜6.5) at 50 rpm paddle speed (USP <711> Apparatus II) for screening various excipient combinations and compositions for approximately 6 hours. Results were plotted as a graph shown in FIG. 1.

FaSSIF-V2 was used later to confirm dispersibility of formulations 7E and 9E as they have shown higher dispersibility than other formulation evaluated from the first round of the dispersion study.

Kinetic solubility in FaSSIF and FaSSIF-V2 for both 7E and 9E reached 800% release at T=30 minutes suggesting an immediate release profile. No drug precipitation was observed indicating good dispersibility of both formulations and ability to maintain solubilization of API with low risk of API precipitation once ingested into GI tract. Data processing using Aupro.

TABLE 5
Quantitative Compositions of Formulations in Dispersion Study

| Formulation # | Components | % w/w |
|---|---|---|
| 1E | Ivermectin | 10.0% |
| | Masester E8120 | 90.0% |
| | Total | 100% |
| 2E | Ivermectin | 5.0% |
| | Maisine CC | 47.5% |
| | Tween 80 | 47.5% |
| | Total | 100% |
| 3E | Ivermectin | 10.0% |
| | Masester E8120 | 63.0% |
| | VE TPGS | 27.0% |
| | Total | 100% |
| 4E | Ivermectin | 8.0% |
| | Masester E8120 | 27.6% |
| | Tween 80 | 64.4% |
| | Total | 100% |
| 5E | Ivermectin | 10.0% |
| | Masester E8120 | 45.0% |
| | Tween 80 | 18.0% |
| | VE TPGS | 27.0% |
| | Total | 100% |
| 6E | Ivermectin | 5.0% |
| | Maisine CC | 47.5% |
| | Tween 80 | 19.0% |
| | VE TPGS | 28.5% |
| | Total | 100% |
| 7E | Ivermectin | 8.0% |
| | Masester E8120 | 27.6% |
| | Tween 80 | 32.2% |
| | VE TPGS | 32.2% |
| | Total | 100% |
| 9E | Ivermectin | 8.0% |
| | Masester E8120 | 27.6% |
| | Tween 80 | 18.4% |
| | VE TPGS | 46.0% |
| | Total | 100% |

### Example 4—Analysis of Prototype Formulations

7E and 9E were selected as prototype formulations following the kinetic solubility study where both formulations showed good dispersibility in simulated intestinal fluids. A 12-month informal stability study was conducted to challenge these two formulations at 40° C./75% RH in a Lunaire environmental chamber and with the addition of 5% water and gel stripes (OET-004037) in separate vials to mimic softgel water migration and the impact of gel on formulation in softgel dosage form, respectively. The formulation control samples (without water and gel stripes) were stored in 8 oz amber Boston round bottles to mimic preliminary intended packaging for first in human clinical study (SAD/MAD study). All other samples were stored in amber glass vials. The Assay/RS results are summarized in Tables 6 and 7, analyzed by a developmental HPLC method.

There was no precipitation nor phase separation observed at T=0 to 1 month at 40° C./75% RH in all sample vials. There was light slurry observed in the T=3-month water added sample in 7E which indicates minor API precipitation with 5% water addition possibly due to polymorphism change of API. However, water added samples is a simulation and not a true representation of water migration in softgels. The minor precipitation in this sample is inconclusive and deemed to be minor in terms of formulation stability.

TABLE 6
Results Summary for Informal Stability
Study of Prototype Formulations (Assay)

| Prototype | Chromatographic purity by HPLC (% LC) | | | |
|---|---|---|---|---|
| Formulation | T = 0 | T = 2 weeks | T = 1 months | T = 3 months |

Sample: Control

| | | | | |
|---|---|---|---|---|
| 7E | 99.22 | 100.52 | 101.10 | 94.02 |
| 9E | 99.59 | 98.32 | 98.34 | 98.68 |

Sample: 10% water

| | | | | |
|---|---|---|---|---|
| 7E | N/A | 97.00 | 96.29 | 98.07 |
| 9E | N/A | 94.24 | 92.82 | 101.45 |

Sample: Gel Stripe (1:1 w/w)

| | | | | |
|---|---|---|---|---|
| 7E | N/A | 97.51 | 97.55 | 94.20 |
| 9E | N/A | 96.62 | 96.23 | 104.00 |

TABLE 7
Results Summary for Informal Stability Study of
Prototype Formulations (Related Substances)

| Prototype | Chromatographic purity by HPLC (% area) | | | |
|---|---|---|---|---|
| Formulation | T = 0 | T = 2 weeks | T = 1 months | T = 3 months |

Sample: Control

| | |
|---|---|
| 7E | 6.56 |
| 9E | 6.05 |

Sample: 10% water

| | |
|---|---|
| 7E | 5.67 |
| 9E | 5.33 |

Sample: Gel Stripe (1:1 w/w)

| | |
|---|---|
| 7E | 3.25 |
| 9E | 3.55 |

### Example 5—Freeze-Thaw Study

Prototype formulations 7E and 9E were challenged in a freeze-thaw study for physical and chemical stability from −15° C. to 40° C. in a ChinaCell environmental test chamber for a 7-day period of time, 24-hour cycle. There was no precipitation nor phase separation observed from two samples vials at day 7. Chemical stability (Assay) is summarized in Table 8. It can be concluded that both formulations are stable under extreme temperature fluctuation stress.

TABLE 8
Results Summary for Freeze-thaw Study
of Prototype Formulations (Assay)

Chromatographic purity by HPLC (% area)

| Prototypes | T = 0 | T = day 7 |
|---|---|---|
| 7E | 99.22 | 101.3 |
| 9E | 99.59 | 98.80 |

### Example 6—Pharmacokinetic Profiles of Ivermectin B1a in Sprague Dawley Rats Following IV and Oral Administration

Thirty-six male Sprague Dawley rats were randomly assigned into four groups, with nine male rats in each group. All animals in all groups are fasted overnight prior to the experimental date.

Rats in Group 1 were administered ivermectin API solubilized in DMSO, solutol, and saline (n=9) administered intravenously (IV). Rats in Group 2 were administered 2 mg/kg Stromectol crushed and solubilized in water (n=9) via oral gavage. Stromectol is formulated by the manufacturer as a tablet comprising 3 mg ivermectin comprising at least 90% 22,23-dihydroavermectin B1a and less than 10% 22,23-dihydroavermectin B1b. Rats in Group 3 were administered 2 mg/kg Ivomec in water (n=9) via oral gavage. Ivomec is formulated by the manufacturer as a 1% ivermectin solution. Rats in Group 4 were administered 2 mg/kg prototype formulations 7E (“7E”) in water (n=9) via oral gavage.

Blood samples were collected from 6 of 9 animals per group at 0.5, 1, 2, 3, 4, 6, 8, 24, 72, 120, 168 hours post-dose. The collected plasma samples were analyzed for the concentration of Ivermectin B1a by a validated LC-MS/MS method. Pharmacokinetic parameters were calculated by the concentration data of Ivermectin B1a in plasma samples using non-compartment model of Phoenix WinNonlin 7.0 software.

The remaining 3 of 9 animals per group were sacrificed 4 hours after study product administration. Blood samples were collected via jugular vein or other suitable vein. Brains were removed, rinsed with saline, dried with filter paper and weighed immediately. The brain samples were homogenized and the samples were analyzed for the concentration of Ivermectin B1a by a LC-MS/MS method per FDA's guidelines. The brain samples and plasma sample ratios were calculated for the BBB penetration percentages.

### Results

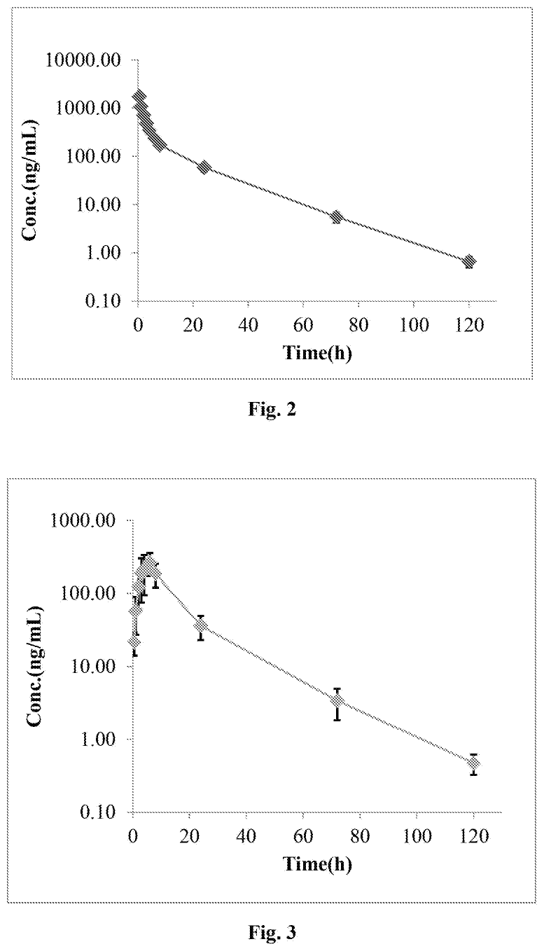

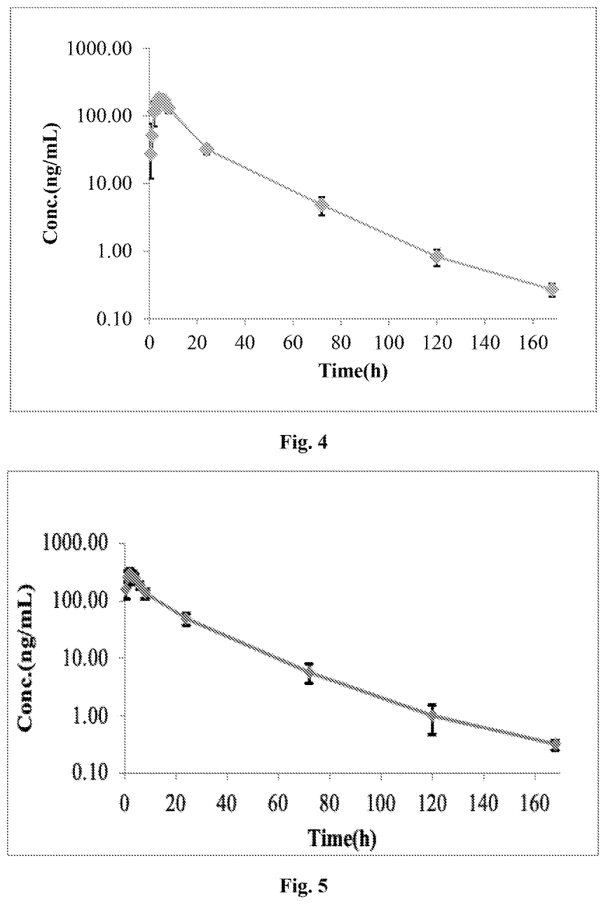

The main pharmacokinetic parameters of Ivermectin B1a in SD rat plasma after single administration of Ivermectin API, Stromectol, Ivomec, and 7E are summarized in Table 9 and FIGS. 2-5.

TABLE 9
Main pharmacokinetic parameters of Ivermectin B1a in SD rat plasma
after single intravenous administration of Ivermectin (API) or
Oral Administration of Stromectol, Ivomec, and 7E (Mean ± SD).

| | Route | | | | | | | |
|---|---|---|---|---|---|---|---|---|
| Group | of | T_{1/2} | Tₘₐₓ | Cₘₐₓ | AUC₍₀₋ₜ₎ | AUC_{(0-∞)} | MRT₍₀₋ₜ₎ | MRT_{(0-∞)} |
| No. | Dosin | h | b | ng/ml | h*ng/ml | h*ng/ml | h | h |
| 1 | IV | 14.82 | 0.50 ± | 1722.67 | 8259.31 ± | 8273.53 ± | 10.58 ± | 10.81 ± |
| API | | | 0.0 | | 1032.9 | 1034.0 | 0.83 | 0.85 |
| 2 | PO | 12.85 | 5.67 ± | 278.4 ± | 4220.94 ± | 4244.6 ± | 13..52 ± | 14.11 ± |
| Stromecto | | | 0.8 | 109.3 | 1594.2 | 1589.3 | 1.49 | 1.35 |
| 3 | PO | 18.49 | 4.83 ± | 190.42 ± | 3396.36 ± | 3410.59 ± | 17.26 ± | 17.85 ± |
| Ivomec | | | 1.3 | 25.36 | 353.94 | 354.24 | 2.75 | 2.63 |
| 4 | PO | 17.15 | 2.17 ± | 306.28 ± | 4677.49 ± | 4691.6 ± | 16.71 ± | 17.15 ± |
| 7E | | | 0.7 | 61.41 | 920.69 | 916.77 | 2.27 | 2.37 |

Dose level: 2 mg/kg.
Fating status: Fasting.
Sex: Male.
n = 6.

Group 1: After a single IV injection of 2 mg/kg Ivermectin API in male SD rats with fasting, the AUC₍₀₋₄₎ of Ivermecin B1a in plasma was 8259.31±1032.95 hr*ng/ml, the T_{1/2} were 14.82±0.72 hr.

Group 2: After a single PO administration of 2 mg/kg Stromectol in male SD rats with fasting, the AUC₍₀₋₄₎ of Ivermecin B1a in plasma was 4220.94±1594.22 hr*ng/ml, the T_{1/2} were 12.85±2.16 hr.

Group 3: After a single PO administration of 2 mg/kg Ivomec in male SD rats with fasting, the AUC₍₀₋₄₎ of Ivermecin B1a in plasma was 3396.36±353.94 hr*ng/ml, the T_{1/2} were 18.49±1.89 hr.

Group 4: After a single PO administration of 2 mg/kg 7E in male SD rats with fasting, the AUC₍₀₋₄₎ of Ivermecin B1a in plasma was 4677.49±920.69 hr*ng/ml, the T_{1/2} were 17.15±13.14 hr.

The ratio between the concentration of Ivermectin B1a in brain and the concentration of Ivermectin B1a in the plasma were calculated and summarized in Table 10.

TABLE 10
Ratio of Ivermectin B1a in SD rat plasma and brain after single
intravenous administration of Ivermectin (API) or oral administration
of Stromectol, Ivomec, and 7E (Mean ± SD).

| | | Conc. of | Conc. of | |
|---|---|---|---|---|
| | Route | ivermectin | ivermectin | Brain:Plasma |
| | of | B1a in brain | B1a in plasma | Ratio |
| Group No. | Dosing | ng/ml | ng/ml | — |
| 1 - API | IV | 79.26 ± 11.97 | 462.58 ± 43.49 | 0.171 ± 0.010 |
| 2 - | PO | 13.97 ± 4.97 | 299.25 ± 71.61 | 0.046 ± 0.006 |
| Stromectol | | | | |
| 3 - Ivomec | PO | 8.08 ± 3.81 | 235.98 ± 63.50 | 0.033 ± 0.008 |
| 4 - 7E | PO | 12.84 ± 0.36 | 301.27 ± 46.06 | 0.043 ± 0.005 |

Dose level: 2 mg/kg. Fating status: Fasting. Sex: Male. n = 3.

Group 1: After a single IV injection of 2 mg/kg Ivermectin API in male SD rats with fasting, the ratio between the concentration of Ivermectin B1a in the brain and the concentration of Ivermectin B1a in the plasma was 0.171±0.010.

Group 2: After a single PO administration of 2 mg/kg Stromectol in male SD rat with fasting, the ratio between the concentration of Ivermectin B1a in the brain and the concentration of Ivermectin B1a in the plasma was 0.046±0.006.

Group 3: After a single PO administration of 2 mg/kg Ivomec in male SD rats with fasting, the ratio between the concentration of Ivermectin B1a in the brain and the concentration of Ivermectin B1a in the plasma was 0.033±0.008.

Group 4: After a single PO administration of 2 mg/kg 7E in male SD rats with fasting, the ratio between the concentration of Ivermectin B1a in the brain and the concentration of Ivermectin B1a in the plasma was 0.043±0.005.

### Conclusion

After a single fasting oral administration of 2 mg/kg Stromectol and 7E there was no significant difference in Cmax (Stromectol vs 7E 1:1.10) or AUC₀₋₄(Stromectol vs 7E 1:1.11).

### Example 7—Inhibition by Ivermectin of IL-17, IL-21, and IFN-γ Secretion in Human Th17-Skewed CD4⁺ T Cells

Inflammatory mediators, such as pro-inflammatory cytokines, play an important role in driving the pathophysiology of seizures in various forms of epilepsy of different etiologies. Pro-inflammatory cytokines including IL-17, IL-21, IFN-γ, IL-1β, TL-6 and TNFα are secreted by different brain cells as well as peripheral immune cells in response to acute seizures or other primary brain injury followed by a cascade of downstream inflammatory events leading to neuronal cell loss, blood brain barrier (BBB) disruption and altered neuronal excitability. These events generate a positive feedback loop to promote chronic brain inflammation that results in secondary damage to the brain and increase the likelihood of recurrent seizures with increased severity.

### Methods

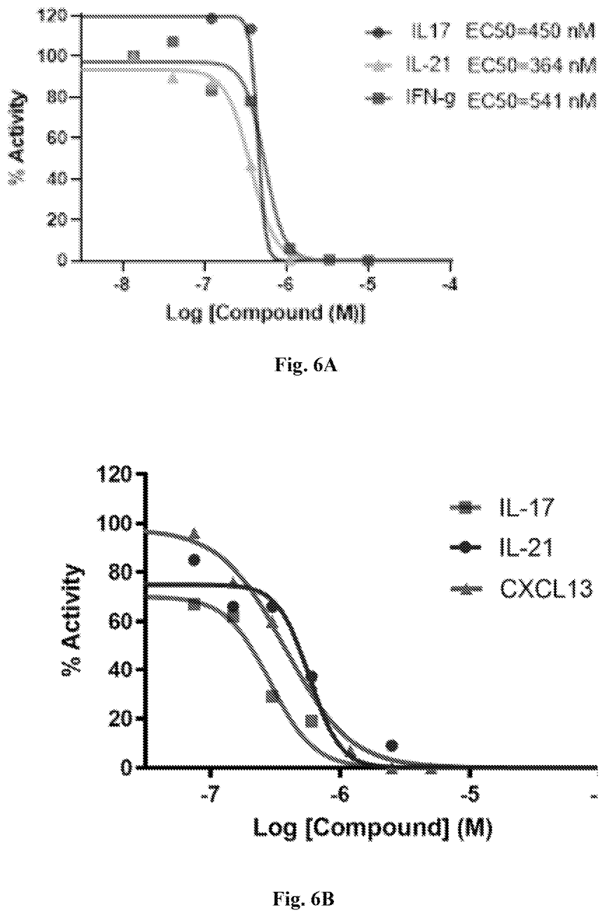

Peripheral blood CD4⁺ T cells were treated with indicated doses of ivermectin and then stimulated by anti-CD3/CD28 mAbs, IL-1β (50 ng/mL) and TGF-β (5 ng/mL) for 48 hours. The supernatants were analyzed for IL-17, TL-21 and IFN-γ by ELISA. One representative of three different experiments is shown in FIG. 6.

### Results

Activation of resting T cells, resulting in cytokine secretion and proliferation, requires two distinct signals from antigen-presenting cells (APCs), mimicked by co-stimulation of the T cell receptor (TCR)/CD3 complex and the CD28 receptor. By using stimulatory antibodies against CD3 and CD28 (anti-CD3/CD28) in combination with IL-1p3 and TGF-β (Th17-skewing conditions), it was found that Ivermectin robustly inhibited IL-17, IL-21, and IFN-γ secretion Th17-skewed human CD4⁺ T cells in a dose-dependent manner with 50% of its maximum effect (EC50) being observed at 450 nM, 364 nM and 541 nM, respectively, as shown in FIG. 6.

### Example 8—Reduction of Th17-Induced STAT3 and IkBα Phosphorylation by Ivermectin in a Dose-Dependent Manner

### Methods

Peripheral blood CD4⁺ T cells were treated with indicated doses of ivermectin and then stimulated by anti-CD3/CD28 mAbs, IL-1β (50 ng/mL) and TGF-0 (5 ng/mL) for 2 hours. The cell lysates were prepared and analyzed by Western Blot. One representative of three different experiments is shown.

### Results

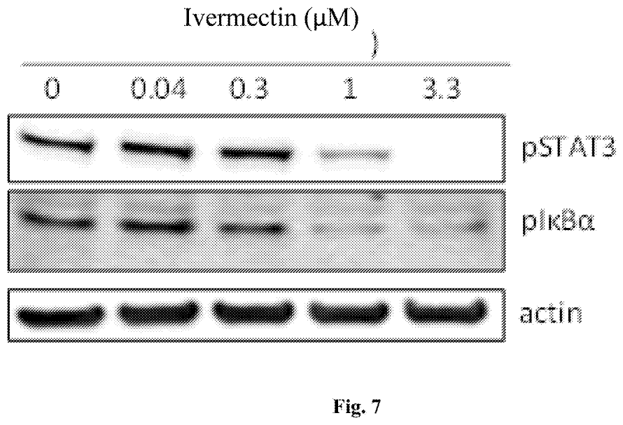

The induction and expression of IL-17 and IL-21 has been shown to be dependent on activation of certain transcription factors including STAT3, RAR-related orphan receptor (ROR) γt and nuclear factor kappa B (NF-κB). STAT3 activation leads to STAT3 phosphorylation and translocation to the nucleus where it directly binds to the IL-17 and IL-21 promoters. Treatment of human CD4⁺ T cells with ivermectin during Th17-skewing conditions led to a dose-dependent reduction of both STAT3 and inhibitor molecule protein (IkB)α phosphorylation (FIG. 7), consistent with the ability of ivermectin to down-regulate pro-inflammatory cytokine secretion under the same stimulatory conditions.

### Example 9—Ivermectin Up-Regulates the Expression of FoxP3 and Increases the Percentage of FoxP3⁺ Regulatory T Cells (Treg)

While Th17 cells drive the inflammation, Foxp3⁺ regulatory T cells (Tregs) play a protective role and prevent unwanted auto-aggressive immune responses by secreting immuno-suppressive cytokines or by direct inhibition of target cell function. The proper balance between Th17 and Treg arms of the immune response is important for timely resolution of both peripheral and local inflammatory responses.

### Methods

Peripheral blood CD4⁺ T cells were treated with 1.5 mM of ivermectin and then stimulated by anti-CD3/28 mAbs, IL-1β (50 ng/mL) and TGF-β (5 ng/mL) for 5 days. FoxP3, IL-17A and RORc mRNA expression levels were tested by RT-PCR (A). The percentage of FoxP3⁺ T cells was determined by Flow Cytometry by using intra-cellular staining with PE-conjugated anti-Foxp3 antibody (B).

### Results

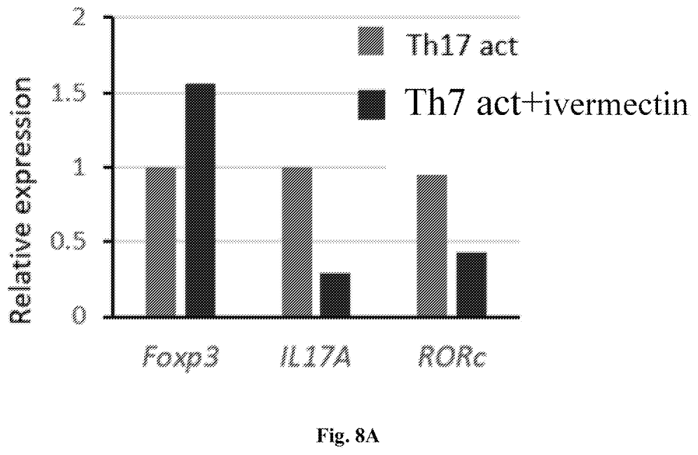

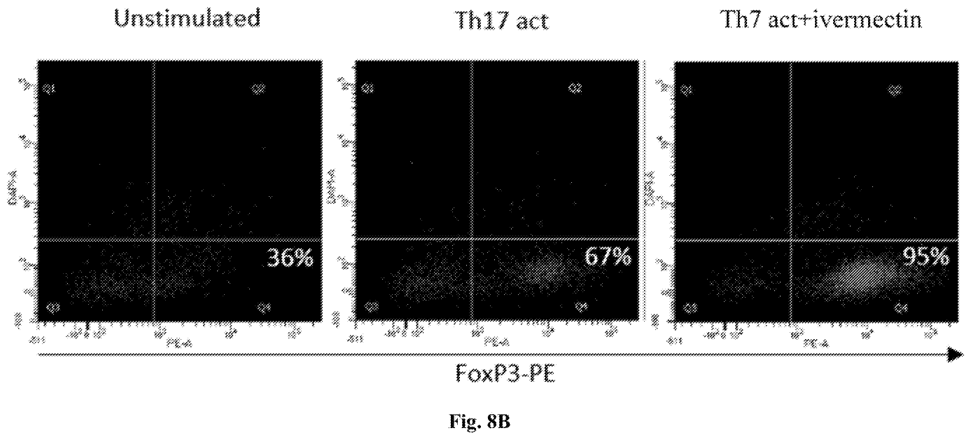

While Th17 cells are actively involved in pathogenesis of majority of pro-inflammatory disorders, Tregs play a protective role and prevent unwanted auto-aggressive immune responses by secreting immuno-suppressive cytokines or direct inhibition of target cell function. FoxP3 is a major master transcription factor that plays a central role in regulation of both development and function of Tregs. The balance between Th17 and Treg arms of the immune response is critical for proper and timely resolution of inflammation. The treatment with ivermectin of human CD4⁺ T cells activated under Th17 skewing conditions leads to the up-regulation of FoxP3 mRNA levels whereas Th17-related genes, such IL-17A and RORc (encoding for RORγt transcription factor), were decreased under the same conditions (FIG. 8A). The concurrent regulation of Treg/Th17 genes by ivermectin is followed by 50% increase in the percentage of Foxp3⁺ T cells during the co-culture in vitro (FIG. 8B).

### Example 10—Ivermectin Inhibits IL-17 and IFN-γ Secretion in Human PBMCs Stimulated In Vitro

### Methods

Peripheral blood peripheral blood mononuclear cells (PBMCs) were treated with indicated doses of ivermectin and then stimulated by anti-CD3/CD28 mAbs for 48 hours. The supernatants were analyzed for IL-17 and IFN-γ by ELISA. One representative of three different experiments is shown.

### Results

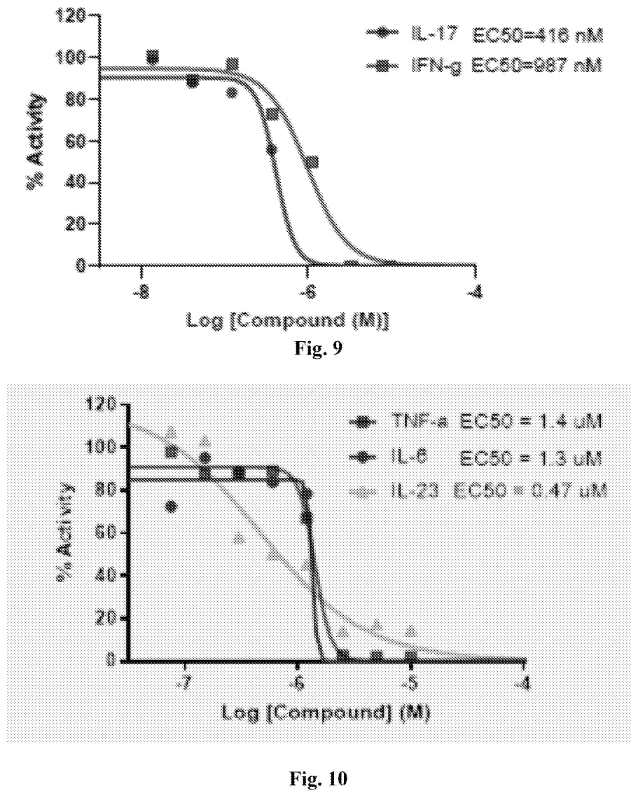

The anti-inflammatory potential of Ivermectin was further confirmed in total peripheral blood peripheral blood mononuclear cells (PBMCs) stimulated by anti-CD3/CD28 antibodies. Treatment of PBMCs with increasing doses of Ivermectin resulted in a dose-dependent decrease of both IL-17 and IFN-γ induced by anti-CD3/CD28 stimulation with EC50s at 416 nM and 987 nM, respectively, as shown in FIG. 9.

### Example 11—Ivermectin Inhibits LPS-Induced Secretion of TNF-α, IL-6, and IL-23 in Human PBMCs

### Methods

PBMCs were treated with indicated doses of ivermectin and then stimulated by LPS (1 mg/mL) for 48 hours. The supernatants were analyzed for TNF-α, IL-6, and IL-23 by ELISA. One representative of three different experiments is shown in FIG. 10.

### Results

To determine the effect of Ivermectin on pro-inflammatory cytokine secretion induced by TLR stimulation. Human PBMC were treated with lipopolysaccharide (LPS) with or without increased doses of Ivermectin. Ivermectin robustly decreased levels of TNF-α, IL-6 and IL-23 induced by LPS activation for 2 days, with EC50s at 1.4 μM, 1.3 μM, and 0.47 μM, respectively, as shown in FIG. 10.

### Example 12—Ivermectin Inhibits IL-1p in TLR-Induced Human Monocytes

### Methods

Human monocytes were isolated from peripheral blood and treated with indicated doses of ivermectin and then stimulated by LPS (100 ng/mL), Pam2CSK4 (100 ng/ml) and Pam3CSK4 (100 ng/ml) for 24 hours. The supernatants were analyzed for IL-1β by ELISA.

### Results

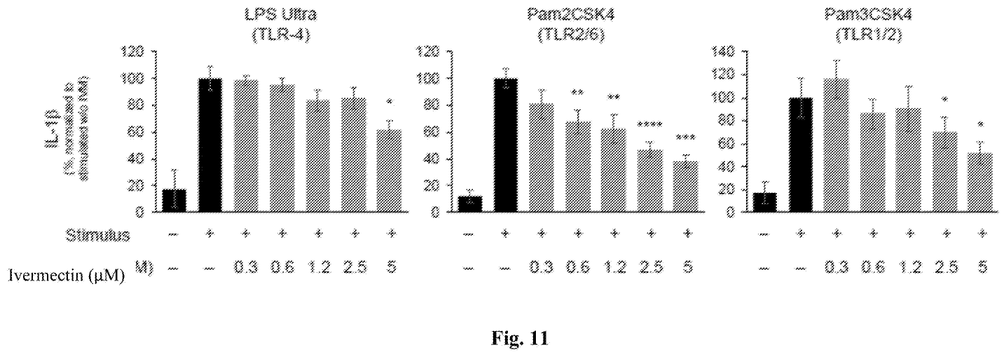

By using purified human monocytes, we found that ivermectin treatment significantly down-regulates IL-1 secretion induced by ultra-pure LPS, Pam2CSK4 and Pam3CSK4 which are TLR4, TLR2/6 and TLR1/2 ligands, respectively (FIG. 11).

### Example 13—Ivermectin Inhibits TNF-α and IL-6 Secretion in Dendritic Cells

### Methods

Monocytes-derived dendritic cells (moDC) were differentiated by culturing purified human monocytes with 25 ng/mL hGM-CSF and 50 ng/mL hTL-4 for 5-7 days to reach full differentiation. Then, moDC were treated with indicated doses of ivermectin and then stimulated by Pam2CSK4 (100 ng/ml) and Pam3CSK4 (100 ng/ml) for 24 hours. The supernatants were analyzed for IL-6 and TNF-α by ELISA. Bar graphs represent pooled data from 2-3 experiments.

### Results

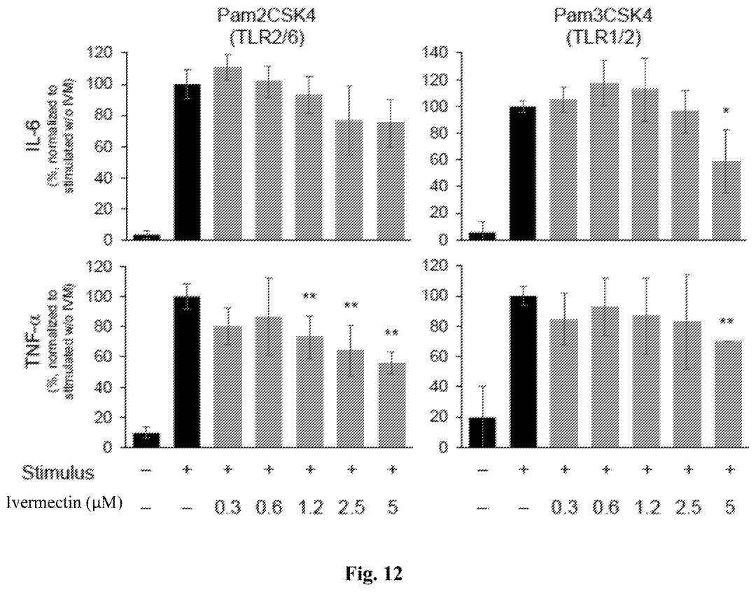

Pro-inflammatory cytokines IL-1β, IL-6 and IL-23 in combination with T-cell receptor (TCR) activation by antigen-presenting cells (APCs), such as dendritic cells, are essential for optimal Th17 differentiation and induction of IL-17 secretion in T cells. Monocytes-derived dendritic cells (moDC) were differentiated by adding cytokines GM-CSF and IL-4 to purified monocytes and culturing cells for 5-7 days. Treatment of moDC with ivermectin significantly and in a dose-dependent manner down-regulates the ability of cells to secrete both TNF-α and IL-6 in response to TLR signaling (FIG. 12), further demonstrating the direct anti-inflammatory potential of ivermectin in innate immune cells.

### Example 14—Patient Dosed Phase 1 Study

To evaluate safety, tolerability, PK, and high-fat food effects on formulation 7E, a Phase 1 double-blind, placebo-controlled, randomized (3:1), dose-finding study was performed in healthy subjects and its effects on quality of life was assessed. A single ascending dose (SAD) portion of the study was followed by a multiple ascending dose (MAD) cohorts at 10, 20, 40, 80 and 120 mg of formulation 7E once a day for 14 days. The study population were healthy individuals over 18-65 years of age.

### SAD Portion of the Study

Participants were admitted to the clinical research center the day before dosing for a baseline assessment, including an eligibility review, physical exam, neurological exam, laboratory evaluations, an EKG, and pupillometry. Subjects followed a fasting dose protocol and were given a single dose administration of the study product followed by serial PK, vital signs, and safety assessments including pupillometry and neurological exams over 120 hours. Additionally, PK sample collection occurred at the following time points: pre-dose (within 2 hours prior to dosing) and at 0.5, 1, 2, 3, 4, 5, 6, 8, 10, 12, 16, 24, 36, 48, 72, and 120 hours post-dose). Each subsequent SAD cohort (see, Table 11) begun 72 hours after the last subject in the previous cohort was dosed, as long as fewer than 2 of the participants in that cohort experienced a dose limiting toxicity (DLT), as ivermectin toxicity manifests within 24 hours of dosing. Participants in the 40 mg cohort of the SAD portion of the study (or the maximum tolerable dose (MTD), whichever dose was lower) were not discharged on day 6 and instead continued onto a fed sub-study, with the fed dose administered 10 days after the fasted dose in conjunction with a high-fat meal. Evaluations following the fed dose were identical to those after the fasted SAD doses and continued through 120 hours.

Two vials of whole blood samples for PD measurements were collected pre-dose and 24 hours after the single dose for exploratory biomarker research.

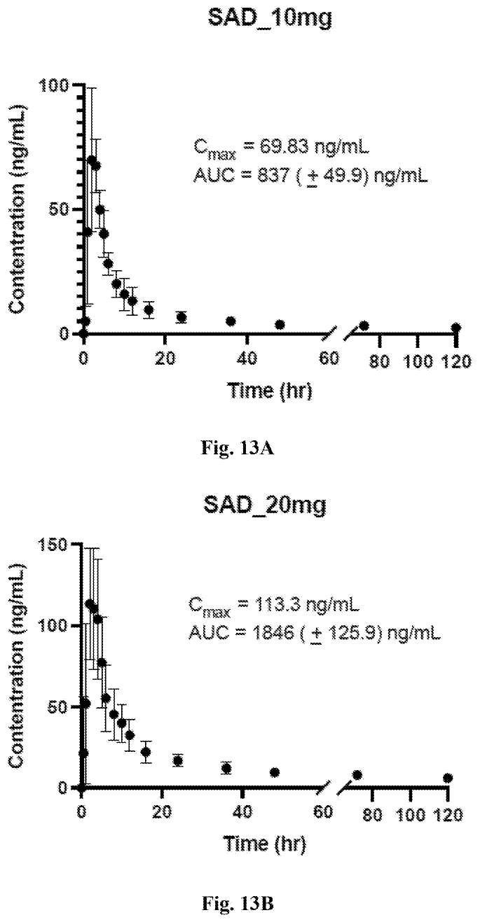

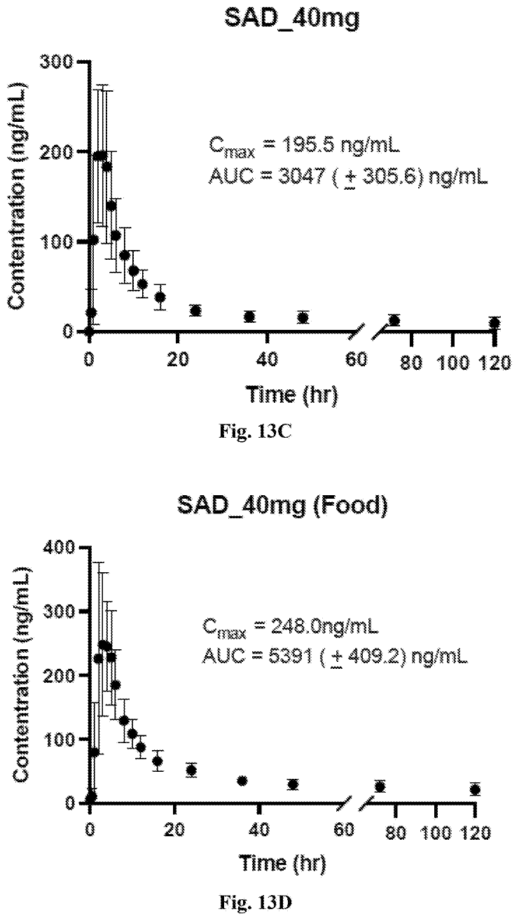

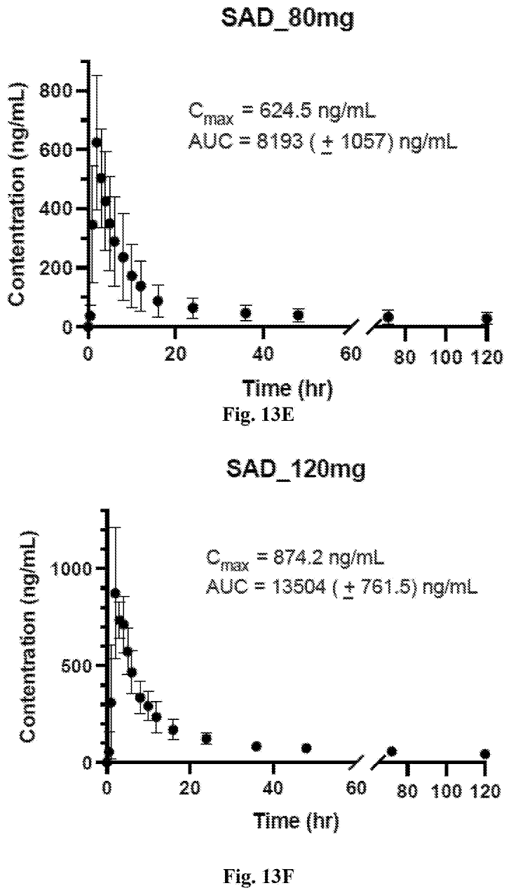

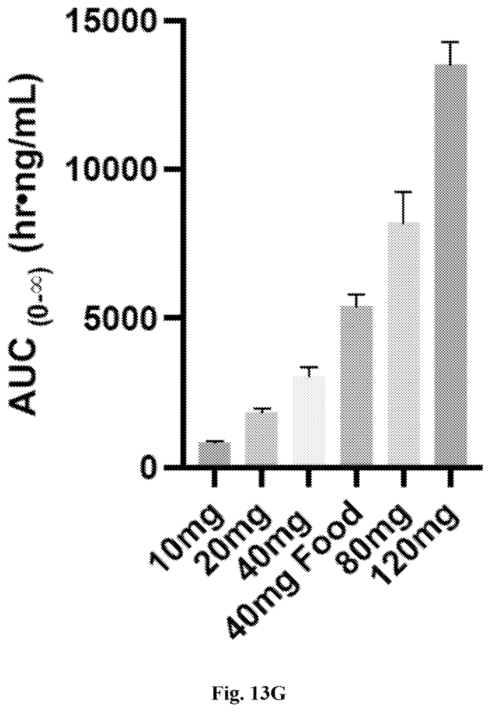

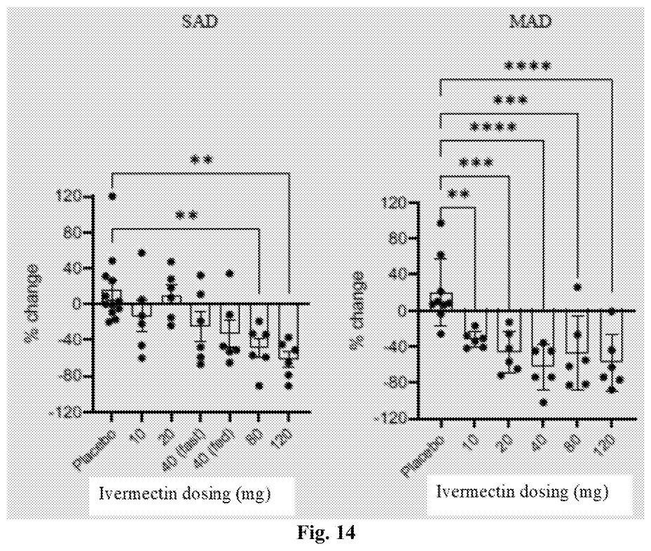

The results of the PK analysis for the SAD portion of the study are shown in FIGS. 13A-G and Table 12.

### MAD Portion of the Study (14-Day Study, One Dose Per Day)

Each MAD cohort 10 mg-80 mg (see, Table 11) begun dosing the day after the SAD cohort at the dose above had completed dosing and had been monitored for 72 hours, provided stopping criteria were not met in the SAD study or in a lower dose MAD cohort. If stopping criteria were not yet met, the 120 mg MAD cohort begun after the last subject in the 80 mg cohort had been dosed through day 5, as steady state was estimated to occur around 4 days.

Volunteers underwent screening and baseline assessments as described above for the SAD portion of the study. Subjects followed a fasting dose protocol on days 1, 2, and 14, and were given a single daily administration of the study product for 14 days. After dosing on days 1, 7 and 14, vital signs and safety assessments including pupillometry and neurological exams were performed. These and additional safety assessments were also performed on day 19. PK sample collection occurred at the following time points after Day 1 and Day 14 dosing: pre-dose (within 2 hour prior to dosing) and at 0.5, 1, 2, 3, 4, 5, 6, 8, 10, 12, 16, 24, 36, 48, 72, and 120 hours (pre-dose) after the initial day 1 dose. Blood for PK was also drawn pre-dose on days 5, 7, 8, 9, 10, 12, and 14. If 2 participants in a cohort experienced a DLT, that cohort was stopped as were any overlapping cohorts at a higher dose.

Two vials of whole blood samples for PD measurements were collected pre-dose and on Day 15 (24 hours after a subject's last dose of study drug in the MAD study) for exploratory biomarker research.

TABLE 11
Phase I Dose Cohorts

| Study | Dose | No. of | Proposed Doses (May |
|---|---|---|---|
| Portion | Cohort | Subjects | Differ Based on CRM Output) |
| SAD | 1 | 8 | Formulation 7E gelcap 10 mg (n = 6) vs placebo (n = 2) |
| SAD | 2 | 8 | Formulation 7E gelcap 20 mg (n = 6) vs placebo (n = 2) |
| SAD | 3 | 8 | Formulation 7E gelcap 40 mg (n = 6) vs placebo (n = 2) |
| SAD | 4 | 8 | Formulation 7E gelcap 80 mg (n = 6) vs placebo (n = 2) |
| SAD | 5 | 8 | Formulation 7E gelcap 120 mg (n = 6) vs placebo (n = 2) |
| MAD | 1 | 8 | Formulation 7E gelcap 10 mg (n = 6) vs placebo (n = 2) |
| MAD | 2 | 8 | Formulation 7E gelcap 20 mg (n = 6) vs placebo (n = 2) |
| MAD | 3 | 8 | Formulation 7E gelcap 40 mg (n = 6) vs placebo (n = 2) |
| MAD | 4 | 8 | Formulation 7E gelcap 80 mg (n = 6) vs placebo (n = 2) |
| MAD | 5 | 8 | Formulation 7E gelcap 120 mg (n = 6) vs placebo (n = 2) |
| Food | 4* | 8 | Formulation 7E gelcap 40 mg (n = 6) vs placebo (n = 2) |
| effect | | | |

*Formulation 7E gelcap 40 mg (or MTD, whichever is lower) (n = the 8 from the 40 mg or MTD SAD cohort)

TABLE 12
PK data for Phase I SAD Portion

| | | | | 40 mg | | |
|---|---|---|---|---|---|---|
| | | | | (with | | 120 |
| SAD Cohort | 10 mg | 20 mg | 40 mg | food) | 80 mg | mg |
| Cₘₐₓ (ng/ml) | 69.83 | 113.3 | 195.5 | 248.0 | 624.5 | 874.2 |
| AUC_{(0-∞)} (ng · | 837 | 1846 | 3047 | 5391 | 8193 | 13504 |
| h/ml) (SD) | (49.86) | (125.9) | (305.6) | (409.2) | (1057) | (761.5) |

### Example 15—Oral Administration of Ivermectin in Human Subjects Down-Regulates IL-17 Secretion in Peripheral Blood Mononuclear Cells (PBMCs) Stimulated Ex Vivo (Phase 1)

### Method

PBMCs were purified from healthy human subjects before and after (24 hours after the last dosing) oral administration of ivermectin at doses 10, 20, 40, 80, 120 mg or placebo, and stimulated for 48 hrs by immobilized monoclonal antibodies (mAbs) against CD3 and CD28 (anti-CD3/28). The supernatants were analyzed for IL-17 by ELISA.

### Results

Data from Phase 1 clinical trial demonstrated that oral administration of ivermectin to healthy subjects down-regulates the ability of T cells to secrete IL-17 in response to TCR stimulation ex vivo. The inhibitory effect of ivermectin on IL-17 secretion was robust and dose dependent, with statistical significance achieved at 80 mg and 120 mg doses of the single ascending dose (SAD) and at all the doses of the multiple ascending doses (MAD) portion of the study (FIG. 13). These data are consistent with profound inhibition of IL-17 secretion by ivermectin treatment of Th17-skewed T cells in vitro (FIG. 6), and further confirm the anti-inflammatory potential of ivermectin capsules in the clinic.

### Example 16—Patient Dosed Phase 2a Study (Daily, 12 Weeks)

To evaluate clinical efficacy, safety, and tolerability of formulation 7E, a randomized, double-blind, placebo-controlled, multicenter study, Phase 2a, dose-finding study was performed in which ivermectin (formulation 7E) was administered at 10 mg, 20 mg, 40 mg, and 60 mg as once-daily adjunctive treatment in adult patients with epilepsy (see Table 13). The population were subjects aged 18 years to 60 years who had been diagnosed with epilepsy according to the International League Against Epilepsy (ILAE) Classification of the Epilepsies 2017 criteria (ILAE 2017) and who were uncontrolled on one to four concomitant antiepileptic drugs (AEDs) at optimal stable dosages for ≥4 weeks prior to screening and throughout the treatment period.

TABLE 13
Phase II Dose Cohorts

| Dose Cohort | No. of Subjects | Doses |
|---|---|---|
| 1 | 8 | Formulation 7E gelcap 10 mg |
| 2 | 8 | Formulation 7E gelcap 20 mg |
| 3 | 10 | Formulation 7E gelcap 40 mg |
| 4 | 9 | Formulation 7E gelcap 60 mg |
| Placebo | 8 | Matching placebo |

### Objectives

The primary objective was to assess the safety of a range of doses of formulation 7E as an adjunctive therapy in subjects with epilepsy.

Secondary objectives included: (1) To assess preliminary efficacy (median % reduction relative to baseline in number of countable observable seizures per 28 days in modified Intent-to-Treat [mITT] populations within the treatment cohorts as compared with placebo) in terms of overall seizure reduction over a range of doses of formulation 7E in subjects with epilepsy; (2) to assess preliminary efficacy (median % reduction relative to baseline in number of countable observable seizures per 28 days in mITT populations within the treatment cohorts as compared with placebo) in terms of seizure reduction by seizure type (focal, generalized, and unknown) over a range of doses of formulation 7E in subjects with epilepsy; (3) to assess preliminary efficacy (median % reduction relative to baseline in number of countable seizures per 28 days in mITT populations within the treatment cohorts as compared with placebo) in terms of reduction of generalized tonic-clonic seizures and focal to generalized tonic-clonic seizures over a range of doses of formulation 7E in subjects with epilepsy; (4) to assess seizure freedom over time; (5) to assess tolerability of formulation 7E over a range of doses in subjects with epilepsy; (6) to assess the effect on quality of life of a range of doses of formulation 7E in subjects with epilepsy; and (7) to assess whether formulation 7E, over a range of doses, impacts suicidality.

Exploratory objectives included: (1) to assess levels of concomitant AEDs overtime; (2) to assess formulation 7E levels as a function of concomitant AEDs; (3) to correlate plasma levels of formulation 7E with safety, tolerability, and quality-of-life measures; (4) to correlate plasma levels of formulation 7E with overall efficacy in reducing seizures; (5) to correlate plasma levels of formulation 7E with efficacy by seizure type (focal, generalized, and unknown); (6) to correlate plasma levels of formulation 7E with plasma levels of biomarkers including IL-17, IL-6, IL-1β, TNF-α, IL-23, IFN-γ, additional exploratory biomarkers; (6) to correlate efficacy in reducing seizures with changes in biomarkers; and (7) additional measures of efficacy.

### Endpoints

Primary endpoints included a safety assessment, namely a comparison of Grade 2 or higher adverse events (AEs) in each dose cohort as compared with placebo.

### Secondary Endpoints Included:

(1) Efficacy: (a) Median change (%) in the number of countable observable seizures overall and by seizure type (focal, generalized, and unknown onset) per 28 days in mITT populations within the treatment cohorts relative to baseline as compared with placebo; (b) median change (%) in the number of countable observable seizures overall and by seizure type (focal, generalized, and unknown onset) per 28 days in mITT populations within the treatment cohorts relative to baseline as compared with placebo after 14 days of study drug use; (c) median change (%) relative to baseline in the number of countable generalized tonic-clonic and focal to generalized tonic-clonic seizures per 28 days in mITT populations within the treatment cohorts relative to baseline as compared with placebo for the duration of treatment and after 14 days of study drug use; (d) change in Quality of Life in Epilepsy—Problems (QOLIE-31-P) scale score at Day 84 as compared with baseline in treated cohorts as compared with placebo; and (e) percent (%) of subjects who are seizure-free by study day(s) 1-84.

(2) Safety: Change in Columbia Suicide Severity Rating Scale (C-SSRS) responses at Day 84 as compared with baseline in treated cohorts as compared with placebo.

(3) Safety and Tolerability: (a) Number of subjects who withdrew from treatment because of study-drug effects; and (b) Number of subjects in each dose cohort who, after the initial 2 weeks, with investigator, decreased their dose of study drug because of treatment-related effects.

(4) Exploratory: (a) Plasma levels of concomitant AEDs over time; (b) formulation 7E plasma levels as a function of concomitant AEDs; (c) correlation of plasma levels of formulation 7E at 2, 4, 8, and 12 weeks of treatment with (i) incidence of Grade 2 or higher adverse events (AEs); (ii) change from baseline in QOLIE-31-P score at 4 and 12 weeks only; (iii) change from baseline in C-SSRS responses at 4 and 12 weeks only; (iv) efficacy in terms of overall seizure reduction and reduction of focal, generalized, and unknown type seizures as compared with baseline periods; (v) efficacy in terms of reduction of generalized tonic-clonic and focal to generalized tonic-clonic seizures as compared with baseline periods; (vi) plasma levels of biomarkers, including IL-17, IL-6, IL-1β, TNF-α, IL-23, IFN-α, and additional exploratory biomarkers (Weeks 4, 8, and 12 only); (d) correlation of plasma levels of biomarkers with efficacy in terms of overall seizure reduction, reduction of specific seizure types (focal, generalized, and unknown) and reduction of generalized tonic-clonic and focal to bilateral tonic-clonic seizures as compared with baseline periods; (e) ≥50% response rate in treated subjects overall and at each dose as compared with placebo during the treatment period; (f) ≥70% response rate in treated subjects overall and at each dose compared with placebo during the treatment period; (g) ≥90% response rate in treated subjects overall and at each dose as compared with placebo during the treatment period; and (h) seizure freedom in treated subjects overall and at each dose as compared with placebo during treatment Weeks 3 to 12 and Weeks 1 to 12.

### Study Design

This study was a double-blind, placebo-controlled, randomized (4:1), safety and dose-finding study of adjunctive formulation 7E in subjects aged 18 years to 60 years who had been diagnosed with epilepsy according to the International League Against Epilepsy (ILAE) Classification of the Epilepsies 2017 criteria (ILAE 2017) and who were uncontrolled on one to four concomitant antiepileptic drugs (AEDs) at optimal stable dosages for >4 weeks prior to screening and throughout the treatment period.

### Eligibility

Inclusion criteria included (1) diagnosed with epilepsy according to ILAE 2017 criteria and with uncontrolled countable seizures (as per Epilepsy Study Consortium review) on one to four concomitant AEDs at optimal stable dosages for at least 4 weeks prior to screening and throughout the treatment period; (2) age 18 to 60 years of age; (3) must have had a brain magnetic resonance imaging (MRI) or computerized tomography (CT) scan with an available report (images need not be available) that is negative for other confounding conditions; and (4) must have an electroencephalogram (EEG) report (read out preferred) consistent with the subject's seizure type(s).

Exclusion criteria included (1) history of hypersensitivity to ivermectin; (2) ivermectin use within 28 days of screening; (3) history of progressive neurological disorder or other significant progressive disorder or unstable medical condition(s); (4) change in AED regimen in the 28 days prior to screening; (5) taking >4 concomitant AEDs at screening; (6) history of status epilepticus in the 2 years prior to screening; (7) history of traumatic brain injury within 28 days prior to screening; (8) history of psychogenic non-epileptic seizures (PNES), active or within 2 years prior to study entry; (9) epilepsy-related surgery within 1 year prior to screening, epilepsy-related radiosurgery or laser surgery within 1 year prior to screening; (10) epilepsy dietary therapy initiated <3 months prior to screening; (11) administration of investigational product in another trial within 28 days prior to the first expected study drug administration, or five half-lives, whichever is longer; (12) receiving felbamate for <1 year prior to screening; (13) receiving vigabatrin for <2 years prior to screening. Subjects on vigabatrin should have available, appropriate documentation of visual fields; (14) receiving ezogabine (ex-US) at screening; (15) use of the following medications and foods at screening or baseline that may interfere with study drug metabolism (please note AEDs with CYP3A4 metabolism are not excluded from this study as drug levels and safety are monitored throughout the study): (a) CYP3A4 inducers: rifampin, lumacaftor, mitotane, enzalutamide, apalutamide, St. John's wort, glucocorticoids; (b) CYP3A4 inhibitors including and not limited to: clarithromycin, ceritinib, idelalisib, lonafarnib, tucatinib, erythromycin, telithromycin, diltiazem, ketoconazole, posaconazole, voriconazole, telithromycin, nefazodone, mifepristone, itraconazole, ketoconazole, anti-retroviral drugs (atazanavir, darunavir, indinavir, lopinavir, nelfinavir, ritonavir, saquinavir, tipranavir), grapefruit and grapefruit juice, pomegranate and pomegranate juice; (c) additional medications that may interact with CYP3A4, PGP, or Vitamin K: fluconazole, isavuconazole, cyclosporine, amiodarone, dronaderone, verapamil, imatinib, warfarin, acenocoumarol.

### Methodology

Forty-three evaluable male or female eligible subjects were sequentially enrolled in up to 4 dose cohorts and, within each cohort, randomized with a 4:1 ratio (formulation 7E to placebo). Before dosing, all subjects underwent a screening assessment, followed by a 4-week or longer prospective baseline assessment period to assess seizure frequency.

This dose-finding study used the continuous reassessment method (CRM) of O'Quigley et al. to determine the dose of formulation 7E to be used for each subject cohort (O'Quigley J, Pepe M, & FisherL. Continual Reassessment Method: A Practical Design for Phase 1 Clinical Trials in Cancer. Biometrics. 1990;46(1), 33-48, incorporated by reference herein in its entirety). Cohorts were dosed sequentially beginning with Dose Cohort 1. This study initially randomized a total of 10 subjects into the first dosing cohort (10 mg daily, 4:1 active to placebo) for 12 weeks. Safety data were reviewed after 14 days to determine whether the next cohort can be opened. Dosing continued through the CRM, with oversight by the Safety Review Committee (SRC) after each cohort, until 10 subjects had completed 14 days of dosing at 60 mg daily or until a total of 10 subjects had received the same active dose for 14 days, and this dose was identified as the maximum tolerated dose (MTD). A 1000-trial simulation using a prior skeleton of standardized dose toxicity probabilities of 0.01, 0.05, 0.10, and 0.20 suggested the MTD would be identified with 32 subjects receiving the experimental drug.

In each dosing cohort, subjects with no dose-limiting toxicities (DLTs) during the initial 14 days of treatment continued with dosing through 12 weeks, as long as they did not experience a DLT, meet other withdrawal criteria, or decided to withdraw from treatment. Evaluations of PK parameters, together with safety, efficacy, and concomitant AEDs, were made after 2, 4, 8, and 12 weeks of treatment by an unblinded study statistician and provided to a sponsor-designated study physician for review. This review was performed separately from the review for dose escalation, which was based on DLT development. Subjects, observers, and analysts of the final study data remained blinded.

Over the course of their participation, subjects came to the clinic for 7 visits and also had 4 telephone interviews. During the study, subjects underwent medical history evaluations; physical and neurological examinations; vital sign measurements; AE assessments; QOLIE-31-P and C-SSRS surveys; concomitant medication assessments; blood sample collection for PK, hematology (including coagulation), concomitant AED levels, chemistry; urinalysis; and electrocardiogram (ECG).

Open-label extension: Once the 12-week study dosing period is complete, all subjects who continue to meet eligibility may enroll in an open-label extension, during which period investigators may make dose adjustments down to 10 mg and up to the dose at which a cohort has not met stopping criteria and has completed at least 14 days of dosing. In the event that a cohort is stopped for toxicity, subjects may elect to enter the open-label extension (OLE) at up to the dose below the stopped cohort. Modifications to concomitant AEDs are allowed during the OLE and must be documented on the appropriate CRF. During this OLE, subjects will have in-person visits every 8 weeks and telephone interviews every 8 weeks for the first 6 months, so that there is follow-up every 4 weeks. After the initial 6 months, in-person visits will be conducted every 13 weeks. Subject safety, tolerability, and efficacy data will be collected as in the SOA. Data is continuously monitored for safety and efficacy with safety and efficacy reports generated at least quarterly. The initial data cut will be 1 year from the Last Patient Last Visit (LPLV) in the double-blind portion of the study with cuts annually thereafter.

Efficacy outcomes include the median percent change in focal seizure frequency at consecutive six-month intervals as compared with baseline and ≥50%, ≥70%, ≥90% and 100% responder rates at consecutive 12-month intervals overall and by dose (dose during the majority of the period will be used in cases when the dose in not constant over that time). Tolerability will be reported descriptively as the percent of subjects who discontinue treatment during the first 3 months, and then at 6 months, 12 months and each 12-month interval ongoing. Dose changes, duration and median daily dose will be reported descriptively at 3 months, and then at 6 months, 12 months and each 12-month interval ongoing. Safety assessments include frequency and severity of TEAEs and C-SSRS responses and will be reported descriptively, per dose, including those that lead to discontinuation, at 3 months, 6 months, 12 months, and consecutive 12-month intervals.

Follow-up: Follow-up visits will occur 30 days (±3 days) after the last dose of study drug for subjects who choose not to continue into the open-label extension. Subjects will undergo the following safety assessments: physical exam; neurological exam; vital sign measurements; AE assessments; QOLIE-31-P and C-SSRS surveys; concomitant medication assessments; blood sample collection for hematology (including coagulation), concomitant AED levels, chemistry; and urinalysis. If another therapy is started within 30 days after the last dose of study drug, the Follow-up Visit will be conducted before the start of the other therapy.

### Diagnosis and Main Criteria for Inclusion:

Participants 18 to 60 years of age who were diagnosed with epilepsy, as defined by the ILAE 2017 guidelines, who had observable, countable seizures that were uncontrolled on one to four concomitant AEDs at optimal stable dosages for >4 weeks prior to screening and throughout the treatment period. Subjects had to have 3 seizures per 4 weeks in the baseline period prior to randomization. Acceptable seizure types included generalized, focal, or of unknown onset, but did not include absence seizures or focal aware seizures without a detectable motor component, aphasia, or other observable symptom.

### Investigational Product, Dosage and Mode of Administration

Formulation 7E was supplied as 10 mg and 20 mg soft gel capsules and administered once daily orally at 10, 20, 40, or 60 mg.

### Duration of Treatment

12 weeks with the option for open-label extension for all subjects.

### Reference Therapy, Dosage and Mode of Administration

Matching placebo capsules (10 mg and 20 mg sizes) administered once daily orally.

### Dose Escalation

After 14 days of dosing in a cohort, safety data were processed per CRM and be reviewed by the SRC which then issued a recommendation regarding dose escalation. Determination of dose escalation was based on the development of DLTs in previously treated subjects according to the CRM design. Ten (10) subjects were dosed within each dose cohort, eight with formulation 7E and two with placebo, with an additional assignment of 2 active-drug subjects at the discretion of the CRM and SRC. There was a total of up to 4 dose cohorts, for a sample size of 43.

If 2 active-treatment subjects in a cohort developed a DLT in the initial 14 days, that cohort dose was stopped. If stopping criteria were met after the initial two-week evaluation period, treatment was stopped for that individual and the cohort will continue with close monitoring by the Medical Monitor, Clinical Trial Lead and the SRC. DLTs included the following: (1) Mydriasis, defined as the absence of pupillary response to light (confirmed with repeat test in 30-60 min); (2) new onset abnormal heel to toe test (confirmed with repeat test in 30-60 min); (3) change from baseline visit in QTc of >60 msec or total QTc≥500 msec; (4) any of the following hepatic abnormalities with no apparent alternative causes (including but not limited to viral hepatitis, acute or chronic liver disease, concomitant administration of known hepatotoxic drug(s)) for the finding (i) alanine transaminase (ALT) or aspartate transaminase (AST)>8×upper limit of normal (ULN); (ii) ALT or AST>5×ULN with confirmation at approximately 2 weeks; (iii) ALT or AST>3×ULN and (total bilirubin>2×ULN or INR>1.5) without findings of cholestasis (elevated ALP); (iv) ALT or AST>3×ULN with appearance of fatigue, nausea, vomiting, right upper quadrant pain or tenderness, fever, rash and or eosinophilia (>5%); (5) two or more similar treatment-related serious or severe AEs within the same system organ class (SOC).

### Study Duration

At least 16 weeks (4-week or longer baseline plus 12-week treatment) for each subject with the option for open-label extension. The duration of the 12-week dosing portion of the study was expected to be approximately 1 year. For subjects who opted in, the OLE continued after the 16-week study period.

### Criteria for Evaluation

Efficacy: Participants or a dedicated observer kept a seizure diary during the baseline and treatment periods. Seizure diary information was monitored at each study visit except Visits 3 and 4, which were safety follow-up telephone interviews (after initial dosing). Changes in the number of observable, countable seizures during the treatment period as compared with the baseline period were used to evaluate overall efficacy and efficacy for various seizure types.

Safety: Participants were monitored for adverse events at every study visit. In the case of a severe or life-threatening event, participants were instructed to first call emergency services, then the investigator, and then their regular physician. In cases of mild or moderate adverse events between visits, participants were instructed to inform the study contact. Safety assessments included AEs, severe AEs (SAEs), neurological exams, physical exams, vital sign measurements, clinical safety laboratory evaluations, ECGs, the C-SSRS responses, and reasons for treatment discontinuations due to toxicity. Vital sign measurements, including blood pressure, pulse rate, respiratory rate, and temperature, were monitored throughout the study, as will neurological examinations. The National Cancer Institute Common Terminology Criteria for Adverse Events (NCI CTCAE; Version 5.0) was used for grading clinical and laboratory AEs. In cases where laboratory abnormalities or other potential AEs were not graded in the CTCAE, the principal investigator (PI) had the authority to grade accordingly. The AE reporting period for a subject begun when the subject provided informed consent, and was continued for 30 days after the last dose of study drug. All AEs that occurred in consented subjects during the AE reporting period were recorded, regardless of the relationship of the AE to study drug. Those that occurred prior to drug dosing were considered medical history. Any known untoward event that occurred beyond the AE reporting period that the investigator assessed as possibly related to study drug was also recorded.

Pharmacokinetics: Pre-dose blood samples were collected from subjects for determination of serum concentrations of formulation 7E on Days 1, 7, 14 (±2 days), 28 (±4 days), 56 (±4 days), and 84 (±4 days).

Pharmacodynamics: Pre-dose whole blood samples were collected from subjects for biomarker analysis on Days 1, 14 (±2 days), 28 (±4 days), 56 (±4 days), and 84 (±4 days). Plasma levels of biomarkers, including IL-17, IL-6, IL-1β, TNF-α, IL-23, IFN-α, and additional exploratory biomarkers, were performed using validated procedures and methods.

### Study Results

The study included a total of forty-three (43) randomized and treated subjects in the safety and mITT populations. Although generalized and unknown seizures were allowed to be included, the total number of subjects with generalized seizures during the treatment period was 1 and with unknown seizures was 0, so these subsets were not analyzed. Subjects had an average age of 40.4±13.14 years, and were on one, two, three, or four (9%, 33%, 40%, 19%, respectively) stable background AMSs, and had failed a median of X previous ASMs prior to study entry. The median baseline seizure frequency across the study groups was 12 per 4 weeks (range 3-123). All 37 subjects who completed the double-blind period entered the open-label extension to evaluate the long-term safety, tolerability, and effectiveness of formulation 7E.

### Key Efficacy Findings

The trial demonstrated that formulation 7E is both safe and well tolerated at all tested doses and showed a dose-dependent decrease in monthly (defined as 28 days) focal seizure frequency when compared to placebo. Dose escalations using the CRM design did not detect a MTD and the 60 mg dose was identified as the Recommended Phase 2 Dose (RP2D). Moreover, the placebo-controlled 12-week extension period provided efficacy data.

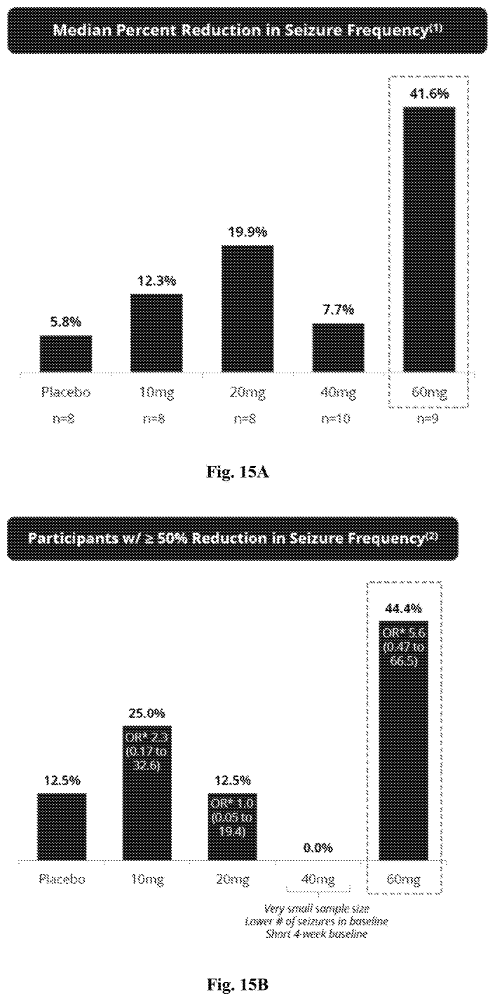

The primary objective of the study was to assess the dose response trend of formulation 7E in reducing monthly focal seizure frequency (FIG. 15A). These data demonstrated a highly statistically significant dose-response relationship for formulation 7E in the adjunctive treatment of focal seizures in adult patients with a history of difficult-to-treat seizures.

A key secondary endpoint of the study was a responder analysis, which compared the proportion of study subjects treated with formulation 7E who achieved a ≥50% reduction in monthly focal seizures versus placebo (FIG. 15B).

### Key Safety and Tolerability Findings

Formulation 7E was well-tolerated in this study with no significant adverse events (SAEs) and all adverse events (AEs) grade 1 or 2. TEAEs were similar in the placebo arm (87.5% of participants) as compared with each dose arm (up to 70% of participants in the 40 mg treatment arm). Neurological AEs were uncommon and, as with all AEs in this study, occurred in <10% of treated participants. Dizziness occurred in 8.6% of treated participants, 0% of participants in the 60 mg treatment arm, and 25% of participants in the placebo arm. Fatigue occurred in 8.6% of treated participants, 22% of participants in the 60 mg treatment arm, and 25% of participants in the placebo arm.

A comparison of 7E with other select AEDs is shown in Table 14.

TABLE 14
Efficacy of 7E as compared to select AEDs

| | | | KEPPRA | VIMPAT | APTIOM | BRIVIACT | XCOPRI |
|---|---|---|---|---|---|---|---|
| | 7E | XEN1101 | (Leviteracetam) | (Lacosamide) | (Eslicarbazepine) | (Brivaracetam) | (Cenobamate) |
| Mechanism of | Anti- | KV7 | SV2A | Na⁺ channel | Na⁺ channel | SV2A | Na⁺ channel |
| Action | inflammatory | channel | | | | | |
| Indication | Adult focal | Adult focal | Adult/Pediatric | Adult/Pediatric | Adult/Pediatric | Adult/Pediatric | Adult |
| | onset seizures | onset | (>1 month) | (>4 years) | (>4 years) | (>16 years) | Focal epilepsy |
| | Adjunctive | seizures | Focal epilepsy | Focal epilepsy | Focal epilepsy | Focal epilepsy | Mono/Adjunct |
| | | Adjunctive | Mono/adjunct | Mono/adjunct | Mono/adjunct | Adjunct | |
| Baseline | 12.0 | 13.5 | — | 11.5 | 8.0 | 9.0 | 8.5 |
| Seizure | per 28 day | per 28 day | | per 28 day | per 28 day | per 28 day | per 28 day |
| Frequency | | | | | | | |
| Placebo- | 60 mg: | 25 mg: | 3000 mg: | 400 mg: | 2000 mg: | 200 mg: | 4200 mg: |
| adjusted | 35% | 35% | 23-31% | 15-29% | 14-24% | 26% | 31% |
| Median | | | | | | | |
| Reduction in | | | | | | | |
| Focal Seizure | | | | | | | |
| Frequency | | | | | | | |
| Placebo- | 60 mg: | 25 mg: | 3000 mg: | 400 mg: | 1200 mg: | 200 mg: | 400 mg: |
| adjusted >50% | 32% | 40% | 29% | 17% | 20% | 18% | 39% |
| Focal | | | | | | | |
| Seizure | | | | | | | |
| Reduction | | | | | | | |

### Key Biomarkers Findings

Formulation 7E (60 mg) significantly decreased IL-17, IL-21, IFN-γ and TNF-α secretion induced in PBMCs by ex vivo stimulation after 12 weeks of treatment. Strong correlations were observed between changes in seizure frequency and the PBMCs secretion of pro-inflammatory cytokines. The spontaneous secretion of IL-1β and TNF-α was increased in placebo, but not in formulation 7E-treated (60 mg) patients. The plasma levels of IL-17 were significantly reduced in formulation 7E-treated (60 mg) patients compared to the pre-dose levels.

### Biomarker Findings

### Experimental Design Methods

Whole peripheral blood samples from clinical trial subjects (6 treated and 2 placebo subjects per cohort) were collected at Day 0 before dosing begins (pre-dose/baseline) as well as after 2, 4, 8, and 12 weeks of treatment with 60 mg of formulation 7E. PBMCs were purified by Ficoll-Paque and cryopreserved until ex vivo analysis. On the day of the experiment, cells from each sample were thawed and allowed to rest overnight in complete tissue culture media in a 37° C. incubator. Then, cells were counted, resuspended at 1×10⁶/ml, and cultured on anti-CD3 mAb (5 μg/ml) and anti-CD28 mAb (5 μg/ml) pre-coated plates. IL-17, IL-21, IFN-γ, and TNF-α secretion in supernatants was determined by ELISA after 48 hours. Some PBMCs were left unstimulated or stimulated by LPS (TLR4 agonist) at 100 ng/ml, or Pam2CSK4 (TLR2/TLR6 agonist) at 100 ng/ml, and IL-1β, IL-6, and TNF-α secretion in the supernatants was determined by ELISA after 24 hours. Exact cytokine concentration was calculated by using standard curve. Standard Deviation is indicative of duplicates for the same sample. % change was calculated as (after-dosing value/pre-dosing −1)×100. Statistical analysis was performed as indicated in the figure legends.

### Results—IL-17, IL-21, IFN-γ, TNF-α Secretion

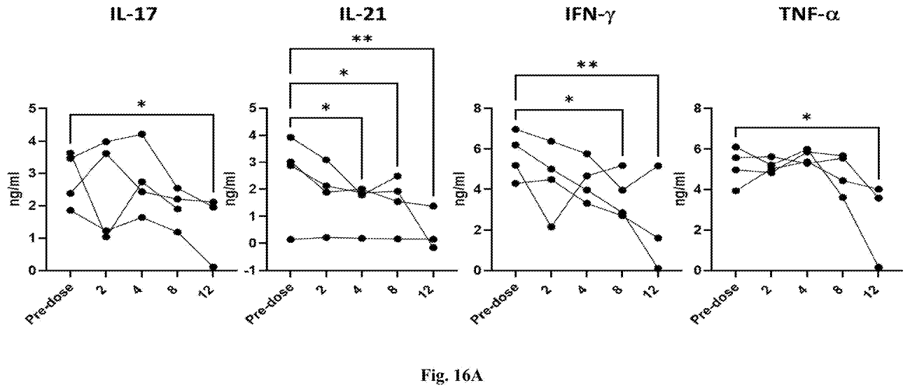

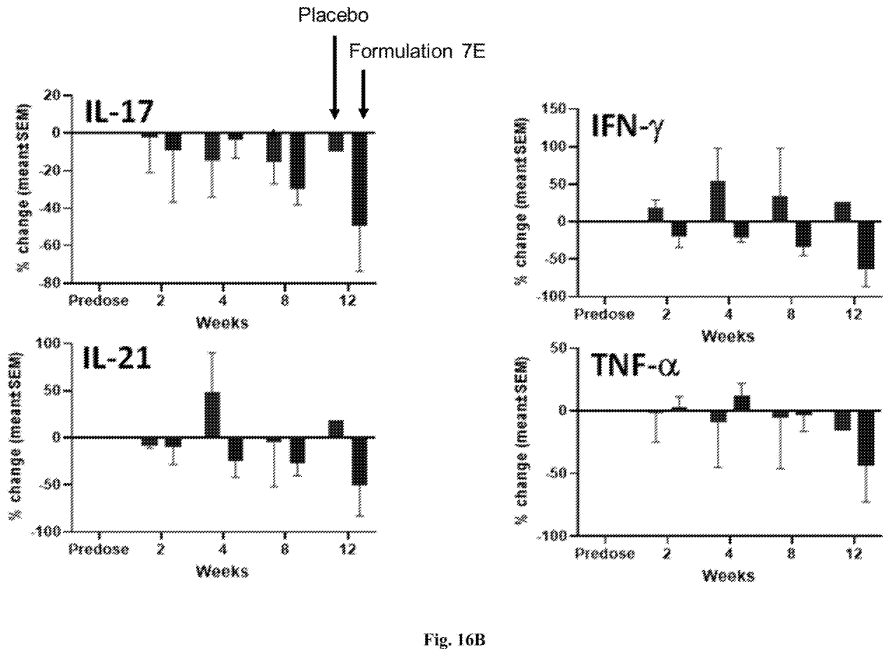

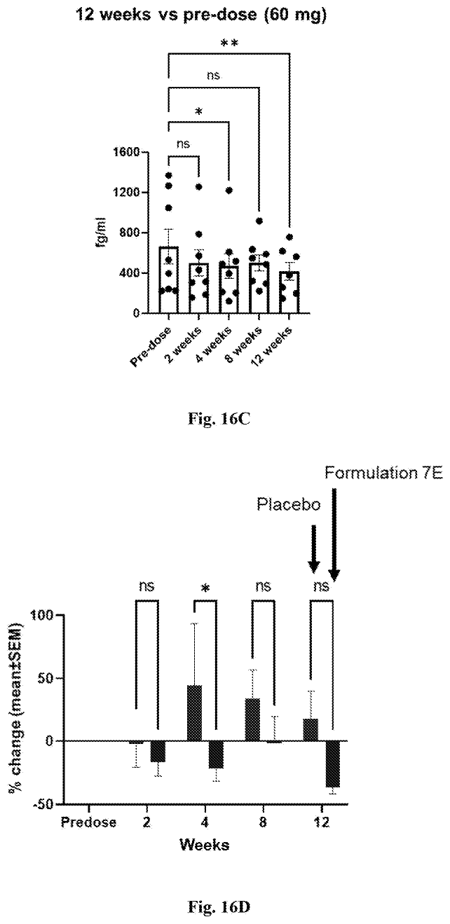

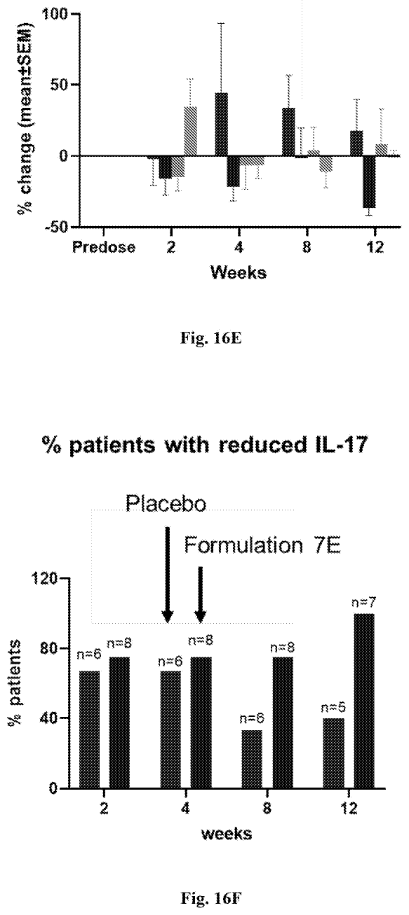

The IL-17, IL-21, IFN-γ, TNF-α secretion was detected in all the samples of ex vivo stimulated PBMCs purified from patients with epilepsy participating in the clinical trial. The IL-17, IL-21, IFN-γ, TNF-α levels ranged between 0.11-4.21, −0.15-3.93, −0.16-6.97, 0.17-6.20 ng/ml respectively. As shown in FIGS. 16A and 16B, oral administration of 60 mg of formulation 7E in patients with epilepsy for 12 weeks significantly down-regulated pro-inflammatory cytokine secretion induced in PBMCs in response to T-cell receptor (TCR) stimulation ex vivo. In addition, the reduction in pro-inflammatory cytokine secretion (compared to pre-dose) over time was more profound in PBMC samples from formulation 7E-treated (60 mg) than in placebo-treated subjects. These data are consistent with in vitro data demonstrating robust inhibition of pro-inflammatory cytokine secretion by formulation 7E treatment of Th17-skewed T cells and further confirming the anti-inflammatory potential of formulation 7E in patients with epilepsy. FIGS. 16C, 16D, 16E, and 16F show a summary of the results obtained for IL-17.

Results—IL-6, TNF-α and IL-1β secretion

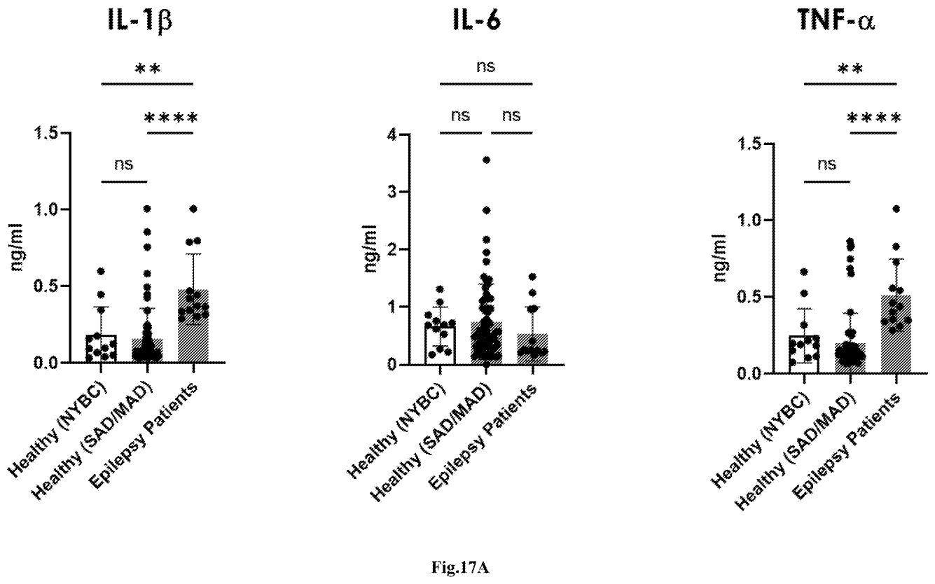

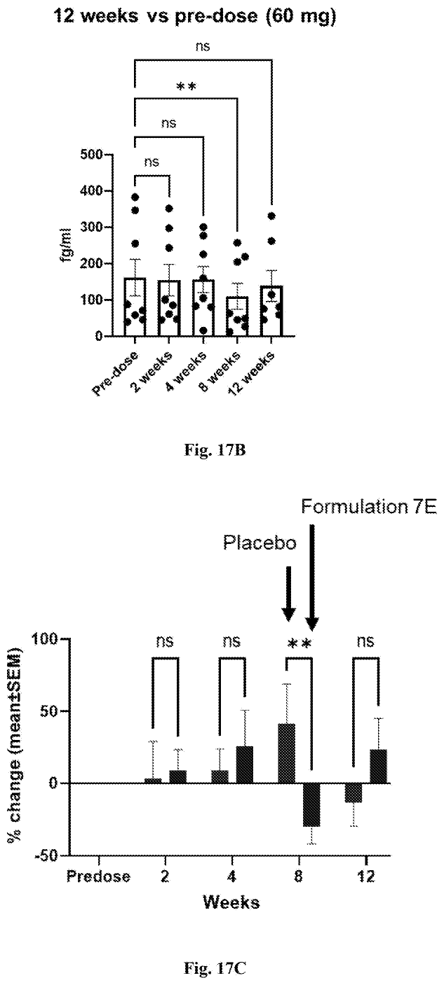

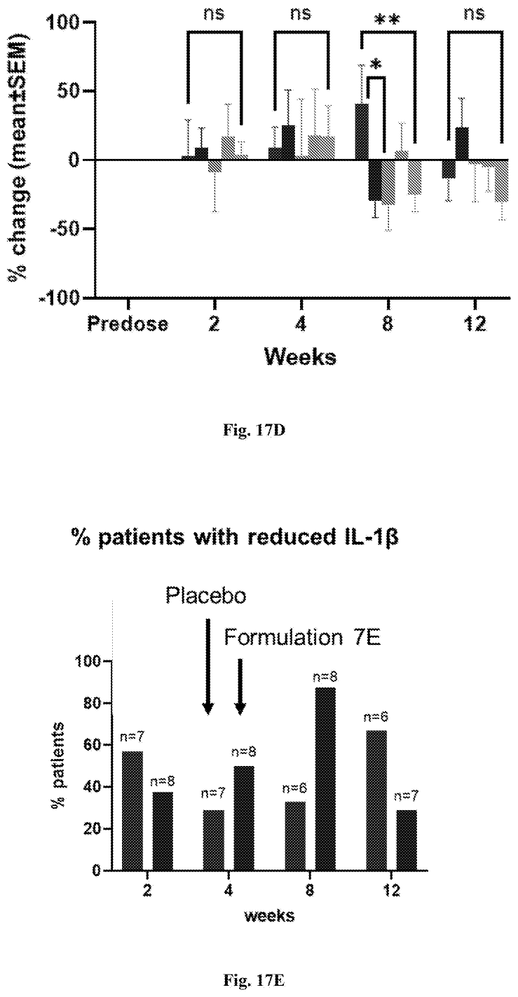

The supernatants collected from unstimulated PBMCs cultured for 24 hours contained detectable levels of pro-inflammatory cytokines, such as IL-6, TNF-α and IL-1β ranging from 0.21-0.42, 0.27-0.56 and 0.29-0.65 ng/ml respectively (Table 15), likely reflecting PBMC basal secreting activity in vivo. Of note, PBMCs from epileptic patients secrete significantly higher levels of IL-1β and TNF-α (FIG. 17A). FIGS. 17B, 17C, 17D, and 17E shows a summary of the results obtained for IL-1β.

As shown in Tables 16 and 17, the ex vivo stimulation of PBMCs with LPS or Pam2CSK4 robustly increased the levels of pro-inflammatory cytokines: LPS-stimulated PBMCs secreted 0.66-11.51, 0.58-8.4 and 0.34-1.98 ng/ml of IL-6, TNF-α and IL-1β respectively, whereas Pam2CSK4-stimulated PBMCs secreted 0.33-3.17, 0.35-1.14 and 0.27-0.76 ng/ml of IL-6, TNF-α, and IL-1β respectively.

TABLE 15
IL-6, TNF-α and IL-1β secretion in unstimulated PBMCs
from patients with epilepsy on Day 1 before dosing begins (Pre-
dose) and after 2, 4, 8, and 12 weeks of formulation 7E treatment
(60 mg) or placebo (marked by *). The supernatants were collected
after 24 hours and analyzed by ELISA. The numbers are indicative
of average from duplicates for the same sample.

| | | | | | |
|---|---|---|---|---|---|
| Patient | Predose | Week 2 | Week 4 | Week 8 | Week 12 |

IL-6 (ng/mL), Unstimulated PBMC

| | | | | | |
|---|---|---|---|---|---|
| 17-005 | 0.41 | 0.31 | 0.36 | 0.42 | 0.32 |
| 17-006* | 0.26 | 0.40 | 0.41 | 0.37 | 0.37 |
| 21-001 | 0.26 | 0.24 | 0.24 | 0.24 | N/A |
| 22-004 | 0.25 | 0.23 | 0.30 | 0.38 | 0.30 |
| 22-005 | 0.23 | 0.20 | 0.21 | 0.27 | 0.24 |
| 22-006* | 0.21 | 0.29 | 0.39 | 0.31 | N/A |

TNF (ng/mL), Unstimulated PBMC

| | | | | | |
|---|---|---|---|---|---|
| 17-005 | 0.56 | 0.43 | 0.56 | 0.44 | 0.40 |
| 17-006* | 0.40 | 0.43 | 0.48 | 0.42 | 0.41 |
| 21-001 | 0.35 | 0.29 | 0.29 | 0.30 | N/A |
| 22-004 | 0.28 | 0.27 | 0.28 | 0.29 | 0.27 |
| 22-005 | 0.35 | 0.30 | 0.31 | 0.32 | 0.31 |
| 22-006* | 0.31 | 0.32 | 0.33 | 0.32 | N/A |

IL-1β (ng/mL), Unstimulated PBMC

| | | | | | |
|---|---|---|---|---|---|
| 17-005 | 0.44 | 0.36 | 0.41 | 0.38 | 0.31 |
| 17-006* | 0.29 | 0.31 | 0.36 | 0.52 | 0.47 |
| 21-001 | 0.37 | 0.35 | 0.36 | 0.32 | N/A |
| 22-004 | 0.30 | 0.31 | 0.41 | 0.40 | 0.42 |
| 22-005 | 0.37 | 0.34 | 0.31 | 0.46 | 0.38 |
| 22-006* | 0.32 | 0.55 | 0.65 | 0.53 | N/A |

TABLE 16
Pro-inflammatory cytokine secretion induced by ex vivo stimulation
with LPS of PBMCs from patients with epilepsy on Day 1
before dosing begins (Pre-dose) and after 2, 4, 8, and
12 weeks of formulation 7E treatment (60 mg) or placebo
(marked by *). The supernatants were collected 24 hours
after stimulation by LPS (100 ng/ml) or Pam2CSK4 (100 ng/ml),
and analyzed by ELISA. The numbers are indicative of average
from duplicates for the same sample.

| | | | | | |
|---|---|---|---|---|---|
| Patient | Predose | Week 2 | Week 4 | Week 8 | Week 12 |

IL-6 (ng/mL), Stimulated PBMC

| | | | | | |
|---|---|---|---|---|---|
| 17-005 | 7.32 | 8.06 | 8.57 | 8.33 | 0.66 |
| 17-006* | 3.36 | 4.46 | 5.53 | 6.36 | 8.26 |
| 21-001 | 3.84 | 11.51 | 7.80 | 7.43 | N/A |
| 22-004 | 3.57 | 3.82 | 3.95 | 4.30 | 6.24 |
| 22-005 | 5.24 | 4.37 | 4.00 | 6.55 | 4.09 |
| 22-006* | 5.73 | 6.80 | 5.57 | 5.16 | N/A |

TNF (ng/mL), Stimulated PBMC

| | | | | | |
|---|---|---|---|---|---|
| 17-005 | 3.48 | 4.35 | 4.34 | 2.91 | 0.58 |
| 17-006* | 1.74 | 2.20 | 2.70 | 2.83 | 4.83 |
| 21-001 | 2.97 | 8.40 | 4.73 | 5.87 | N/A |
| 22-004 | 1.73 | 1.69 | 1.52 | 1.40 | 2.07 |
| 22-005 | 3.87 | 3.42 | 3.76 | 3.91 | 4.88 |
| 22-006* | 3.27 | 2.96 | 2.76 | 2.54 | N/A |

IL-1β (ng/mL), Stimulated PBMC

| | | | | | |
|---|---|---|---|---|---|
| 17-005 | 0.66 | 0.84 | 0.95 | 0.91 | 0.34 |
| 17-006* | 0.38 | 0.46 | 0.77 | 0.95 | 1.48 |
| 21-001 | 0.59 | 1.98 | 0.63 | 0.65 | N/A |
| 22-004 | 0.47 | 0.60 | 0.88 | 0.46 | 1.24 |
| 22-005 | 0.60 | 0.63 | 0.60 | 1.03 | 0.52 |
| 22-006* | 0.73 | 0.89 | 1.00 | 0.86 | N/A |

TABLE 17
Pro-inflammatory cytokine secretion induced by ex vivo stimulation
with Pam2CSK4 of PBMCs from patients with epilepsy on Day
1 before dosing begins (Pre-dose) and after 2, 4, 8, and
12 weeks of formulation 7E treatment (60 mg) or placebo
(marked by *). The supernatants were collected 24 hours
after stimulation by LPS (100 ng/mL) or Pam2CSK4 (100 ng/ml),
and analyzed by ELISA. The numbers are indicative of average
from duplicates for the same sample.

| | | | | | |
|---|---|---|---|---|---|
| Patient | Predose | Week 2 | Week 4 | Week 8 | Week 12 |

IL-6 (ng/mL), Pam2CSK4 stimulated PBMC

| | | | | | |
|---|---|---|---|---|---|
| 17-005 | 1.84 | 2.28 | 3.17 | 2.27 | 0.37 |
| 17-006* | 0.70 | 0.55 | 0.79 | 1.01 | 1.06 |
| 21-001 | 0.87 | 2.18 | 1.38 | 1.69 | N/A |
| 22-004 | 0.62 | 1.10 | 0.58 | 0.66 | 0.60 |
| 22-005 | 0.54 | 0.47 | 0.56 | 0.51 | 0.42 |
| 22-006* | 0.33 | 0.52 | 0.61 | 0.49 | N/A |

TNF (ng/mL), Pam2CSK4 stimulated PBMC

| | | | | | |
|---|---|---|---|---|---|
| 17-005 | 0.75 | 0.96 | 1.14 | 0.75 | 0.43 |
| 17-006* | 0.46 | 0.41 | 0.46 | 0.45 | 0.47 |
| 21-001 | 0.88 | 0.90 | 0.58 | 0.58 | N/A |
| 22-004 | 0.37 | 0.47 | 0.35 | 0.35 | 0.38 |
| 22-005 | 0.41 | 0.41 | 0.41 | 0.38 | 0.40 |
| 22-006* | 0.38 | 0.35 | 0.35 | 0.36 | N/A |

IL-1β (ng/mL), Pam2CSK4 stimulated PBMC

| | | | | | |
|---|---|---|---|---|---|
| 17-005 | 0.42 | 0.38 | 0.56 | 0.50 | 0.32 |
| 17-006* | 0.27 | 0.28 | 0.38 | 0.57 | 0.54 |
| 21-001 | 0.37 | 0.42 | 0.40 | 0.34 | N/A |
| 22-004 | 0.28 | 0.32 | 0.51 | 0.37 | 0.41 |
| 22-005 | 0.39 | 0.38 | 0.35 | 0.48 | 0.39 |
| 22-006* | 0.32 | 0.59 | 0.76 | 0.56 | N/A |

### Changes in Cytokine Secretion at Different Time Points

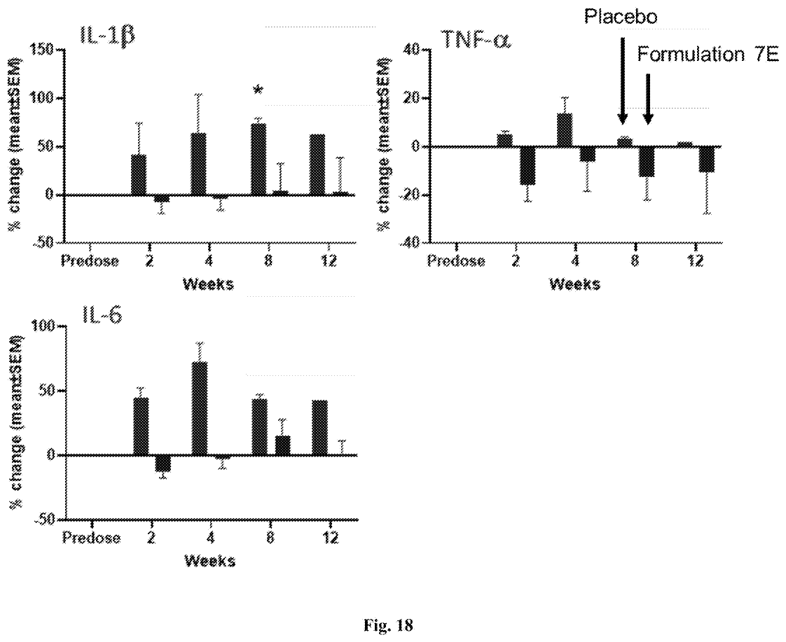

When analyzing the changes in cytokine secretion at different time points compared to pre-dose, it was observed that administration of formulation 7E (60 mg) in patients with epilepsy limited the ability of unstimulated PBMCs to secrete pro-inflammatory cytokines ex vivo compared to placebo (FIG. 18). While IL-1β and IL-6 secretion tended to increase over time in unstimulated PBMCs from placebo patients, secretion remained overall stable in PBMCs from formulation 7E-treated (60 mg) patients compared to pre-dose, with a significative difference reached for IL-1β at week 8. Consistently, TNF-α secretion trended down in PBMCs from formulation 7E-treated patients while it overall remained unchanged in PBMCs from placebo patients. These results altogether suggest that treatment with 60 mg formulation 7E may restrict the basal levels of pro-inflammatory cytokines secreted by circulating PBMCs in epileptic patients. Upon TLR stimulation however, there was not such a differential trend between PBMCs purified from placebo- and formulation 7E-treated subjects. Given the mild inhibition observed for the basal cytokine secretion in unstimulated PBMCs, it is likely that potent LPS or Pam2CSK4 stimulation may overcome the restrictive effect of formulation 7E on TLR-induced secretion of pro-inflammatory cytokines.

### Correlation Between Changes in Seizures Frequency and T-Cell Cytokines

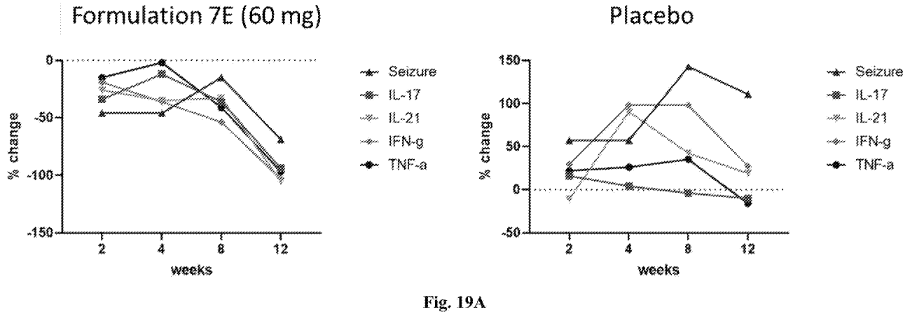

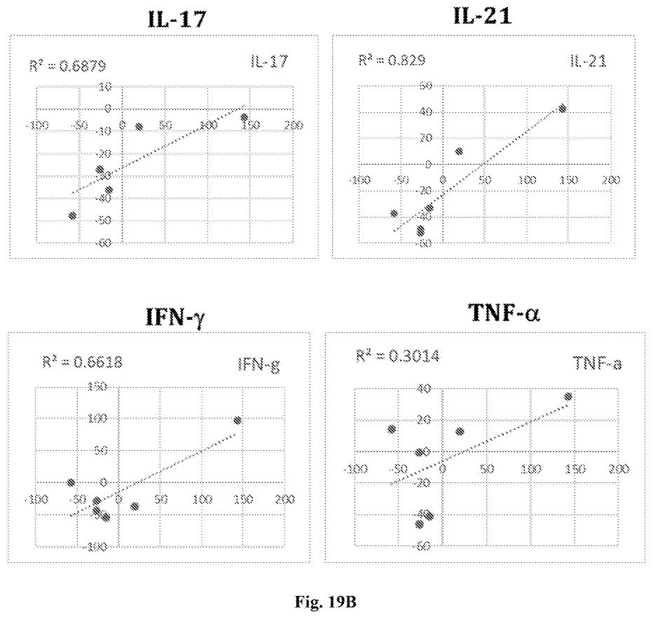

As shown in FIG. 19, changes in seizures frequency correlate with a decrease in T-cell cytokine secretion in individual patients.

Overall, this data further demonstrates that epileptic patients have dysregulated pro-inflammatory cells at peripheral/systemic level which might contribute to amplifying and sustaining neuronal damage.

### Pharmacodynamic of Foxp3+ Cells in Peripheral Mononuclear Cells (PBMCs)

In vitro, the treatment of human CD4⁺ T cells activated under Th17 skewing conditions with ivermectin leads to the up-regulation of FoxP3 mRNA levels followed by 50 % increase in the percentage of Foxp3⁺ T cells during the co-culture (see Example 9). Therefore, the percentage of Foxp3⁺ cells was evaluated in PBMCs purified from patients with epilepsy after oral administrations of 7E gel capsules for 12 weeks.

### Methods

Whole peripheral blood samples from clinical trial subjects (6 treated and 2 placebo subjects per cohort) were collected at Day 0 before dosing begins (pre-dose/baseline) as well as after 2, 4, 8, and 12 weeks of treatment with 20 mg, 40 mg, or 60 mg of 7E. PBMCs were purified by Ficoll-Paque and cryopreserved until ex vivo analysis. On the day of the experiment, cells from each sample were thawed and allowed to rest overnight in complete tissue culture media in a 37° C., 5% CO₂ incubator. Then, cells were counted and stained with a viability dye and anti-CD4 antibody. After washing the cells were fixed, permeabilized, and then stained for FOXP3. Samples were analyzed on a Luminex Guava easyCyte 11HT 9-color flow cytometer using appropriate single-color controls and compensation. Flow cytometry files were analyzed using FlowJo version 10.8.1.

### Results

The intracellular Flow Cytometry analysis of PBMCs purified from patients with epilepsy participating in Phase 2 clinical study (EQU-201) determined the percentage of Foxp3⁺/CD4⁺ cells ranging between 0.18%-17.1% (Table 18). Oral administration of 60 mg of 7E significantly decreased the percentage of Foxp3⁺/CD⁴+ cells over the 12 weeks of treatment compared to pre-dose levels (FIG. 20A). Four out five samples purified from patients with epilepsy treated with 7E at 40 mg (Cohort 3) demonstrated an increased in the percentage of Foxp3⁺/CD4⁺ cells compared to the pre-dose levels, with statistical significance achieved after 12 weeks of treatment (FIG. 20B).

TABLE 18
Intracellular staining to determine the percentage of
Foxp3⁺/CD4⁺ in PBMCs from patients with epilepsy on
Day 1 before dosing begins (pre-dose) and after 2, 4, 8,
and 12 weeks of 7E treatment (60 mg, 40 mg or 20 mg) or placebo
(marked by *). The numbers in Italic font indicate samples
were processed in a different facility from rest of the samples.

| | | | | | |
|---|---|---|---|---|---|
| Patient | Pre-dose | 2 weeks | 4 weeks | 8 weeks | 12 weeks |

Cohort 4 (60 mg)

| | | | | | |
|---|---|---|---|---|---|
| 17-0005 | 11.8 | 7.11 | 7.01 | 4.23 | 2.26 |
| 17-0006* | 2.64 | 4.81 | 3.35 | 6.07 | 4.47 |
| 21-0001 | 7.54 | 8.77 | 6.56 | 6.6 | |
| 22-0004 | 10.6 | 6.02 | 5.83 | 6.66 | 5.62 |
| 22-0005 | 9.94 | 5.15 | 5.57 | 5.1 | 4.36 |
| 22-0006* | 0.27 | 0.18 | 0.19 | 0.18 | |
| 14-0003 | 4.37 | 5.91 | 4.91 | 4.11 | 1.84 |

Cohort 3 (40 mg)

| | | | | | |
|---|---|---|---|---|---|
| 14-0001 | 2.57 | 5.46 | 2.33 | 2.08 | 5.21 |
| 14-0002 | 1.56 | 1.64 | 2.04 | 4.13 | 8.28 |
| 17-0001 | 7.49 | 7.56 | 4.01 | 17.1 | 7.24 |
| 17-0002 | 8.98 | 16.8 | 8.48 | 9.26 | 11.8 |
| 17-0003 | 1.45 | 6.24 | 15.4 | 7.32 | 6.66 |

Cohort 2 (20 mg)

| | | | | | |
|---|---|---|---|---|---|
| 22-0001 | 1.57 | 3.96 | 2.43 | 2.03 | 5.76 |
| 22-0002 | 2.54 | 3.96 | 1.72 | 1.9 | 8.51 |
| 22-0003 | | | | 5.34 | 4.65 |
| 22-0001 | | | 9.15 | 8.4 | 9.65 |

### Example 17—Patient Dosed Phase 2b Study (Daily. 16 Weeks) (Prophetic Example)

This clinical trial is a randomized Phase 2b study of adjunctive formulation 7E for uncontrolled focal onset seizures.

### Objectives

The primary objective is to evaluate the efficacy of ivermectin (formulation 7E) in doses of 20 mg and 60 mg per day as adjunctive therapy compared with placebo for focal onset seizures in subjects with epilepsy.

Secondary objectives include: (1) To assess the efficacy of formulation 7E at doses of 20 mg and 60 mg during a maintenance phase; (2) to assess the efficacy of formulation 7E at doses of 20 mg and 60 mg in specified seizure types; (3) to assess the subject's impression of change at doses of 20 mg and 60 mg; (4) and to assess the effect on quality of life of formulation 7E in subjects with focal onset seizures at doses of 20 mg and 60 mg daily.

Safety objectives include to assess the safety and tolerability of formulation 7E in doses of 20 mg and 60 mg per day in subjects with focal onset seizures.

Exploratory objectives include (1) to assess blood plasma exposure of formulation 7E at daily doses of 20 mg and 60 mg in subjects with focal onset seizures; assessed also as a function of concomitant anti-seizure medications (ASM)s and as a function of Child-Turcotte-Pugh (CTP) class of hepatic disease (A and B, class C is excluded)), and serum levels of albumin, bilirubin and PT, and (2) to assess the plasma levels of exploratory biomarkers associated with epilepsy.

### Endpoints

Primary efficacy endpoints include the median percentage change in the overall number of countable observable seizures per 28-day period relative to baseline in each treatment arm during the double-blind treatment period compared with placebo.

Secondary efficacy endpoints include (1) median percentage change in the overall number of countable observable seizures per 28-day period relative to baseline in each treatment arm during the maintenance phase (treatment weeks 5 through 16) compared with placebo; (2) ≥50% responder rates in the treated arms as compared with placebo during double blind components of the study, which consist of the medication activation and maintenance period; (3) ≥50% responder rates in the treated arms as compared with placebo during the maintenance period alone (treatment weeks 5-16); (4) difference in Patient Global Impression of Change Scale Score between each treated cohort and placebo at days 56 and 112; (5) median percentage change in the number of countable observable seizures by subtype (focal aware with motor component, focal impaired aware, and focal to bilateral tonic-clonic) per 28 days during the maintenance period (treatment weeks 5-16) and during the entire double blind period in the treated and placebo arms; (6) percent of subjects who are seizure free by study days 29-112 (treatment weeks 5-16); (7) percent of subjects who are seizure free in treated arms as compared with placebo during treatment weeks 5-16; (8) ≥70% and ≥90% response rates in treated arms compared with placebo during the maintenance period (treatment weeks 5-16); and (9) change from visit 2 (enrollment) in the Patient weighted Quality of Life in Epilepsy (QOLIE 31-P) scale score at days 56 and 112 in each treated arm as compared with placebo.

Safety and tolerability endpoints include (1) number of subjects who withdraw from treatment because of an AE in each treatment arm; (2) number of adverse events (CTCAE grade 2 or higher) in the treatment arms compared with placebo; and (3) change from visit 2 in Columbia-Suicide Severity Rating Scale responses in each treated arm as compared with placebo at each measured timepoint.

PK and PD analyses endpoints include (1) blood plasma levels of formulation 7E over time for each cohort; (2) effect of concomitant medications on plasma levels of formulation 7E; (3) correlation between CTP class (A and B, class C is excluded) and plasma levels of formulation 7E in each cohort; (4) correlation of levels of serum albumin, serum bilirubin and prothrombin time (PT) with formulation 7E plasma levels in each cohort; (5) correlation between formulation 7E levels and efficacy; and (6) plasma level evaluation of exploratory biomarkers associated with epilepsy.

### Number of Subjects

Approximately 300 subjects, 100 per arm.

### Population

Participants 18-65 years of age at time of consent who have been diagnosed with focal epilepsy according to International League Against Epilepsy (ILAE) Classification of the Epilepsies 2017 criteria (ILAE 2017) and who are uncontrolled on one to three concomitant anti-seizure medications (ASMs) at optimal stable dosages for ≥4 weeks prior to screening and throughout the treatment period.

### Investigational Product, Dosage and Mode of Administration

Formulation 7E is supplied as 20 mg gel capsules. Placebo is supplied as gel capsules, matching the formulation 7E product, but with no active ingredient Dosing: Placebo, formulation 7E 20 mg, or formulation 7E 60 mg administered orally, once daily. There are three pills per dose (three placebos, 1 20 mg pill and 2 placebo pills, or 3 20 mg pills).

### Duration of Treatment

16 weeks with the option for 12 month open-label extension (OLE) for all subjects.

### Study Duration

Per subject: 8 weeks or longer baseline plus 16 weeks treatment for each patient; option for 12 month OLE. Per study: The last patient's last visit defines the end of the 202 double-blind study. The total duration is expected to be approximately 24 months, plus the 12 month OLE.

### Eligibility

Inclusion criteria include (1) age 18-65 years at time of informed consent; (2) diagnosed with focal epilepsy according to ILAE (2017) criteria. Diagnosis to include clinical history and an EEG consistent with focal epilepsy. A normal interictal EEG is allowed when the clinical history is consistent with focal epilepsy; (3) subject has no seizures that are not focal by the ILAE 2017 criteria; (4) subject must have 8 countable, observable focal seizures during the 8-week baseline period prior to randomization, including at least 3 in each 4-week period with no 21-day seizure-free period. These seizures must be observable (focal aware with motor component, focal impaired awareness, focal to bilateral tonic-clonic) and as such may not include focal aware seizures without a detectable motor component, aphasia, or other observable symptom; (5) must have had a brain MRI or contrast-enhanced head CT scan with an available report (images need not be available) that has been performed within the past 10 years (but not prior to the subject's diagnostic assessment for epilepsy) and that is negative for confounding conditions such as tumor, infection, demyelinating disease, or other progressive neurological disease. Remote stroke that may represent the etiology for epilepsy is allowed. If no such CT or MRI report is available, a potential subject will be asked to undergo a head CT scan with intravenous contrast to meet eligibility criteria prior to study enrollment; (6) seizures uncontrolled after an adequate trial of at least 1 ASM within the last 2 years; (7) currently receiving treatment with 1-3 ASMs with doses stable for at least 4 weeks prior to screening. These medications must stay stable during the 8-week baseline period and during the 16-week treatment period. In the case that the plasma level of a concomitant ASM changes, the subject and their physician may then modify the dose to maintain the plasma level that was present prior to beginning the study drug. and must document the change in the EDC system.

Exclusion criteria include (1) history of hypersensitivity to ivermectin or to any of the excipients in the formulation 7E gel capsule; (2) history of status epilepticus in the past 1 year from screening; (3) history of pseudo- or nonepileptic seizures, or other nonepileptic events that could be confused with epileptic seizures, within the past 5 years; (4) history of traumatic brain injury within 30 days prior to screening; (5) respective epilepsy surgery within 1 year; epilepsy-related radiosurgery within 2 years or Ventriculoperitoneal shunt placement within 1 year; (7) presence of progressive neurological disorder or other progressive disorder or unstable medical condition(s) that may confound study results. History of long QT syndrome, family history of sudden death of unknown cause.

### Overall Study Design and Plan

This study will be a Phase 2 multinational, double-blind, placebo-controlled, randomized (1:1:1), efficacy and safety study of adjunctive formulation 7E for the treatment of focal onset seizures in subjects aged 18 to 65 years, who have been diagnosed with epilepsy according to International League Against Epilepsy (ILAE) Classification of the Epilepsies 2017 criteria (ILAE 2017). Subjects' seizures must be uncontrolled on one to three concomitant ASMs at stable dosages for ≥4 weeks prior to screening.

Up to 300 eligible subjects will be sequentially, stratified by country and then randomized at a 1:1:1 ratio to receive 20 mg of formulation 7E, 60 mg of formulation 7E or a matched placebo (see Table 19). Before dosing, all subjects will undergo a screening assessment, including seizure identification and diagnostic review, measurement of vital signs, physical and neurological examinations, ECG, safety labs (AST, ALT ALP, total bilirubin, CBC with differential, blood chemistry, PT/PTT/INR, albumin, total protein, and urinalysis), urine toxicology screen, pregnancy testing for premenopausal females, and completion of the C-SSRS. Screening will be followed by an 8-week or longer prospective baseline assessment period to determine seizure frequency.

TABLE 19
Dose Cohorts, Planned

| No. of Subjects | Doses |
|---|---|
| 100 | Formulation 7E 60 mg (3 × 20 mg gelcaps) |
| 100 | Formulation 7E 20 mg (1 × 20 mg pill + |
| | 2 × matching placebo gelcaps) |
| 100 | Formulation 7E 0 mg (3 × matching |
| | placebo gelcaps) |

The treatment portion of the study will be comprised of a 4-week double-blind medication activation period and a 12-week double-blind maintenance period. Subjects will continue with dosing through the 16 weeks if they do not meet stopping/withdrawal criteria or decide to withdraw from the study. The primary study endpoint will be the median percentage change in seizure frequency per four weeks during the 16-week double-blind treatment period between each active treatment arm relative to placebo.

There will be an interim analysis for safety and statistical power reassessment. When 50% of the sample has received study drug for at least 16 weeks, an independent non-blinded statistician will review the data and assess the statistical power of the study. The statistician will advise the Sponsor on the need for sample size readjustments to ensure the statistical power of 90% is maintained. There will be no efficacy assessment at the interim analysis.

Open-Label Extension (OLE): Once the 16-week study dosing period is complete, all subjects who continue to meet eligibility criteria may enroll in a 12 month OLE. All subjects will provide separate informed consent prior to entering the OLE and the OLE will be conducted according to the principles set forth by the International Council on Harmonisation (ICH) for Good Clinical Practice (GCP) and the Declaration of Helsinki. The initial data cut will be 12 months from initiation of the OLE.

The dual aims of this OLE are to provide continued access to study drug for subjects who would like such access/are benefitting from the Investigational Medicinal Product (IMP) and to gather long-term safety and efficacy data. The recommended starting OLE dose is 60 mg. Lower starting doses may be chosen at the discretion of the Principal Investigator (PI) (20 mg or 40 mg). Subjects approved by the sponsor to enter the OLE prior to completion of the 16-week double blind portion of the study (as a mechanism to down dose), will start at 20 mg daily, with the option to increase the dose if tolerated and as needed. During the OLE, dose adjustments to formulation 7E are allowed (20 mg, 40 mg, and 60 mg doses are available) as are adjustments to other ASMs including additions and discontinuations, provided the inclusion criteria of 1-3 concomitant ASMs continues to be met. Subjects will have in-person visits every 13 weeks and telephone interviews every 13 weeks, for the first six months, so that there is follow-up every 6.5 weeks. After the first six months, subjects will have in-person visits every 13 weeks for up to 12 months from OLE entry. Subject safety, tolerability, and efficacy data will be collected as in the SOA. The objectives of the OLE are to assess the long-term safety, tolerability, and efficacy of formulation 7E. Efficacy outcomes include the median percent change in focal seizure frequency at consecutive six-month intervals as compared with baseline and ≥50%, ≥7 0%, ≥90% and 100% responder rates at 12-months overall and by dose. Tolerability will be reported descriptively as the percent of subjects who discontinue treatment during the first 3 months, and then at 6 and 12 months. Dose changes, duration and median daily dose will be reported descriptively at 3 months, and then at 6 and 12 months. Safety assessments include frequency and severity of TEAEs and C-SSRS responses and will be reported descriptively, per dose, including those that lead to discontinuation, at 3 months, 6 months, and 12 months.

Follow-Up: Follow-up visits will occur 30 days (±4 days) after the last dose of study drug for subjects who choose not to continue into the OLE and for subjects when they choose to end participation in the OLE, up to 13 months after OLE entry. At follow-up visits, subjects will undergo safety testing and assessments. If another therapy is started within 30 days after the last dose of study drug, the Follow-up Visit should be conducted before the start of the other therapy.

### Example 18—Use of Ivermectin in a Model for Multiple Sclerosis (MS)

Multiple Sclerosis (MS) is immune-mediated demyelination disease of the central nervous system, which leads to progressive autonomic, visual, and motor dysfunction. MS is characterized by a two-stage disability progression: Relapsing-remitting MS (RRMS), characterized by inflammation, and secondary progressive MS (SPMS) (characterized by neurodegeneration).

Accordingly, the effect of ivermectin, administered therapeutically, on experimental autoimmune encephalomyelitis (EAE) development in C₅₇BL/6 mice was examined. EAE is the most commonly used mouse model of MS. Because of its many similarities to MS, EAE is used to study pathogenesis of autoimmunity, CNS inflammation, demyelination, cell trafficking, and tolerance induction. EAE is characterized by paralysis, CNS inflammation, and demyelination. EAE is mediated by myelin-specific CD4⁺ T cells, but CDS⁺ cells and B cells may also play a role in some models of EAE. EAE is induced in C57BL/6 mice by immunization with MOG₃₅₋₅₅ or MOG1-12s in CFA emulsion followed by administration of pertussis toxin (PTX) in PBS. The emulsion provides antigen that initiates expansion and differentiation of MOG-specific autoimmune T cells. PTX enhances EAE development by providing additional adjuvant and facilitating entrance of autoimmune T cells into the CNS. Fingolimod (FTY720, Gilenya) is the most commonly used positive control in this model. When mice are followed for a longer period of time (longer than 6 weeks), disease usually slowly increases in severity, resembling the chronic progressive course of disease observed in human MS patients.

### Methods

Vehicle was 1.6% DMSO in water. Ivermectin, provided as powder, was formulated at the start of the study in DMSO at 50 mg/mL, and aliquots for 3 to 4 days of dosing were prepared. These were frozen at −20° C. Every 3 to 4 days, one aliquot was thawed and diluted with water to create a fine suspension at 0.8 mg/mL (for 4 mg/kg dosing). Final DMSO concentration was 1.6%. The mid and low doses of ivermectin were formulated by diluting the high dose with vehicle. Per customer, all dosing formulations were stable for up to 4 days at 4° C. FTY720 stock solution (30 mg/mL in ethanol) was prepared for all ongoing studies. Daily, the FTY720 stock solution was diluted with water to the final dosing concentration of 0.6 mg/mL.

A balanced distribution of mice was confirmed by comparing mean day of onset, average score at EAE onset, average score on Day 14 and average weight on Day 13.

There were five groups in the study, with 12 mice in each group (administration oral, once daily): (1) Vehicle (negative control), (2) 7E, 1 mg/kg, (3) (2) 7E, 2 mg/kg, (4) 7E, 4 mg/kg, and (5) FTY720, 3 mg/kg (positive control). EAE was induced on Day 0 by myelin oligodendrocyte glycoprotein (MOG₃₅₋₅₅)/complete Freund's adjuvant (CFA) immunization, followed by pertussis toxin (PTX) injections on Days O and 1. Treatment was therapeutic, starting on Day 14. At the time treatment started, all but one mouse in the study had clinical signs of EAE for up to 7 days. Treatment continued through Day 34. Mice were observed through the end of the study, Day 35.

Clinical scores were assessed as follows: 1=limp tail, 2=partial hind leg paralysis, 3=complete hind leg paralysis, 4=complete hindleg and partial front leg paralysis; 5=moribund. For each spine, one hematoxylin and eosin (H&E)-stained slide and one anti-myelin basic protein (MBP)-stained slide was prepared and analyzed for inflammation and demyelination, respectively.

### Results

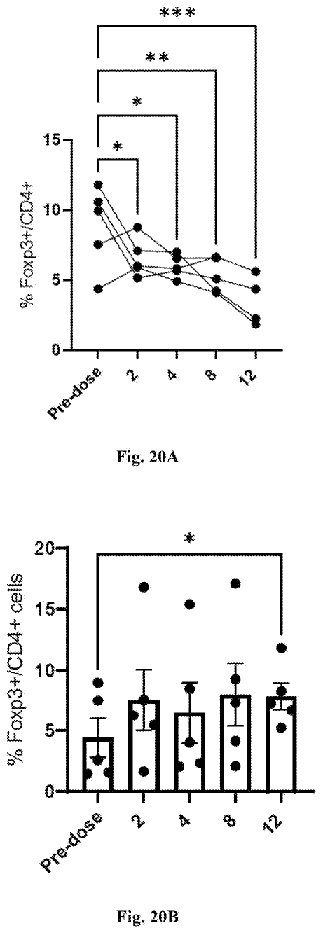

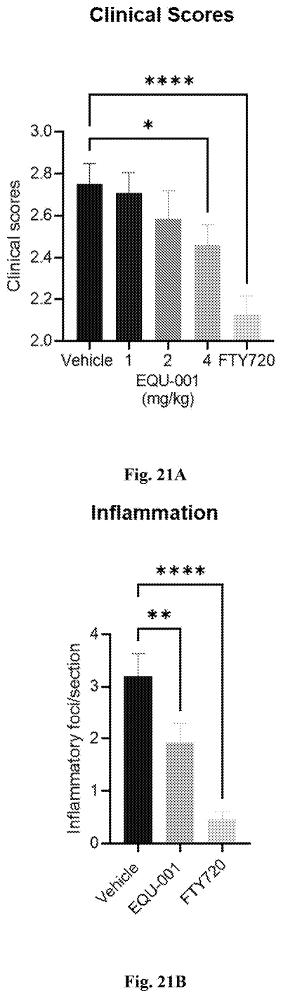

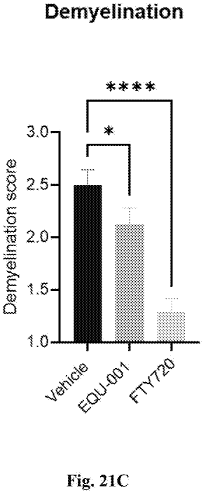

As shown in FIG. 21, ivermectin significantly downregulates limb paralysis in an EAE model of MS.

### Example 19—Use of Ivermectin in a Traumatic Brain Injury (TBI) Model

The objective of this study was to investigate the efficacy of ivermectin after Traumatic Brain Injury (TBI) in mice by behavioral readouts and MRI.

### Methods

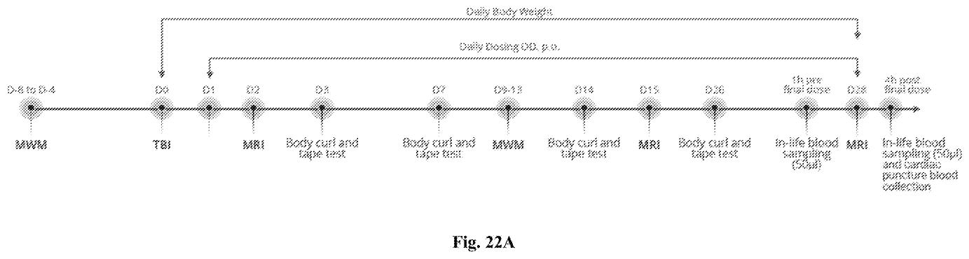

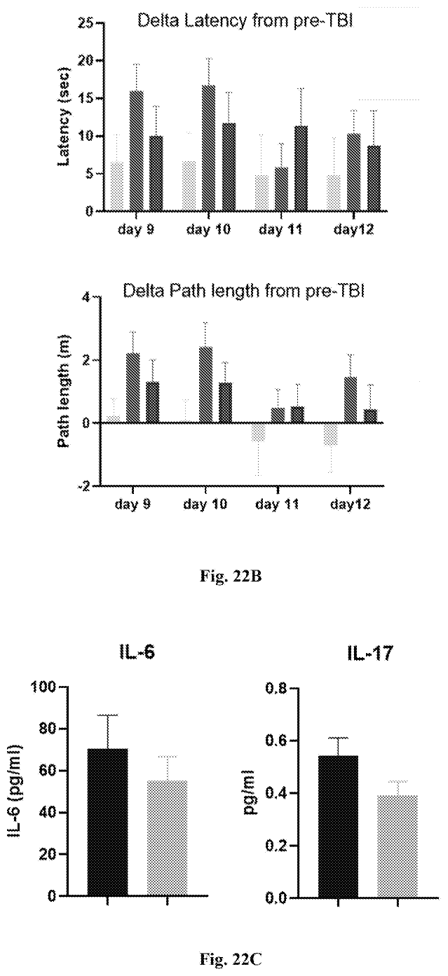

A pneumatic impact device was used to deliver precise and controlled cortical contusions to an animal. Multiple behavioral readouts and MRI were performed during the 28 days follow-up period. Test compound administration was done daily via oral administration during the 28 days follow-up period. Adhesive tape removal test and elevated body swing test were performed at 4 different timepoints (3-, 7-, 14- and 26-days post TBI). A Morris Water Maze Test was performed twice, once before TBI and once after TBI. Pre-training was done during a 5-day period (8-4 days pre TBI) and post TBI 9-13 days after injury. Magnetic resonance imaging (MRI) was performed at 3 different timepoints, 2-, 15-, and 28-days post injury. At the endpoint, day 28 plasma samples were collected pre-final dose and 4-hours post final dose. After the last in-life blood collection brains were collected and carefully hemisected in 2 parts. Ivermectin was dissolved in water. The experimental setup is shown in FIG. 22A.

Animals were grouped as follows: Group 1: Sham operated mice treated with vehicle (n=11); Group 2: TBI (2.5 mm, 3 m/s) operated mice treated with vehicle (n=14); Group 3: TBI operated mice treated with ivermectin at 3 mg/kg once a day orally (n=15). The Morris Water Maze (MWM) was used to assess learning/memory function.

Morris Water Maze test: pre-training (5 days) and post TBI day 9-13 (prob trial). Mice were brought to the experimental room for at least 30 min acclimation to the experimental room conditions prior to testing. Water maze task was originally designed by Morris et al. (J Neurosci Methods. 1984; 11: 47-60). Acquisition trials were performed at pre-testing (baseline) and post-TBI to determine the mouse's ability to learn the spatial relationship between distant cues and the hidden escape platform, which remain in the same location for all place trials. Testing was performed in a large tank (120 cm in diameter) filled with water at a temperature of 24.0±1.0° C. A submerged platform (square platform: 14×14 cm; 1.5 cm below water surface) is placed in the middle of the NE quadrant. The starting locations, which were labeled N, NE, E, SE, S, SW, W, NW, are located arbitrarily on the pool rim. The mice was placed into the pool with their nose pointing toward the wall at one of the starting points. Platform was on same location during pre-testing and post-TBI testing.

### Results

As shown in FIG. 22B, ivermectin reduces the memory and learning deficits induced by TBI. Treatment with ivermectin also reduced expression of inflammatory cytokines in mice with TBI (FIG. 22C).

### Example 20—Use of Ivermectin for the Treatment for Infantile Spasms in a Validated Animal Model of Infantile Spasms

### Methods

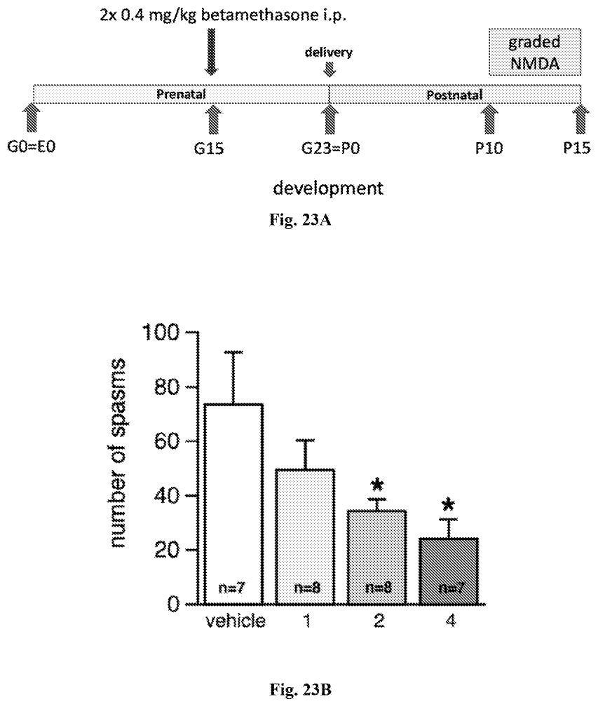

A two-stage model of infantile spasms was developed (FIG. 23A). Pregnant Sprague-Dawley rats receive two injections of betamethasone on gestational (G) day 15. Offspring of those pregnancies are used for experiments during infancy, postnatal days (P) 10-15. Spasms are induced by i.p. injection of N-methyl-D-aspartic acid (NMDA), which is graded depending on the postnatal age of the subjects.

Treatment groups were as follows: Three initial doses were of 1, 2 or 4 mg/kg ivermectin delivered i.p. in a single bolus dose at 2 μm daily dissolved in vehicle in concentration of 1, 2 or 4 mg/5 ml of vehicle. Days of dosing were P10, P11, P12, P13, P14 and P15. Vehicle was used in the same volumes as the volume used for ivermectin (5 ml/kg for i.p. delivery). Ivermectin was dissolved in beta-cyclodextrin (0.5% solution).

### Results

Both 2 and 4 mg/kg ivermectin treatments significantly suppressed occurrence of spasms in comparison to controls (injected with vehicle) (FIG. 23B).

We claim:

1. A method of treating or preventing an inflammatory disorder, the method comprising administering to a subject in need thereof a pharmaceutical composition comprising:
(i) about 1% to about 15% of a compound, wherein the compound is a compound of Formula I: 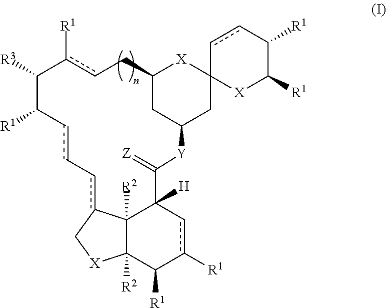
or a pharmaceutically acceptable salt, stereoisomer, polymorph, solvate, or isotopically labeled compound thereof, wherein
each occurrence of X is independently selected from —CH₂—, —NH—, —O—, —S—, —SO— and —SO₂—;
Y is selected from —CH₂—, —O—, —NH—, and —S—;
Z is selected from O and S;
each occurrence of [symbol] is a single bond or a double bond;
n is an integer 0-6; and
each occurrence of R¹ is independently selected from halogen, —R, —OR, —NO₂, —NCS, —CN, —CF₃, —OCF₃, —NHR, —N(R)₂, —OC(O)R, —C(O)OR, —SR, —C(O)R, —C(O)C(O)R, —C(O)CH₂C(O)R, —C(S)R, —C(S)OR, —C(O)C(O)OR, —C(O)C(O)N(R)₂, —C(O)N(R)₂, —OC(O)N(R)₂, —C(S)N(R)₂, —N(R)C(O)OR, —N(R)C(O)R, —N(R)C(S)R, —N(R)C(O)N(R)₂, —N(R)C(S)N(R)₂, —N(COR)COR, —N(OR)R, —C(═NH)N(R)₂, —C(O)N(OR)R, —C(═NOR)R, —OP(O)(OR)₂, —P(O)(R)₂, —P(O)(OR)₂, —P(O)(H)(OR), —CH₂—OR, and —CH₂—O—CH₂—R;
each occurrence of R² is independently selected from independently selected from H, OH, O—C₁₋₄alkyl, —OC(O)C₁₋₄alkyl, —OC(O)NH₂, and —OC(O)NHC₁₋₄alkyl;
each occurrence of R³ is mono, di, or triglycoside, or —OC(O)—(C₃-C₅)alkenyl;
each R is independently selected from H, —(C₁-C₁₂)alkyl, —(C₃-C₁₀)-cycloalkyl, —(C₃-C₁₀)-cycloalkenyl, —[(C₃-C₁₀)cycloalkyl]-(C₁-C₁₂)alkyl, —[(C₃-C₁₀)cycloalkenyl]-(C₁-C₁₂)alkyl, —[(C₃-C₁₀)cycloalkenyl]-(C₁-C₁₂)alkyl, —[(C₃-C₁₀)cycloalkyl]—O—(C₁-C₁₂)alkyl, —[(C₃-C₁₀)cycloalkenyl]—O—(C₁-C₁₂)alkyl, —(C₆-C₁₀)aryl, (C₆-C₁₀)aryl-(C₁-C₁₂)alkyl, —(C₆-C₁₀)aryl-O—(C₁-C₁₂)alkyl, (C₆-C₁₀)aryl-N(R″)—(C₁-C₁₂)alkyl, 3- to 10-membered heterocyclyl, (3- to 10-membered heterocyclyl)-(C₁-C₁₂)alkyl, (3- to 10-membered heterocyclyl)-O—(C₁-C₁₂)alkyl, (3- to 10-membered heterocyclyl)-N(R″)—(C₁-C₁₂)alkyl, 5- to 10-membered heteroaryl, (5- to 10-membered heteroaryl)-(C₁-C₁₂)-alkyl, (5- to 10-membered heteroaryl)-O—(C₁-C₁₂)-alkyl and (5- to 10-membered heteroaryl)-N(R″)—(C₁-C₁₂)-alkyl;
each heterocyclyl has 1-4 heteroatoms independently selected from N, NH, O, S, SO, and SO₂, and
heteroaryl has 1-4 heteroatoms independently selected from N, NH, O, and S;
each occurrence of R is independently unsubstituted or is substituted with 1 to 5 R′;
each occurrence of R′ is halo, OH, oxo, —CH₂OR″, —CH₂N(R″)₂, —C(O)N(R″)₂, —C(O)OR″, —NO₂, —NCS, —CN, —CF₃, —OCF₃ and —N(R″)₂; and
each occurrence of R″ is independently H, C₁₋₆ alkyl, C₂₋₆ alkenyl, C₃₋₆ cycloalkyl, C₃₋₆ cycloalkenyl, 3- to 6-membered heterocyclyl, 5- to 10-membered heteroaryl, and (C₆-C₁₀)-aryl;
(ii) about 20% to about 40% of a first surfactant comprising one or more of:
(a) mono-, di-, and/or tri-fatty acid esters of glycerol;
(b) mono- and/or di-fatty acid esters of 1,2-propylene glycol; and
(c) mono- and/or di-fatty acid esters of polyethylene glycol wherein the fatty acids are selected from C₆ to C₁₀ fatty acids; and
(iii) about 15% to about 70% of a second surfactant selected from one or more of a polysorbate surfactant and/or a fatty acid ester of sorbitan.

2. The method of claim 1, wherein the compound is a compound of Formula II: 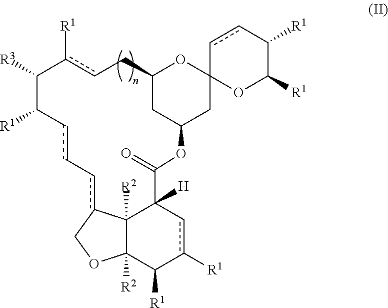
or a pharmaceutically acceptable salt, stereoisomer, polymorph, solvate or isotopically labeled compound thereof, wherein n, R¹, R², and R³ are each as defined in Formula I.

3. The method of claim 1 or 2, wherein the compound is a compound of Formula III: 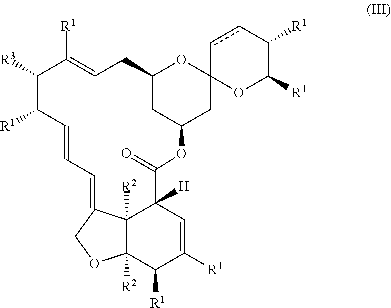
or a pharmaceutically acceptable salt, stereoisomer, polymorph, solvate or isotopically labeled compound thereof, wherein R¹, R², and R³ are each as defined in Formula I.

4. The method of any one of the preceding claims, wherein the compound is a compound of Formula IV: 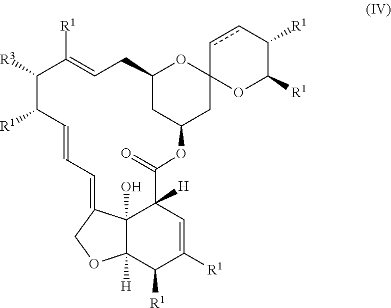
or a pharmaceutically acceptable salt, stereoisomer, polymorph, solvate or isotopically labeled compound thereof, wherein
each occurrence of R¹ is independently selected from halogen, —R, —OR, —NO₂, —NCS, —CN, —CF₃, —OCF₃, —NHR, —N(R)₂, —OC(O)R, —C(O)OR, —C(O)N(R)₂, —OC(O)N(R)₂, —N(R)C(O)OR, —N(R)C(O)R, —N(R)C(O)N(R)₂, —CH₂—OR, and —CH₂—O—CH₂—R;
each occurrence of R³ is mono, di, or triglycoside, or OC(O)—(C₃-C₅) alkenyl; and R, R′ and R″ are each as defined in Formula I.

5. The method of any one of the preceding claims, wherein the compound is a compound of Formula V: 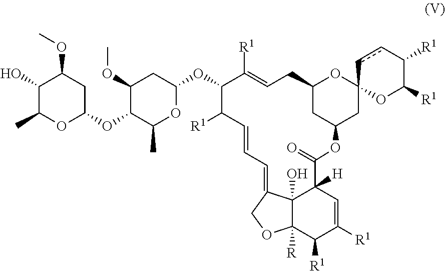
or a pharmaceutically acceptable salt, stereoisomer, polymorph, solvate or isotopically labeled compound thereof, wherein
each occurrence of R¹ is independently selected from halogen, —R, —OR, —NO₂, —NCS, —CN, —CF₃, —OCF₃, —NHR, —N(R)₂, —OC(O)R, —C(O)OR, —C(O)N(R)₂, —OC(O)N(R)₂, —N(R)C(O)OR, —N(R)C(O)R, —N(R)C(O)N(R)₂, —CH₂—OR, and —CH₂—O—CH₂—R; and
R, R′ and R″ are each as defined in Formula I.

6. The method of any one of the preceding claims, wherein the pharmaceutical composition comprises about 1% to about 15% ivermectin comprising a compound of Formula VI and a compound of Formula VII:

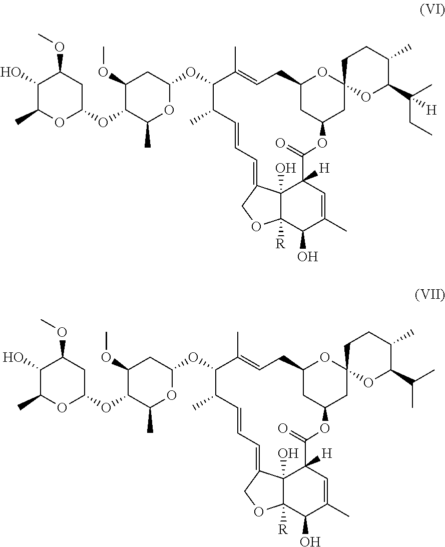

7. The method of claim 6, wherein the ivermectin comprises at least about 70% of a compound of Formula VI and less than about 30% of a compound of Formula VII.

8. The method of claim 7, wherein the ivermectin comprises at least about 90% of a compound of Formula VI and less than about 10% of a compound of Formula VII.

9. The method of any one of the preceding claims, wherein the pharmaceutical composition comprises about 3% to about 12% of ivermectin.

10. The method of claim 9, wherein the pharmaceutical composition comprises about 5% to about 10% of a ivermectin.

11. The method of any one of claims 1 to 10, wherein the fatty acids for the first surfactant are selected from C₈ to C₁₀ fatty acids.

12. The method of claim 11, wherein the first surfactant comprises mono- and di-fatty acid esters of glycerol.

13. The method of any one of claims 1 to 12, wherein the first surfactant is selected from Masester M8120, Capryol 90, Labrasol ALF, and combinations thereof.

14. The method of any one of claims 1 to 13, wherein the second surfactant is selected from polysorbate 80 (Tween 80), sorbitan monolaurate (Span 20), and combinations thereof.

15. The method of any one of claims 1 to 14 comprising about 25% to about 30% of the first surfactant.

16. The method of any one of claims 1 to 15 comprising about 15% to about 20% of the second surfactant.

17. The method of any one of claims 1 to 15 comprising about 30% to about 35% of the second surfactant.

18. The method of any one of claims 1 to 15 comprising about 60% to about 65% of the second surfactant.

19. The method of any one of claims 1 to 17, further comprising about 5% to about 55% vitamin E TPGS; or about 30% to about 50% vitamin E TPGS.

20. The method of any one of claims 1 to 10 comprising: (i) about 5% to about 10% ivermectin; (ii) about 25% to about 30% mono- and di-C₈ to C₁₀ fatty acid esters of glycerol; (iii) about 15% to about 30% of a polysorbate surfactant; and (iv) about 30% to about 50% vitamin E TPGS.

21. The method of any one of claims 1 to 10 comprising: (i) about 8% ivermectin; (ii) about 27.6% mono- and di-C₈ to C₁₀ fatty acid esters of glycerol; (iii) about 32.2% polysorbate 80; and (iv) about 32.2% vitamin E TPGS.

22. The method of any one of claims 1 to 10 comprising: (i) about 8% ivermectin; (ii) about 27.6% Masester E8120; (iii) about 32.2% polysorbate 80; and (iv) about 32.2% vitamin E TPGS.

23. The method according to any one of claims 1 to 10 comprising: (i) about 8% ivermectin; (ii) about 27.6% mono- and di-C₈ to C₁₀ fatty acid esters of glycerol; (iii) about 18.4% polysorbate 80; and (iv) about 46% vitamin E TPGS.

24. The method of any one of claims 1 to 10 comprising: (i) about 8% ivermectin; (ii) about 27.6% Masester E8120; (iii) about 18.4% polysorbate 80; and (iv) about 46% vitamin E TPGS.

25. The method of any one of claims 1 to 24, wherein the pharmaceutical composition is administered to the subject in a pharmaceutical dosage form comprising the composition in a gelatin capsule.

26. A method of treating or preventing an inflammatory disorder comprising administering to a subject in need thereof a pharmaceutical composition of any one of claims 1 to 25.

27. The method of claim 26, wherein the inflammatory disorder is an autoimmune disorder.

28. The method of claim 27, wherein the autoimmune disorder is limbic encephalitis, rheumatoid arthritis (RA), systemic lupus erythematosus (SLE), Psoriasis, Crohn's disease or graft-versus-host disease (GVHD).

29. The method of claim 26, wherein the inflammatory disorder is a neuroinflammatory disorder.

30. The method of claim 29, wherein the neuroinflammatory disorder is Alzheimer's disease (AD), Parkinson's disease (PD), stroke, seizure disorders, epilepsy, infantile spasms, or amyotrophic lateral sclerosis (ALS).

31. The method of any one of claims 26-30, wherein the subject is a mammal.

32. The method of claim 31, wherein the subject is a human.

33. The method of any one of claims 1-32, wherein about 10 mg to about 120 mg of the compound is administered to the subject.

34. The method of claim 33, wherein about 10 mg to about 80 mg of the compound is administered to the subject.

35. The method of claim 33, wherein about 20 mg to about 40 mg of the compound is administered to the subject.

36. The method of claim 33, wherein about 10 mg, about 20 mg, about 40 mg, about 60 mg, about 80 mg, or about 120 mg of the compound is administered to the subject.

37. The method of claim 36, wherein about 20 mg of the compound is administered to the subject.

38. The method of claim 36, wherein about 60 mg of the compound is administered to the subject.

39. The method of any one of the claims 1-38, wherein the pharmaceutical composition is administered once a day, every other day, or every three days.

40. The method of claim 39, wherein the pharmaceutical composition is administered once a day.

41. The method of any one of claims 1-40, wherein the pharmaceutical composition is administered for at least 14 days.

42. The method of claim 41, wherein the pharmaceutical composition is administered for about 14 days, for about 30 days, for about 60 days, for about 84 days, for about 90 days, or continuously.

43. The method of any one of claims 1-42, wherein the pharmaceutical composition is administered as a single dose on each day the pharmaceutical composition is administered.

44. The method of any one of claims 1-42, wherein the pharmaceutical composition is administered in the form of several divided doses on each day the pharmaceutical composition is administered.

45. The method of any one of claims 1-44, the method further comprising administering one or more adjunct therapies.

46. The method of claim 45, wherein the method comprises administering one or more immune suppressive agents.

47. The method of claim 45, wherein the one or more immune suppressive agents are administered simultaneously with the pharmaceutical composition.

48. The method of claim 45, wherein the one or more immune suppressive agents are administered sequentially with the pharmaceutical composition.

49. The method of any one of claims 46-48, wherein the one or more immune suppressive agents are selected from the group consisting of azathioprine, calcineurin inhibitors, corticosteroids, cyclophosphamide, leflunomide, methotrexate, mycophenolate, and combinations thereof.

50. A method of assessing the efficacy of treating an inflammatory disorder with the composition described in any one of claims 1-24, the method comprising:
(a) obtaining a sample from a subject that has received treatment with the composition;
(b) measuring the level of one or more inflammatory cytokines in the sample; and
(c) if the level of the one or more inflammatory cytokines in the sample is lower than a control level for the one or more inflammatory cytokines, determining that the subject is responsive to the treatment.

51. The method of claim 50, wherein the one or more inflammatory cytokines are selected from IL-1β, IL-6, IL-10, IL-12p70, IL-17, IL-21, IL-23, IFN-α, IFN-γ, TNF-α, and CXCL13.

52. The method of claim 50 or 51, wherein the control level for the one or more inflammatory cytokines is the level for the one or more inflammatory cytokines obtained from the same subject before the start of treatment.

53. The method of any one of claims 50-52, wherein the inflammatory disorder is an autoimmune disorder.

54. The method of claim 53, wherein the autoimmune disorder is limbic encephalitis, rheumatoid arthritis (RA), systemic lupus erythematosus (SLE), Psoriasis, Crohn's disease or graft-versus-host disease (GVHD).

55. The method of any one of claims 50-52, wherein the inflammatory disorder is a neuroinflammatory disorder.

56. The method of claim 55, wherein the neuroinflammatory disorder is Alzheimer's disease (AD), Parkinson's disease (PD), stroke, seizure disorders, epilepsy, infantile spasms, or amyotrophic lateral sclerosis (ALS).

57. The method of any one of claims 50-56, wherein the subject is a mammal.

58. The method of claim 57, wherein the subject is a human.
